(19) United States
(12) Patent Application Publication
(10) Pub. No.: US 2011/0217248 A1
(43) Pub. Date: Sep. 8, 2011

(54) Topical Composition

(75) Inventors: Monique Spann-Wade, Chesterfield, MO (US); Kenton N. Fedde, St. Louis, MO (US)

(73) Assignee: ISW Group, Inc., St. Charles, MO (US)

(21) Appl. No.: 12/944,014

(22) Filed: Nov. 11, 2010

Related U.S. Application Data
(63) Continuation of application No. 11/842,201, filed on Aug. 21, 2007, now abandoned.
(60) Provisional application No. 60/824,642, filed on Sep. 6, 2006.
(60) Provisional application No. 60/893,888, filed on Mar. 9, 2007.

(51) Int. Cl. A61K 31/192; A61P 29/00; A61P 17/08; A61P 17/00; A61P 17/02; A61P 31/04; A61K 31/56; A61Q 17/04; A61K 31/196; A61K 31/167; A61K 31/44; A61K 31/16; A61K 31/40

(52) U.S. Cl. 424/59; 514/171; 514/570; 514/567; 514/629; 514/569; 514/352; 514/575; 514/420

(57) ABSTRACT

Topical compositions are disclosed that are useful for delivering a therapeutic level of an NSAID to a target within a subject having a local inflammatory disorder. A composition of the present invention comprises a Drug and a solvent system, wherein the solvent system comprises at least two solvent alcohols and wherein the solvent system is present in an amount sufficient to solubilize the Drug, the solvent system is a low alkanol system, and the composition is a single phase composition. Examplary solvent systems are those for which one of the at least two solvent alcohols is polyethylene glycol, glycerin, butylene glycol, diproylene glycol, propylene glycol, ethanol, isopropanol, or a derivative therof. Optionally the local inflammatory disorder is pseudofolliculitis barbae, dermatitis, psoriasis, wounds, or sunburn.

### PRIORITY

This application claims priority to U.S. Provisional Application Ser. No. 60/824,642 filed 6 Sep. 2006 and U.S. Provisional Application Ser. No. 60/893,888 (ISW P-0307) filed 9 Mar. 2007, both of which are hereby incorporated by reference herein in their entirety,

### TECHNICAL FIELD

The present invention relates to topical compositions, particularly topical compositions, which are used for applying pharmaceutical agents to the skin. The invention also relates to compositions for treating inflammation and for pain resulting from local stimulation of nociceptors in skin, bones, joints, and muscles and in skin disorders wherein inflammation is a component of the pathogenesis. An example of such an inflammatory skin disorder that relates to the present invention is pseudofolliculitis barbae.

### FIELD OF THE INVENTION

The pathogenesis of a wide variety of local disorders (e.g. skin, joints, muscle, and ligaments) involves an inflammatory process. Often, such disorders involve inflammatory cells (e.g., polymorphonuclear neutrophils and lymphocytes) infiltrating the skin with no overt or known infectious etiology. Symptoms of inflammatory skin conditions generally include erythema (redness), edema (swelling), pain, pruritus, increased surface temperature and loss of function.

While a range of treatments have been developed for local inflammatory conditions, none are completely effective or free of adverse side effects. Treatments for different inflammatory skin conditions typically include topical or oral steroids (e.g., for various types of eczema, acne, and erythema multiforme); ultraviolet light (e.g., for nummular eczema and mycosis fungoides); antibiotics, and other anti-inflammatory therapies.

In the past, corticosteroids have had the greatest importance for the treatment of inflammatory skin disorders. Weak to medium-strong corticosteroids (e.g. non-fluorinated derivatives of hydrocortisone) are mainly employed for the therapy of inflammatory, allergic and pruritic skin disorders. While short term treatment (a few days or weeks) with oral steroids is relatively safe, long term treatment (more than 3 months) may cause undesirable side effects including Cushing's syndrome, skin thinning, and increased susceptibility to infection.

There are also a variety of agents commonly used in medical practice which are non-narcotic and non-steroidal, but which nevertheless can be used to combat both inflammation and pain. These are the salicylates and also agents which are often termed by others as non-steroidal antiinflammatory drugs (NSAIDs).

There are now a variety of newer drugs available. Although the chemical structures of these newer agents vary quite widely, a common structural feature of many of these compounds is the presence of a carboxylic acid group (COON). For example, one group of NSAIDs consists of propionic acid derivatives (the so-called “profens,”e.g., ibuprofen), and another group of NSAIDs consists of acetic acid derivatives (e.g., indomethacin).

NSAIDs can cause gastric ulcers and bleeding on long term oral use. A goal of topical administration of NSAIDs is to deliver therapeutically effective levels of drug to the local target (e.g. nociceptors and inflammatory cells in the skin) while bypassing the stomach and preventing systemic delivery and associated side effects or adverse events,

Unfortunately, NSAIDs are often not well-absorbed when administered topically. Those topical formulations that do provide some absorption through the skin can result in substantial systemic delivery and often fail to provide therapeutic levels in the skin.

In addition, acute inflammation and pain are often treated by the topical administration of a counterirritant. In this regard, a widely-used agent is methyl salicylate, which is often applied to the skin in the form of an ointment or cream and which elicits a soothing, mildly-analgesic effect. However, methyl salicylate suffers from the disadvantage that it possesses an odor, which under certain circumstances, and to certain individuals, can be regarded as unpleasant.

U.S. Pat. No. 4,185,100 entitled “Topical Anti-Inflammatory Drug Therapy” generally describes topical treatment of an inflammatory condition of the skin comprising a non-steroidal anti-inflammatory agent and concurrently a topically active anti-inflammatory corticosteroid, These agents are applied in a dermatologically-acceptable, topical vehicle selected from the group consisting of creams, gels, ointments, powders, aerosols and solutions suitable for topical administration.

Kyuki et al., “Anti-Inflammatory Effect of Diclofenac-Sodium Ointment (Cream) in Topical Application”, Japan J. Pharmacol. 33, 121-132 (1983) describes the anti-inflammatory effect of a diclofenac-sodium. Ointments were prepared with three kinds of bases: lithophilic, emulsion (cream) and gel bases and their anti-inflammatory effects were compared. The cream base was reported by Kyaki et al. to have the most potent effect.

European Patent Application 0151953 entitled “Topical Drug Release System” describes on page 10-11 an ibuprofen CARBOPOL gel system containing ibuprofen, propylene glycol, water, CARBOPOL 940 (polyacrylic acid polymer) and di-isopropanolamine, as an illustrative example of a pharmaceutical composition for percutaneous absorption by topical application made in two liquid drug-containing phases, which are to be mixed together in situ just before use to form a supersaturated drug-containing gel. The EPO application discloses a non-alcoholic gel system for delivering ibuprofen topically.

US Patent No. 20060067958 teaches that “alcohol, particularly ethanol, is generally known as a permeation enhancer for topical drugs” and that increased rate of drug absorption leads to faster onset of action and enhanced efficacy. The applicants describe a need for an alcoholic gel containing with very low levels of water, preferably less than 20% w/w, for various reasons, e.g. because, the presence of high level of water in the composition can retard the absorption rate. Moreover, they teach that the drug may not be soluble in the presence of water such as for example when the drug preferentially forms an insoluble hydrate.

U.S. Pat. No. 5,093,133 entitled “Method for percutaneous delivery of ibuprofen using hydroalcoholic gel” describes a hydroalcoholic gel comprising ibuprofen, a hydroxypropylcellulose or polyacrylic acid polymer. Such hydroalcoholic gels are purported to be significantly more effective than a cream, non-alcoholic or hydroalcoholic gel of pH above 7.0 for purposes of percutaneous delivery of ibuprofen through the skin. The patent also describes that certain non-volatile solvents such as. propylene glycol improves the spreading properties and aestethics of the gel. The patent teaches that propylene glycol is not critical in the sense that it does not appear to alter the delivery rate of ibuprofen through the skin. The patent further describes using the enatiomer of ibuprofen and adding alkalinizing agent to the formulation to increase percutaneous absorption of the drug.

U.S. Pat. No. 4,533,546 entitled “Anti-Inflammatory Analgesic Gelled Ointments” to Kishi et al. discloses NSAID (e.g. ibuprofen) containing hydroalcoholic gels having a pH in the range of 7.0 to 9.0. The gel ointment comprises a phenylacetic acid anti inflammatory compound, a carboxyvinyl polymer, a water-soluble organic amine (e.g. triethanolamine), and water wherein the amount of organic amine is such that the gel ointment has a pH in the range of 7.0 to 9.0 and preferably 7.3 to 7.8.

Seth, in “Percutaneous absorption of Ibuprofen from Different formulations” (Drug Res 43: 919-921, 1993) showed that absorption in humans (assessed by measuring plasma levels), is highest in hydroalcoholic gels when compared to polyethylene glycol based compositions.

Treffel et al. in “Ibuprofen epidermal levels after topical applications in vitro:” (British J of Derm 129:286-291, 1993) show rapid and high penetration of ibuprofen through the skin from hydroalcoholic gels but that absorption does not follow Fick's law. Instead, a 10% ibuprofen had lower drug absorption than a 5% gel. Moreover, they reason that when the solubility limit of the alcohol is exceeded, the drug precipitates and goes into a suspension, and remains as a solid film on the skin surface. Hence, Treffel et al teach high alcohol compositions with less than 10% ibuprofen.

U.S. Pat. No. 5,976,566 state that “Surprisingly, it has been found that when propylene glycol is used in the vehicle for the ibuprofen formulations, but not for other NSAIDS, such as diclofenac, ketoprofen, piroxicam, the initial flux rate of ibuprofen decreased as the amount of propylene glycol (PG) increased.”

Topical gels containing ibuprofen have been described in U.S. Pat. No. 6,277,362 entitled “After shave treatment preparation” to Ita, issued Aug. 21, 2001, for treatment of pseudofolliculitis barbae (PFB). Pseudofolliculitis barbae is a skin disorder primarily affecting subjects who shave curly hairs. A coiled hair tends to grow by curving backward toward the skin. Over the course of a single day's growth, the tip of the hair shaft may press back into the skin. Since the razor leaves a sharp sheared edge on the hair tip, the hair may actually penetrate the skin and continue proceeding inward.

The epidermis (i.e. the outermost layer of the skin) contains keratinocytes. In response to penetration (e.g. by a hair), keratinocytes and other nonhematopoietically-derived resident cells produce various cytokines which stimulate migration of T cells and expression of adhesion molecules. As a result, inflammatory cells (e.g., polymorphonuclear neutrophils and lymphocytes) infiltrate the skin (from the dermis) resulting in a swollen bump in the region.

Full blown PFB is typically characterized by irritating bumps, itchiness, and discoloration of the affected areas. PFB becomes part of an accelerating cycle. The bumps are present the next time shaving takes place, resulting in a cut of the raised area and further irritation. Additionally; complications of PFB include cellulitis, furunculosis, hyperpigmentation, bacterial superinfection, and hypertrophic or keloid scars. Secondary bacterial infection can also result from PFB.

Prior art known to the inventors concerning the subject of PFB includes the following references:

U.S. Pat. No. 3,981,681, issued to Mario de la Guarida, on Sep. 21, 1976.

U.S. Pat. No. 4,228,163, issued to William E. Bliss, on Oct. 14, 1980;

U.S. Pat. No. 4,525,344, issued to Ronald J. Tutsky, on Jun. 25, 1985;

U.S. Pat. No. 4,775,530, issued to Nicholas V. Perricone, on Oct. 4, 1988; and

U.S. Pat. No. 5,034,221, issued to Steven E. Rosen et al., on Jul. 23, 1991.

Typically, topical formulations, and particularly gel formulations, are thickened using well-known polymeric thickeners, such as the CARBOPOL® materials which are copolymers or polymers of polyacrylic acids.

What is needed in the art is a topical composition that delivers an effective concentration of an active drug to treat an inflammatory skin condition with with desireable therapeutic, pharmacokinetic, pharmacodymanimic, and safety profiles (e.g. low systemic delivery).

### SUMMARY OF THE INVENTION

New compositions have been discovered that when topically applied, deliver therapeutic levels of an agent with anti-inflammatory activity (the “Drug”) to the local targets in an individual with a local inflammatory disorder.

Surprisingly, it has been discovered that compositions of the present invention have one or more advantageous pharmacodynamic, pharmacokinetic, and/or therapeutic properties and provide therapeutic levels of NSAID for a diverse range of local inflammatory disorders. Moreover, therapeutic levels of an NSAID are attained with minimal systemic delivery using low alkanol compositions, that is compositions containing less alkanol than about any of the following: 65% or 45% or 25% or 10%.

The present invention provides therapeutically effective compositions comprising a Drug and a solvent system, wherein:

the solvent system comprises at least two solvent alcohols;

the solvent system is present in an amount sufficient to solubilize the Drug;

the Drug is in an amount of at least 5% by weight;

and the composition is a single phase composition.

By way of example, the Drug is an NSAID with a high solubility in polyethylene glycol, propylene glycol, or alkanols. Non-limiting examples of an NSAID with a high solubility include ketoprofen, ibuprofen, naproxen, flurbiprofen, diclofenac, and acetaminophen.

High solubility means, by way of example, greater than 5% Drug at saturation.

Optionaly, the Drug is an NSAID prodrug of the phenylacetic acid-type NSAID.

Optionally, the composition further comprises a prodrug and a Drug other than a prodrug.

Optionally, a present composition further comprises one or more of an antibiotic, an antifungal, a steroid, an antipsoriatic agent, clindamycin, cyclosporine, a UVA and UVB blocker, or a botanical agent.

Optionally, a present composition further comprises at least one excipient selected from water, thickeners, humectants, keratolytics, oils, emollients, surfactants, preservatives, colorants, UV blockers, antioxidants, and perfumes.

Also provided is a method of treating a local inflammatory disorder comprising applying to the skin of a subject in need thereof a composition of the present invention wherein such application results in local delivery of therapeutic levels of the Drug without substantial delivery into the subject's circulation.

Optionally the inflammatory skin disorder is pseudofolliculitis (e.g. barbae type), dermatitis, psoriasis, wounds, tinea, dermatophytoses, nonimmunologic urticaria, herpes infections (e.g. zoster or simplex type), or sunburn.

### BRIEF DESCRIPTION OF THE DRAWINGS

FIG. 1 shows the UV chromatogram (220 nm) of HPLC following injection of composition stored 3 months ˜25° C.

FIG. 2 shows the positive ESI mass spectrum for the Ibuprofen peak.

FIG. 3 shows the UV spectrum for the Ibuprofen.

FIG. 4 shows the positive ESI mass spectrum obtained from the prodrug.

FIG. 5 shows the UV spectrum obtained from the prodrug

FIG. 6 shows the effect of two different pHs on prodrug formation.

FIG. 7 shows the effect of pH and Drug concentration on prodrug formation.

FIG. 8 shows the relationship between water and Naproxen concentrations at saturation, where Panel A shows linear regression for each composition and Panel B shows the linear regression for all of the data combined.

FIG. 9 shows the relationship between water and Ketoprofen concentrations at saturation.

FIG. 10 shows the relationship between water and Ibuprofen concentrations at saturation.

FIG. 11 shows the relationship between water and Acetaminophen concentrations at saturation, where Panel A shows the linear regression for all of the data combined and Panel B shows linear regression for each composition individualy.

### DETAILED DESCRIPTION OF THE INVENTION

### Definitions

As used herein, the following definitions apply:

“Alkanol” means dermatologically acceptable monohydric unsubstituted alkyl alcohols represented by the formula R—OH, wherein R represents an alkyl radical. Non limiting examples of alkanols include ethanol, isopropanol, and benzyl alcohol.

“Disorder” means any abnormal pathology. A disorder can be inherited, infectious, acquired, induced (e.g. contact dermatitis or inflammation following surgical incision), chronic, or acute.

“Drug” means one or more dermatologically acceptable agents with anti-inflammatory activity which includes agents that blunt an inflammatory reaction, irrespective to the underlying mechanism (e.g. inhibition of prostaglandin synthesis, leukotriene production, macrophage function, etc). “Drug” includes small molecules with elucidated structures (e.g. a nonsteroidal inflammatory drug or NSAID). “Drug” also includes biological or botanical extracts or preparations (a “botanical agent”). Drug and NSAID include polymorphs, crystal habits thereof, prodrugs and isomers thereof (including optical, geometric and tautomeric isomers), enantiomers, salts, solvates and complexes thereof and solvates and complexes of salts thereof.

“Excipients” means any material that is combined with a drug in order to produce a drug dosage form. Such Excipients can be combined in order to produce a desired skin feel or to facilitate drug delivery. Non-limiting examples of excipients include, for example, water, thickeners, humectants, keratolytics, oils, emollients, surfactants, preservatives, colorants, UV blockers (e.g. UVA and UVB), antioxidants, perfumes, mineral oil, liquid petrolatum, and white petrolatum. An excipient may also serve a solvent function. For example, polysorbate and panthenol have properties as a humectant and as a solvent.

“Local Dose” means amount or concentration of drug that reaches a local target.

“Local Inflammatory disorder” means a disorder wherein an inflammatory process is a component of a disorder of a local target. Examples of local inflammatory disorders occur through this application but generally include any of the conditions of pain, swelling, edema, redness, tissue damage, assault to skin, cellular injury, etc. Such disorders generally are treatable by cox-1 inhibitors, cox-2 inhibitors, or steroids.

“Local Targets” means, tissue affected by a disorder that can be treated by delivery of a Drug by present compositions—by way of example, skin, joints, muscle, and ligaments.

“%”, in reference to a concentration of a component of a composition, means the ratio of weight of a component to total weight expressed as a percent, unless otherwise stated.

“Prevent”, “preventing”, or “prevention” means any reduction, no matter how slight, of a subject's predisposition or risk for developing pain, inflammation, an inflammation-related disorder, and/or a disorder with an inflammation related aspect. For purposes of prevention, the subject is any subject, and preferably is a subject that is at risk for, or is predisposed to, developing a local inflammatory disorder. The term “prevention” includes either preventing the onset of clinically evident inflammation altogether or preventing the onset of preclinically evident inflammation in individuals at risk. Also intended to be encompassed by this definition is the prevention of initiation for inflammatory cells or to arrest or reverse the progression of the inflammation cascade. This includes prophylactic treatment of those at risk of developing the inflammation.

“Present”, in the context of, by way of example, “present drug” or “present composition” refers to the invention first disclosed hereon (e.g. drug of the present invention or composition of the present invention).

“Prodrug” means a pharmacologically inactive or less active chemical derivative of an NSAID that can be converted to a more active form (“parent drug”) by an enzymatic or chemical hydrolysis in vivo. The prodrug consists of the parent drug covalently linked to another compound (the “pro-moiety”). Optionally, prodrug does not include an NSAID derivative formed by esterification at an NSAID carboxylic acid functionality with an acyloxyalkyl radical. “Prodrug ester” denotes a prodrug wherein the pro-moiety is in ester linkage to the parent drug.

“Safe and effective amount” means an amount of the composition which is sufficient to provide a level of treatment to a condition, but is not so great as to provide side effects to the user that are so great as to make treatment medically imprudent.

“Single phase composition” means that the Drug is predominantly or completely dissolved in the solvent system and the solvents that constitute the solvent system are predominantly or completely miscible together. Single phase composition is meant to distinguish present compositions from emulsions, colloidal mixtures, 2 phase compositions (e.g. oil and water), compositions where an appreciable amount (by way of example, about 5%) of the composition is insoluble, and the like. A present composition can be a single phase composition despite the mere presence of an insoluble excipient such as a thickening agent or despite a phase separation upon long term storage.

“Solubilize” as it pertains to the solvent system and the Drug, means that the solvent system makes the drug soluble in the system. Optionally, solubilize can additionally mean that the Drug is dissolved in the solvent system.

“Subject” or “individual” as it pertains to one infected with a local skin disorder means a human or a non-human mammal.

“Systemic delivery”, as it pertains to a topically applied Drug, means delivery of the Drug into the vascular bed and entry into circulation (i.e. blood). Accordingly, systemic delivery can be quantified by measuring the resultant levels of the Drug in plasma, serum, or whole blood. “Levels” can peak achieved levels ([Cₘₐₓ]) or an integrated level (i.e. area under the curve [AUC]).

“Therapeutically effective” or “treatment” in context of a formulation means that when applied to the skin according to sound medical practice, it causes a demonstratable effect to diminish or prevent a local inflammatory response. Such demonstration can be at the gross pathological level (e.g. visual reduction of swelling, redness, or any characteristic skin pathology; e.g. skin bump in PFB), subjective level (subject's perception of pain), or through biochemical analysis of surrogate or direct markers of inflammation or inflammatory disease. Therapeutically effective or Treatment can be curative, palliative and/or prophylactic or preventive treatment. It is not meant to indicate a quantitative effect, but rather that there has been a clinically observable beneficial effect. For example, prophylactic treatment includes a situation where a composition of the present invention is administered to a subject before symptoms are observable and symptoms do not subsequently occur or occur to a lesser degree than without administration.

PFB is a good means for assessing therapeutic efficacy because there is often no infectious agent. Accordingly, regressing or preventing the formation of inflamed hair follicles are demonstrations of therapeutic efficacy.

It should readily be recognized by one skilled in the art that compositions with therapeutic efficacy demonstrated against PFB also will have utility against other local inflammatory disorders.

“Therapeutic level” (or “therapeutically effective levels”) means a local concentration of Drug that results in therapeutic efficacy. The weight of Drug per unit tissue per interval of time necessary to result in therapeutic efficacy is dependant upon the inflammatory disorder, the severity thereof, and the subject.

“Thickening agent” means any agent useful as an aid to thicken or add structure to a topical formulations. These agents impart physical stability and increased viscosity. Non-limiting examples of thickening agents are gums and natural polysaccharides, mineral thickeners, oils, and synthetic polymeric thickeners. Additionally, a thickening agent refers to one or more agents that, in combination, result in a viscosity suitable for dermatologic applications.

“Topically acceptable” and “dermatologically acceptable” composition means that when applied to the skin, there is no substantial skin irritation under circumstances of normal usage with typical patients.

“Topically active” means an agent that, when applied to the skin in a dermatologic composition, can deliver an activity that has therapeutic efficacy against a local target.

“Viscosity” means liquid fluidity as measured by a Brookfield DV-III Ultra Programmable Rheometer, spindle #LV4, 10 rpm or equivalents (e.g. Brookfield Model R/S PLUS-CPSP1Cone/Plate Rheometer).

### Compositions

The present invention provides therapeutically effective compositions comprising a Drug and a solvent system, wherein the solvent system comprises at least two solvent alcohols and wherein the solvent system is present in an amount sufficient to solubilize the Drug, wherein the Drug is in an amount of at least 5% by weight, wherein one of the at least two solvent alcohols is a polyethylene glycol, a propylene glycol, glycerin, a polyether polyol, butylene glycol; an alkene glycol, or a glycerol derivative and wherein the composition is a single phase composition.

It has surprisingly been discovered that present compositions, when applied regularly to the skin (e.g. twice per day or less frequently), deliver therapeutic levels of Drug to local targets. Optionally, such levels can be achieved in low alkanol compositions, that is compositions containing less alkanol than about any of the following: 65% or 45% or 25% or 10%.

Delivery of therapeutic levels of Drug is due, in part, to the high concentration of Drug in the compositions, for example, more than about: 10% or 15% or 20%, or more. It has been discovered that high concentrations of Drug can be solubilized in present compositions having a solvent system comprising at least two solvent alcohols. Examples of solvent systems are those for which at least two solvent alcohols are selected are polyethylene glycol, glycerin, butylene glycol, diproylene glycol, propylene glycol, ethanol, and isopropanol.

Superior Drug Solubility in Present Compositions

The high concentration of Drug obtainable in present compositions is due to, in part, the surprising discovery that present solvent systems can solubilize more (e.g. about 20% more or about 75% or more) than the sum of the amounts predicted by the solubilities of the Drug in the individual solvent alcohols (the “super solvent effect”; e.g. a 20% or 75% super solvent effect).

Low alkanol compositions of the present invention show desirable pharmacokinetic, pharmacodynamic, and therapeutic profiles (e.g. when compared to hydroalcoholic gels). This is surprising in view of U.S. Pat. No. 5,093,133 that teaches superiority of hydroalcoholic gels.

Desirable pharmacokinetics of Drug in present compositions with a solvent system comprising propylene glycol is unexpected in view of U.S. Pat. No. 5,093,133 that states that propylene glycol does not appear to alter the delivery rate of ibuprofen through the skin.

Present compositions with a solvent system comprising propylene glycol show a present flux rate that is desirable (e.g. when compared to hydroalcoholic gels)—surprising in view of the teaching of U.S. Pat. No. 5,976,566

Low alkanol compositions of the present invention with a solvent system comprising polyethylene glycol have desirable pharmacokinetic and pharmacodynamic properties (e.g. when compared to hydroalcoholic gels)—surprising in view of Seth (Drug Res 43: 919-921, 1993) that showed absorption in humans (assessed by measuring plasma levels), is highest in hydroalcoholic gels when compared to polyethylene glycol based compositions.

This surprising effect of the solvent system on NSAID solubility has some especially beneficial consequences. In addition to providing for higher Drug concentrations, present compositions are especially stable with respect to Drug precipitation under adverse storage conditions, for example upon long term storage, low humidity, or at cold temperatures.

It has also been surprisingly discovered that solvent systems of the present invention have emollient affects at concentrations taught herein. For example, instant formulations have propertied beneficial to conditions where erythema and/or pain are components of the pathology. For example, instant formulations substantially reduce paid and redness associated with atopic dermatitis including in cases relatively refractory to other accepted treatments.

In one embodiment, the solvent system comprises at least two of polyethylene glycol (for example, a “PEG” optionally having a molecular weight less than about 1100), propylene glycol, ethanol, or isopropanol. In compositions where the NSAID is ibuprofen, the NSAID is present in either an amount of about 25% or of about 50% to either about 150% or to about 175% of the amount represented by the Formula 1.

0.25 [PG %]+0.33 [PEG %]+[EtOH %]+0.91 [IPA%]=[NSAID %] Formula 1

Optionally, the NSAID is present in an amount of about 100% to about 200% or more of the amount according to Formula 1.

Formula 1 is derived from Table 25. Accordingly, formulae for other Drugs can also be derived from Table 25

Useful ranges of optional compositions of the present invention are set forth in Table 1 (values are in % by weight). Each of these useful ranges demonstrate superior solvent properties as exemplified in the Tables and examples herein. “NSAID I, II, and III represent examples of three different ranges of Drug useful in the corresponding compositions. One skilled in the art should readily notice that in some of the formulations, the amount of Drug is not limited so much by the solubility of the Drug in the solvent system but by the total amount of the composition components (i.e., sum of the components must equal 100%). In Table 1, the asterisk indicates an upper limit bound by either the Drug solubility or by weight (i.e. to yield 100%).

With the teaching contained herein, useful compositions of the present invention can be defined mathematically. For example, solubilities in solvent alcohols of NSAIDs useful herein are set forth in Table 25 and the super-solvent effects are set forth in Table 26 through Table 30. Using such information for ibuprofen, for example, the following formulae can be developed:

useful for propylene glycol/PEG solvent systems, is (0.25 [PG %]+0.33 [PEG %])1.75≧[NSAID %]. Formula 2,

useful for propylene glycol/alkanol solvent systems, is (0.25 [PG %]+[EtOH %]+0.91 [IPA %])1.50≧[NSAID %]. Formula 3,

useful for PEG/alkanol solvent systems, is (0.33 [PEG %] +[EtOH %]+0.91 [IPA %]1.25≧[NSAID %]. Formula 4,

useful for propylene glycol/PEG/ethanol solvent systems, is (0.25 [PG %]+0.33 [PEG %]+[EtOH %])1.5≧[NSAID %]. Formula 5,

useful for propylene glycol/PEG/isopropanol solvent systems, is (0.25 [PG %]+0.33 [PEG %]+[EtOH %])1.67≧[NSAID %] Formula 6,

useful for propylene glycol/PEG/isopropanol solvent systems, is (0.33 [PEG %]+1 [EtOH %]+0.91 [IPA %])1.33≧[NSAID %]. Formula 7,

useful for propylene glycol/PEG/isopropanol solvent systems, is. (0.25 [PG %]+[EtOH %]+0.91 [IPA %]) 0.5≧[NSAID %] Formula 8,

useful for propylene glycol/PEG/isopropanol solvent systems, is (0.33 [PEG %]+0.25 [PG %]+[EtOH %]+0.91 [IPA %])1.62≧[NSAID %]. Formula 9,

Similarly, data presented herein, combined with the teaching herein, provided a mean to determine formulae for ketoprofen, naproxen, flurbiprofen, acetaminophen, diclofenac, and other NSAIDs of the present invention. Generally, high Drug compositions are made by adding only sufficient amount of the solvent system to make a saturated, near saturated, or at least about one-fourth saturated solution.

TABLE 1
Useful Component Ranges of Present Compositions

| | | | NSAID | NSAID | NSAID |
|---|---|---|---|---|---|
| PG | PEG | Alkanol | I | II | III |
| 0-5 | 0-5 | 0-5 | 6-10 | 10-10 | |
| 0-5 | 0-5 | 5-10 | 6-10 | 10-15 | 15-15 |
| 0-5 | 0-5 | 7-15 | 6-10 | 10-15 | 15-20 |
| 0-5 | 0-5 | 10-20 | 6-10 | 10-15 | 15-25 |
| 0-5 | 0-5 | 15-30 | 6-10 | 10-15 | 15-35 |
| 0-5 | 0-5 | 20-40 | 6-10 | 10-15 | 15-45 |
| 0-5 | 0-5 | 30-50 | 6-10 | 10-15 | 15-* |
| 0-5 | 0-5 | 40-60 | 6-10 | 10-15 | 15-* |
| 0-5 | 5-10 | 0-5 | 6-10 | 10-13 | |
| 0-5 | 5-10 | 5-10 | 6-10 | 10-15 | 15-18 |
| 0-5 | 5-10 | 7-15 | 6-10 | 10-15 | 15-23 |
| 0-5 | 5-10 | 10-20 | 6-10 | 10-15 | 15-28 |
| 0-5 | 5-10 | 15-30 | 6-10 | 10-15 | 15-38 |
| 0-5 | 5-10 | 20-40 | 6-10 | 10-15 | 15-30 |
| 0-5 | 5-10 | 30-50 | 6-10 | 10-15 | 15-30 |
| 0-5 | 5-10 | 40-60 | 6-10 | 10-15 | 15-* |
| 0-5 | 7-15 | 0-5 | 6-10 | 10-15 | 15-* |
| 0-5 | 7-15 | 5-10 | 6-10 | 10-15 | 15-21 |
| 0-5 | 7-15 | 7-15 | 6-10 | 10-15 | 15-26 |
| 0-5 | 7-15 | 10-20 | 6-10 | 10-15 | 15-31 |
| 0-5 | 7-15 | 15-30 | 6-10 | 10-15 | 15-25 |
| 0-5 | 7-15 | 20-40 | 6-10 | 10-15 | 15-51 |
| 0-5 | 7-15 | 30-50 | 6-10 | 10-15 | 15-* |
| 0-5 | 7-15 | 40-60 | 6-10 | 10-15 | 15-* |
| 0-5 | 10-20 | 0-5 | 6-10 | 10-15 | 15-* |
| 0-5 | 10-20 | 5-10 | 6-10 | 10-15 | 15-24 |
| 0-5 | 10-20 | 7-15 | 6-10 | 10-15 | 15-29 |
| 0-5 | 10-20 | 10-20 | 6-10 | 10-15 | 15-34 |
| 0-5 | 10-20 | 15-30 | 6-10 | 10-15 | 15-44 |
| 0-5 | 10-20 | 20-40 | 6-10 | 10-15 | 15-* |
| 0-5 | 10-20 | 30-50 | 6-10 | 10-15 | 15-* |
| 0-5 | 10-20 | 40-60 | 6-10 | 10-15 | 15-* |
| 0-5 | 15-30 | 0-5 | 6-10 | 10-15 | 15-* |
| 0-5 | 15-30 | 5-10 | 6-10 | 10-15 | 15-30 |
| 0-5 | 15-30 | 7-15 | 6-10 | 10-15 | 15-35 |
| 0-5 | 15-30 | 10-20 | 6-10 | 10-15 | 15-40 |
| 0-5 | 15-30 | 15-30 | 6-10 | 10-15 | 15-50 |
| 0-5 | 15-30 | 20-40 | 6-10 | 10-15 | 15-* |
| 0-5 | 15-30 | 30-50 | 6-10 | 10-15 | 15-* |
| 0-5 | 15-30 | 40-60 | 6-10 | 10-15 | 15-* |
| 0-5 | 20-40 | 0-5 | 6-10 | 10-15 | 15-* |
| 0-5 | 20-40 | 5-10 | 6-10 | 10-15 | 15-36 |
| 0-5 | 20-40 | 7-15 | 6-10 | 10-15 | 15-41 |
| 0-5 | 20-40 | 10-20 | 6-10 | 10-15 | 15-* |
| 0-5 | 20-40 | 15-30 | 6-10 | 10-15 | 15-* |
| 0-5 | 20-40 | 20-40 | 6-10 | 10-15 | 15-* |
| 0-5 | 20-40 | 30-50 | 6-10 | 10-15 | 15-* |
| 0-5 | 20-40 | 40-60 | 6-10 | 10-15 | 15-* |
| 0-5 | 30-50 | 0-5 | 6-10 | 10-15 | 15-* |
| 0-5 | 30-50 | 5-10 | 6-10 | 10-15 | 15-41 |
| 0-5 | 30-50 | 7-15 | 6-10 | 10-15 | 15-* |
| 0-5 | 30-50 | 10-20 | 6-10 | 10-15 | 15-* |
| 0-5 | 30-50 | 15-30 | 6-10 | 10-15 | 15-* |
| 0-5 | 30-50 | 20-40 | 6-10 | 10-15 | 15-* |
| 0-5 | 30-50 | 30-50 | 6-10 | 10-15 | 15-* |
| 0-5 | 30-50 | 40-60 | 6-10 | 10-15 | 15-* |
| 0-5 | 40-60 | 0-5 | 6-10 | 10-15 | 15-42 |
| 0-5 | 40-60 | 5-10 | 6-10 | 10-15 | 15-* |
| 0-5 | 40-60 | 7-15 | 6-10 | 10-15 | 15-* |
| 0-5 | 40-60 | 10-20 | 6-10 | 10-15 | 15-* |
| 0-5 | 40-60 | 15-30 | 6-10 | 10-15 | 15-* |
| 0-5 | 40-60 | 20-40 | 6-10 | 10-15 | 15-* |
| 0-5 | 40-60 | 30-50 | 6-10 | 10-15 | 15-* |
| 0-5 | 40-60 | 40-60 | 6-10 | 10-15 | 15-20 |
| 5-10 | 0-5 | 0-5 | 6-10 | 10-12 | |
| 5-10 | 0-5 | 5-10 | 6-10 | 10-15 | 15-17 |
| 5-10 | 0-5 | 7-15 | 6-10 | 10-15 | 15-22 |
| 5-10 | 0-5 | 10-20 | 6-10 | 10-15 | 15-27 |
| 5-10 | 0-5 | 15-30 | 6-10 | 10-15 | 15-37 |
| 5-10 | 0-5 | 20-40 | 6-10 | 10-15 | 15-47 |
| 5-10 | 0-5 | 30-50 | 6-10 | 10-15 | 15-* |
| 5-10 | 0-5 | 40-60 | 6-10 | 10-15 | 15-* |
| 5-10 | 5-10 | 0-5 | 6-10 | 10-15 | 15-* |
| 5-10 | 5-10 | 5-10 | 6-10 | 10-15 | 15-20 |
| 5-10 | 5-10 | 7-15 | 6-10 | 10-15 | 15-25 |
| 5-10 | 5-10 | 10-20 | 6-10 | 10-15 | 15-30 |
| 5-10 | 5-10 | 15-30 | 6-10 | 10-15 | 15-40 |
| 5-10 | 5-10 | 20-40 | 6-10 | 10-15 | 15-50 |
| 5-10 | 5-10 | 30-50 | 6-10 | 10-15 | 15-* |
| 5-10 | 5-10 | 40-60 | 6-10 | 10-15 | 15-* |
| 5-10 | 7-15 | 0-5 | 6-10 | 10-15 | 15-* |
| 5-10 | 7-15 | 5-10 | 6-10 | 10-15 | 15-23 |
| 5-10 | 7-15 | 7-15 | 6-10 | 10-15 | 15-28 |
| 5-10 | 7-15 | 10-20 | 6-10 | 10-15 | 15-33 |
| 5-10 | 7-15 | 15-30 | 6-10 | 10-15 | 15-43 |
| 5-10 | 7-15 | 20-40 | 6-10 | 10-15 | 15-* |
| 5-10 | 7-15 | 30-50 | 6-10 | 10-15 | 15-* |
| 5-10 | 7-15 | 40-60 | 6-10 | 10-15 | 15-* |
| 5-10 | 10-20 | 0-5 | 6-10 | 10-15 | 15-* |
| 5-10 | 10-20 | 5-10 | 6-10 | 10-15 | 15-26 |
| 5-10 | 10-20 | 7-15 | 6-10 | 10-15 | 15-31 |
| 5-10 | 10-20 | 10-20 | 6-10 | 10-15 | 15-36 |
| 5-10 | 10-20 | 15-30 | 6-10 | 10-15 | 15-46 |
| 5-10 | 10-20 | 20-40 | 6-10 | 10-15 | 15-* |
| 5-10 | 10-20 | 30-50 | 6-10 | 10-15 | 15-* |
| 5-10 | 10-20 | 40-60 | 6-10 | 10-15 | 15-* |
| 5-10 | 15-30 | 0-5 | 6-10 | 10-15 | 15-27 |
| 5-10 | 15-30 | 5-10 | 6-10 | 10-15 | 15-32 |
| 5-10 | 15-30 | 7-15 | 6-10 | 10-15 | 15-37 |
| 5-10 | 15-30 | 10-20 | 6-10 | 10-15 | 15-42 |
| 5-10 | 15-30 | 15-30 | 6-10 | 10-15 | 15-* |
| 5-10 | 15-30 | 20-40 | 6-10 | 10-15 | 15-* |
| 5-10 | 15-30 | 30-50 | 6-10 | 10-15 | 15-* |
| 5-10 | 15-30 | 40-60 | 6-10 | 10-15 | 15-* |
| 5-10 | 20-40 | 0-5 | 6-10 | 10-15 | 15-33 |
| 5-10 | 20-40 | 5-10 | 6-10 | 10-15 | 15-38 |
| 5-10 | 20-40 | 7-15 | 6-10 | 10-15 | 15-43 |
| 5-10 | 20-40 | 10-20 | 6-10 | 10-15 | 15-* |
| 5-10 | 20-40 | 15-30 | 6-10 | 10-15 | 15-* |
| 5-10 | 20-40 | 20-40 | 6-10 | 10-15 | 15-* |
| 5-10 | 20-40 | 30-50 | 6-10 | 10-15 | 15-* |
| 5-10 | 20-40 | 40-60 | 6-10 | 10-15 | 15-* |
| 5-10 | 30-50 | 0-5 | 6-10 | 10-15 | 15-39 |
| 5-10 | 30-50 | 5-10 | 6-10 | 10-15 | 15-44 |
| 5-10 | 30-50 | 7-15 | 6-10 | 10-15 | 15-* |
| 5-10 | 30-50 | 10-20 | 6-10 | 10-15 | 15-* |
| 5-10 | 30-50 | 15-30 | 6-10 | 10-15 | 15-* |
| 5-10 | 30-50 | 20-40 | 6-10 | 10-15 | 15-* |
| 5-10 | 30-50 | 30-50 | 6-10 | 10-15 | 15-* |
| 5-10 | 30-50 | 40-60 | 6-10 | 10-15 | 15-20 |
| 5-10 | 40-60 | 0-5 | 6-10 | 10-15 | 15-44 |
| 5-10 | 40-60 | 5-10 | 6-10 | 10-15 | 15-* |
| 5-10 | 40-60 | 7-15 | 6-10 | 10-15 | 15-* |
| 5-10 | 40-60 | 10-20 | 6-10 | 10-15 | 15-* |
| 5-10 | 40-60 | 15-30 | 6-10 | 10-15 | 15-35 |
| 5-10 | 40-60 | 20-40 | 6-10 | 10-15 | 15-30 |
| 5-10 | 40-60 | 30-50 | 6-10 | 10-15 | 15-20 |
| 5-10 | 40-60 | 40-60 | 6-10 | 10-15 | 15-10 |
| 7-15 | 0-5 | 0-5 | 6-10 | 10-14 | |
| 7-15 | 0-5 | 5-10 | 6-10 | 10-15 | 15-19 |
| 7-15 | 0-5 | 7-15 | 6-10 | 10-15 | 15-24 |
| 7-15 | 0-5 | 10-20 | 6-10 | 10-15 | 15-29 |
| 7-15 | 0-5 | 15-30 | 6-10 | 10-15 | 15-39 |
| 7-15 | 0-5 | 20-40 | 6-10 | 10-15 | 15-49 |
| 7-15 | 0-5 | 30-50 | 6-10 | 10-15 | 15-* |
| 7-15 | 0-5 | 40-60 | 6-10 | 10-15 | 15-* |
| 7-15 | 5-10 | 0-5 | 6-10 | 10-15 | 15-* |
| 7-15 | 5-10 | 5-10 | 6-10 | 10-15 | 15-22 |
| 7-15 | 5-10 | 7-15 | 6-10 | 10-15 | 15-27 |
| 7-15 | 5-10 | 10-20 | 6-10 | 10-15 | 15-32 |
| 7-15 | 5-10 | 15-30 | 6-10 | 10-15 | 15-42 |
| 7-15 | 5-10 | 20-40 | 6-10 | 10-15 | 15-* |
| 7-15 | 5-10 | 30-50 | 6-10 | 10-15 | 15-* |
| 7-15 | 5-10 | 40-60 | 6-10 | 10-15 | 15-* |
| 7-15 | 7-15 | 0-5 | 6-10 | 10-15 | 15-* |
| 7-15 | 7-15 | 5-10 | 6-10 | 10-15 | 15-25 |
| 7-15 | 7-15 | 7-15 | 6-10 | 10-15 | 15-30 |
| 7-15 | 7-15 | 10-20 | 6-10 | 10-15 | 15-35 |
| 7-15 | 7-15 | 15-30 | 6-10 | 10-15 | 15-45 |
| 7-15 | 7-15 | 20-40 | 6-10 | 10-15 | 15-* |
| 7-15 | 7-15 | 30-50 | 6-10 | 10-15 | 15-* |
| 7-15 | 7-15 | 40-60 | 6-10 | 10-15 | 15-* |
| 7-15 | 10-20 | 0-5 | 6-10 | 10-15 | 15-* |
| 7-15 | 10-20 | 5-10 | 6-10 | 10-15 | 15-28 |
| 7-15 | 10-20 | 7-15 | 6-10 | 10-15 | 15-33 |
| 7-15 | 10-20 | 10-20 | 6-10 | 10-15 | 15-38 |
| 7-15 | 10-20 | 15-30 | 6-10 | 10-15 | 15-48 |
| 7-15 | 10-20 | 20-40 | 6-10 | 10-15 | 15-* |
| 7-15 | 10-20 | 30-50 | 6-10 | 10-15 | 15-* |
| 7-15 | 10-20 | 40-60 | 6-10 | 10-15 | 15-* |
| 7-15 | 15-30 | 0-5 | 6-10 | 10-15 | 15-29 |
| 7-15 | 15-30 | 5-10 | 6-10 | 10-15 | 15-34 |
| 7-15 | 15-30 | 7-15 | 6-10 | 10-15 | 15-39 |
| 7-15 | 15-30 | 10-20 | 6-10 | 10-15 | 15-44 |
| 7-15 | 15-30 | 15-30 | 6-10 | 10-15 | 15-* |
| 7-15 | 15-30 | 20-40 | 6-10 | 10-15 | 15-* |
| 7-15 | 15-30 | 30-50 | 6-10 | 10-15 | 15-* |
| 7-15 | 15-30 | 40-60 | 6-10 | 10-15 | 15-* |
| 7-15 | 20-40 | 0-5 | 6-10 | 10-15 | 15-35 |
| 7-15 | 20-40 | 5-10 | 6-10 | 10-15 | 15-40 |
| 7-15 | 20-40 | 7-15 | 6-10 | 10-15 | 15-45 |
| 7-15 | 20-40 | 10-20 | 6-10 | 10-15 | 15-* |
| 7-15 | 20-40 | 15-30 | 6-10 | 10-15 | 15-* |
| 7-15 | 20-40 | 20-40 | 6-10 | 10-15 | 15-* |
| 7-15 | 20-40 | 30-50 | 6-10 | 10-15 | 15-* |
| 7-15 | 20-40 | 40-60 | 6-10 | 10-15 | 15-* |
| 7-15 | 30-50 | 0-5 | 6-10 | 10-15 | 15-41 |
| 7-15 | 30-50 | 5-10 | 6-10 | 10-15 | 15-* |
| 7-15 | 30-50 | 7-15 | 6-10 | 10-15 | 15-* |
| 7-15 | 30-50 | 10-20 | 6-10 | 10-15 | 15-* |
| 7-15 | 30-50 | 15-30 | 6-10 | 10-15 | 15-* |
| 7-15 | 30-50 | 20-40 | 6-10 | 10-15 | 15-* |
| 7-15 | 30-50 | 30-50 | 6-10 | 10-15 | 15-* |
| 7-15 | 30-50 | 40-60 | 6-10 | 10-15 | 15-23 |
| 7-15 | 40-60 | 0-5 | 6-10 | 10-15 | 15-47 |
| 7-15 | 40-60 | 5-10 | 6-10 | 10-15 | 15-* |
| 7-15 | 40-60 | 7-15 | 6-10 | 10-15 | 15-* |
| 7-15 | 40-60 | 10-20 | 6-10 | 10-15 | 15-* |
| 7-15 | 40-60 | 15-30 | 6-10 | 10-15 | 15-38 |
| 7-15 | 40-60 | 20-40 | 6-10 | 10-15 | 15-33 |
| 7-15 | 40-60 | 30-50 | 6-10 | 10-15 | 15-23 |
| 7-15 | 40-60 | 40-60 | 6-10 | 10-15 | 15-13 |
| 10-20 | 0-5 | 0-5 | 6-10 | 10-15 | 15-17 |
| 10-20 | 0-5 | 5-10 | 6-10 | 10-15 | 15-22 |
| 10-20 | 0-5 | 7-15 | 6-10 | 10-15 | 15-27 |
| 10-20 | 0-5 | 10-20 | 6-10 | 10-15 | 15-32 |
| 10-20 | 0-5 | 15-30 | 6-10 | 10-15 | 15-42 |
| 10-20 | 0-5 | 20-40 | 6-10 | 10-15 | 15-* |
| 10-20 | 0-5 | 30-50 | 6-10 | 10-15 | 15-* |
| 10-20 | 0-5 | 40-60 | 6-10 | 10-15 | 15-* |
| 10-20 | 5-10 | 0-5 | 6-10 | 10-15 | 15-* |
| 10-20 | 5-10 | 5-10 | 6-10 | 10-15 | 15-25 |
| 10-20 | 5-10 | 7-15 | 6-10 | 10-15 | 15-30 |
| 10-20 | 5-10 | 10-20 | 6-10 | 10-15 | 15-35 |
| 10-20 | 5-10 | 15-30 | 6-10 | 10-15 | 15-45 |
| 10-20 | 5-10 | 20-40 | 6-10 | 10-15 | 15-* |
| 10-20 | 5-10 | 30-50 | 6-10 | 10-15 | 15-* |
| 10-20 | 5-10 | 40-60 | 6-10 | 10-15 | 15-* |
| 10-20 | 7-15 | 0-5 | 6-10 | 10-15 | 15-23 |
| 10-20 | 7-15 | 5-10 | 6-10 | 10-15 | 15-28 |
| 10-20 | 7-15 | 7-15 | 6-10 | 10-15 | 15-33 |
| 10-20 | 7-15 | 10-20 | 6-10 | 10-15 | 15-38 |
| 10-20 | 7-15 | 15-30 | 6-10 | 10-15 | 15-48 |
| 10-20 | 7-15 | 20-40 | 6-10 | 10-15 | 15-* |
| 10-20 | 7-15 | 30-50 | 6-10 | 10-15 | 15-* |
| 10-20 | 7-15 | 40-60 | 6-10 | 10-15 | 15-* |
| 10-20 | 10-20 | 0-5 | 6-10 | 10-15 | 15-25 |
| 10-20 | 10-20 | 5-10 | 6-10 | 10-15 | 15-30 |
| 10-20 | 10-20 | 7-15 | 6-10 | 10-15 | 15-35 |
| 10-20 | 10-20 | 10-20 | 6-10 | 10-15 | 15-* |
| 10-20 | 10-20 | 15-30 | 6-10 | 10-15 | 15-* |
| 10-20 | 10-20 | 20-40 | 6-10 | 10-15 | 15-* |
| 10-20 | 10-20 | 30-50 | 6-10 | 10-15 | 15-* |
| 10-20 | 10-20 | 40-60 | 6-10 | 10-15 | 15-20 |
| 10-20 | 15-30 | 0-5 | 6-10 | 10-15 | 15-31 |
| 10-20 | 15-30 | 5-10 | 6-10 | 10-15 | 15-36 |
| 10-20 | 15-30 | 7-15 | 6-10 | 10-15 | 15-41 |
| 10-20 | 15-30 | 10-20 | 6-10 | 10-15 | 15-* |
| 10-20 | 15-30 | 15-30 | 6-10 | 10-15 | 15-* |
| 10-20 | 15-30 | 20-40 | 6-10 | 10-15 | 15-* |
| 10-20 | 15-30 | 30-50 | 6-10 | 10-15 | 15-* |
| 10-20 | 15-30 | 40-60 | 6-10 | 10-15 | 15-25 |
| 10-20 | 20-40 | 0-5 | 6-10 | 10-15 | 15-37 |
| 10-20 | 20-40 | 5-10 | 6-10 | 10-15 | 15-42 |
| 10-20 | 20-40 | 7-15 | 6-10 | 10-15 | 15-* |
| 10-20 | 20-40 | 10-20 | 6-10 | 10-15 | 15-* |
| 10-20 | 20-40 | 15-30 | 6-10 | 10-15 | 15-* |
| 10-20 | 20-40 | 20-40 | 6-10 | 10-15 | 15-* |
| 10-20 | 20-40 | 30-50 | 6-10 | 10-15 | 15-* |
| 10-20 | 20-40 | 40-60 | 6-10 | 10-15 | 15-20 |
| 10-20 | 30-50 | 0-5 | 6-10 | 10-15 | 15-43 |
| 10-20 | 30-50 | 5-10 | 6-10 | 10-15 | 15-* |
| 10-20 | 30-50 | 7-15 | 6-10 | 10-15 | 15-* |
| 10-20 | 30-50 | 10-20 | 6-10 | 10-15 | 15-* |
| 10-20 | 30-50 | 15-30 | 6-10 | 10-15 | 15-35 |
| 10-20 | 30-50 | 20-40 | 6-10 | 10-15 | 15-30 |
| 10-20 | 30-50 | 30-50 | 6-10 | 10-15 | 15-20 |
| 10-20 | 30-50 | 40-60 | 6-10 | 10-15 | 15-10 |
| 10-20 | 40-60 | 0-5 | 6-10 | 10-15 | 15-* |
| 10-20 | 40-60 | 5-10 | 6-10 | 10-15 | 15-* |
| 10-20 | 40-60 | 7-15 | 6-10 | 10-15 | 15-33 |
| 10-20 | 40-60 | 10-20 | 6-10 | 10-15 | 15-20 |
| 10-20 | 40-60 | 15-30 | 6-10 | 10-15 | 15-25 |
| 10-20 | 40-60 | 20-40 | 6-10 | 10-15 | 15-20 |
| 10-20 | 40-60 | 30-50 | 6-10 | 10-15 | 15-10 |
| 10-20 | 40-60 | 40-60 | 6-10 | 10-15 | 15-0 |
| 15-30 | 0-5 | 0-5 | 6-10 | 10-15 | 15-21 |
| 15-30 | 0-5 | 5-10 | 6-10 | 10-15 | 15-26 |
| 15-30 | 0-5 | 7-15 | 6-10 | 10-15 | 15-31 |
| 15-30 | 0-5 | 10-20 | 6-10 | 10-15 | 15-36 |
| 15-30 | 0-5 | 15-30 | 6-10 | 10-15 | 15-46 |
| 15-30 | 0-5 | 20-40 | 6-10 | 10-15 | 15-* |
| 15-30 | 0-5 | 30-50 | 6-10 | 10-15 | 15-* |
| 15-30 | 0-5 | 40-60 | 6-10 | 10-15 | 15-* |
| 15-30 | 5-10 | 0-5 | 6-10 | 10-15 | 15-* |
| 15-30 | 5-10 | 5-10 | 6-10 | 10-15 | 15-29 |
| 15-30 | 5-10 | 7-15 | 6-10 | 10-15 | 15-34 |
| 15-30 | 5-10 | 10-20 | 6-10 | 10-15 | 15-39 |
| 15-30 | 5-10 | 15-30 | 6-10 | 10-15 | 15-49 |
| 15-30 | 5-10 | 20-40 | 6-10 | 10-15 | 15-* |
| 15-30 | 5-10 | 30-50 | 6-10 | 10-15 | 15-* |
| 15-30 | 5-10 | 40-60 | 6-10 | 10-15 | 15-* |
| 15-30 | 7-15 | 0-5 | 6-10 | 10-15 | 15-27 |
| 15-30 | 7-15 | 5-10 | 6-10 | 10-15 | 15-32 |
| 15-30 | 7-15 | 7-15 | 6-10 | 10-15 | 15-37 |
| 15-30 | 7-15 | 10-20 | 6-10 | 10-15 | 15-42 |
| 15-30 | 7-15 | 15-30 | 6-10 | 10-15 | 15-* |
| 15-30 | 7-15 | 20-40 | 6-10 | 10-15 | 15-* |
| 15-30 | 7-15 | 30-50 | 6-10 | 10-15 | 15-* |
| 15-30 | 7-15 | 40-60 | 6-10 | 10-15 | 15-* |
| 15-30 | 10-20 | 0-5 | 6-10 | 10-15 | 15-30 |
| 15-30 | 10-20 | 5-10 | 6-10 | 10-15 | 15-35 |
| 15-30 | 10-20 | 7-15 | 6-10 | 10-15 | 15-40 |
| 15-30 | 10-20 | 10-20 | 6-10 | 10-15 | 15-45 |
| 15-30 | 10-20 | 15-30 | 6-10 | 10-15 | 15-* |
| 15-30 | 10-20 | 20-40 | 6-10 | 10-15 | 15-* |
| 15-30 | 10-20 | 30-50 | 6-10 | 10-15 | 15-* |
| 15-30 | 10-20 | 40-60 | 6-10 | 10-15 | 15-25 |
| 15-30 | 15-30 | 0-5 | 6-10 | 10-15 | 15-36 |
| 15-30 | 15-30 | 5-10 | 6-10 | 10-15 | 15-41 |
| 15-30 | 15-30 | 7-15 | 6-10 | 10-15 | 15-* |
| 15-30 | 15-30 | 10-20 | 6-10 | 10-15 | 15-* |
| 15-30 | 15-30 | 15-30 | 6-10 | 10-15 | 15-* |
| 15-30 | 15-30 | 20-40 | 6-10 | 10-15 | 15-* |
| 15-30 | 15-30 | 30-50 | 6-10 | 10-15 | 15-* |
| 15-30 | 15-30 | 40-60 | 6-10 | 10-15 | 15-* |
| 15-30 | 20-40 | 0-5 | 6-10 | 10-15 | 15-41 |
| 15-30 | 20-40 | 5-10 | 6-10 | 10-15 | 15-* |
| 15-30 | 20-40 | 7-15 | 6-10 | 10-15 | 15-* |
| 15-30 | 20-40 | 10-20 | 6-10 | 10-15 | 15-* |
| 15-30 | 20-40 | 15-30 | 6-10 | 10-15 | 15-* |
| 15-30 | 20-40 | 20-40 | 6-10 | 10-15 | 15-* |
| 15-30 | 20-40 | 30-50 | 6-10 | 10-15 | 15-* |
| 15-30 | 20-40 | 40-60 | 6-10 | 10-15 | 15-25 |
| 15-30 | 30-50 | 0-5 | 6-10 | 10-15 | 15-47 |
| 15-30 | 30-50 | 5-10 | 6-10 | 10-15 | 15-* |
| 15-30 | 30-50 | 7-15 | 6-10 | 10-15 | 15-* |
| 15-30 | 30-50 | 10-20 | 6-10 | 10-15 | 15-* |
| 15-30 | 30-50 | 15-30 | 6-10 | 10-15 | 15-* |
| 15-30 | 30-50 | 20-40 | 6-10 | 10-15 | 15-* |
| 15-30 | 30-50 | 30-50 | 6-10 | 10-15 | 15-25 |
| 15-30 | 30-50 | 40-60 | 6-10 | 10-15 | 15-15 |
| 15-30 | 40-60 | 0-5 | 6-10 | 10-15 | 15-* |
| 15-30 | 40-60 | 5-10 | 6-10 | 10-15 | 15-* |
| 15-30 | 40-60 | 7-15 | 6-10 | 10-15 | 15-38 |
| 15-30 | 40-60 | 10-20 | 6-10 | 10-15 | 15-25 |
| 15-30 | 40-60 | 15-30 | 6-10 | 10-15 | 15-30 |
| 15-30 | 40-60 | 20-40 | 6-10 | 10-15 | 15-25 |
| 15-30 | 40-60 | 30-50 | 6-10 | 10-15 | 15-15 |
| 15-30 | 40-60 | 40-60 | 6-10 | 10-15 | 15-5 |
| 20-40 | 0-5 | 0-5 | 6-10 | 10-15 | 15-25 |
| 20-40 | 0-5 | 5-10 | 6-10 | 10-15 | 15-30 |
| 20-40 | 0-5 | 7-15 | 6-10 | 10-15 | 15-35 |
| 20-40 | 0-5 | 10-20 | 6-10 | 10-15 | 15-40 |
| 20-40 | 0-5 | 15-30 | 6-10 | 10-15 | 15-50 |
| 20-40 | 0-5 | 20-40 | 6-10 | 10-15 | 15-* |
| 20-40 | 0-5 | 30-50 | 6-10 | 10-15 | 15-* |
| 20-40 | 0-5 | 40-60 | 6-10 | 10-15 | 15-* |
| 20-40 | 5-10 | 0-5 | 6-10 | 10-15 | 15-* |
| 20-40 | 5-10 | 5-10 | 6-10 | 10-15 | 15-33 |
| 20-40 | 5-10 | 7-15 | 6-10 | 10-15 | 15-38 |
| 20-40 | 5-10 | 10-20 | 6-10 | 10-15 | 15-43 |
| 20-40 | 5-10 | 15-30 | 6-10 | 10-15 | 15-* |
| 20-40 | 5-10 | 20-40 | 6-10 | 10-15 | 15-* |
| 20-40 | 5-10 | 30-50 | 6-10 | 10-15 | 15-* |
| 20-40 | 5-10 | 40-60 | 6-10 | 10-15 | 15-* |
| 20-40 | 7-15 | 0-5 | 6-10 | 10-15 | 15-31 |
| 20-40 | 7-15 | 5-10 | 6-10 | 10-15 | 15-36 |
| 20-40 | 7-15 | 7-15 | 6-10 | 10-15 | 15-41 |
| 20-40 | 7-15 | 10-20 | 6-10 | 10-15 | 15-* |
| 20-40 | 7-15 | 15-30 | 6-10 | 10-15 | 15-* |
| 20-40 | 7-15 | 20-40 | 6-10 | 10-15 | 15-* |
| 20-40 | 7-15 | 30-50 | 6-10 | 10-15 | 15-* |
| 20-40 | 10-20 | 0-5 | 6-10 | 10-15 | 15-* |
| 20-40 | 10-20 | 5-10 | 6-10 | 10-15 | 15-39 |
| 20-40 | 10-20 | 7-15 | 6-10 | 10-15 | 15-44 |
| 20-40 | 10-20 | 10-20 | 6-10 | 10-15 | 15-* |
| 20-40 | 10-20 | 15-30 | 6-10 | 10-15 | 15-* |
| 20-40 | 10-20 | 20-40 | 6-10 | 10-15 | 15-* |
| 20-40 | 10-20 | 30-50 | 6-10 | 10-15 | 15-* |
| 20-40 | 10-20 | 40-60 | 6-10 | 10-15 | 15-20 |
| 20-40 | 15-30 | 0-5 | 6-10 | 10-15 | 15-40 |
| 20-40 | 15-30 | 5-10 | 6-10 | 10-15 | 15-45 |
| 20-40 | 15-30 | 7-15 | 6-10 | 10-15 | 15-* |
| 20-40 | 15-30 | 10-20 | 6-10 | 10-15 | 15-* |
| 20-40 | 15-30 | 15-30 | 6-10 | 10-15 | 15-* |
| 20-40 | 15-30 | 20-40 | 6-10 | 10-15 | 15-* |
| 20-40 | 15-30 | 30-50 | 6-10 | 10-15 | 15-* |
| 20-40 | 20-40 | 0-5 | 6-10 | 10-15 | 15-46 |
| 20-40 | 20-40 | 5-10 | 6-10 | 10-15 | 15-* |
| 20-40 | 20-40 | 7-15 | 6-10 | 10-15 | 15-* |
| 20-40 | 20-40 | 10-20 | 6-10 | 10-15 | 15-* |
| 20-40 | 20-40 | 15-30 | 6-10 | 10-15 | 15-* |
| 20-40 | 20-40 | 20-40 | 6-10 | 10-15 | 15-* |
| 20-40 | 20-40 | 30-50 | 6-10 | 10-15 | 15-* |
| 20-40 | 20-40 | 40-60 | 6-10 | 10-15 | 15-20 |
| 20-40 | 30-50 | 0-5 | 6-10 | 10-15 | 15-* |
| 20-40 | 30-50 | 5-10 | 6-10 | 10-15 | 15-* |
| 20-40 | 30-50 | 7-15 | 6-10 | 10-15 | 15-* |
| 20-40 | 30-50 | 10-20 | 6-10 | 10-15 | 15-* |
| 20-40 | 30-50 | 15-30 | 6-10 | 10-15 | 15-35 |
| 20-40 | 30-50 | 20-40 | 6-10 | 10-15 | 15-30 |
| 20-40 | 30-50 | 30-50 | 6-10 | 10-15 | 15-20 |
| 20-40 | 30-50 | 40-60 | 6-10 | 10-15 | 15-10 |
| 20-40 | 40-60 | 0-5 | 6-10 | 10-15 | 15-* |
| 20-40 | 40-60 | 5-10 | 6-10 | 10-15 | 15-* |
| 20-40 | 40-60 | 7-15 | 6-10 | 10-15 | 15-33 |
| 20-40 | 40-60 | 10-20 | 6-10 | 10-15 | 15-20 |
| 20-40 | 40-60 | 15-30 | 6-10 | 10-15 | 15-25 |
| 20-40 | 40-60 | 20-40 | 6-10 | 10-15 | 15-20 |
| 20-40 | 40-60 | 30-50 | 6-10 | 10-15 | 15-10 |
| 30-50 | 0-5 | 0-5 | 6-10 | 10-15 | 15-30 |
| 30-50 | 0-5 | 5-10 | 6-10 | 10-15 | 15-35 |
| 30-50 | 0-5 | 7-15 | 6-10 | 10-15 | 15-40 |
| 30-50 | 0-5 | 10-20 | 6-10 | 10-15 | 15-45 |
| 30-50 | 0-5 | 15-30 | 6-10 | 10-15 | 15-* |
| 30-50 | 0-5 | 20-40 | 6-10 | 10-15 | 15-* |
| 30-50 | 0-5 | 30-50 | 6-10 | 10-15 | 15-* |
| 30-50 | 0-5 | 40-60 | 6-10 | 10-15 | 15-* |
| 30-50 | 5-10 | 0-5 | 6-10 | 10-15 | 15-33 |
| 30-50 | 5-10 | 5-10 | 6-10 | 10-15 | 15-38 |
| 30-50 | 5-10 | 7-15 | 6-10 | 10-15 | 15-43 |
| 30-50 | 5-10 | 10-20 | 6-10 | 10-15 | 15-* |
| 30-50 | 5-10 | 15-30 | 6-10 | 10-15 | 15-* |
| 30-50 | 5-10 | 20-40 | 6-10 | 10-15 | 15-* |
| 30-50 | 5-10 | 30-50 | 6-10 | 10-15 | 15-* |
| 30-50 | 5-10 | 40-60 | 6-10 | 10-15 | 15-20 |
| 30-50 | 7-15 | 0-5 | 6-10 | 10-15 | 15-36 |
| 30-50 | 7-15 | 5-10 | 6-10 | 10-15 | 15-41 |
| 30-50 | 7-15 | 7-15 | 6-10 | 10-15 | 15-* |
| 30-50 | 7-15 | 10-20 | 6-10 | 10-15 | 15-* |
| 30-50 | 7-15 | 15-30 | 6-10 | 10-15 | 15-* |
| 30-50 | 7-15 | 20-40 | 6-10 | 10-15 | 15-* |
| 30-50 | 7-15 | 30-50 | 6-10 | 10-15 | 15-* |
| 30-50 | 7-15 | 40-60 | 6-10 | 10-15 | 15-23 |
| 30-50 | 10-20 | 0-5 | 6-10 | 10-15 | 15-39 |
| 30-50 | 10-20 | 5-10 | 6-10 | 10-15 | 15-* |
| 30-50 | 10-20 | 7-15 | 6-10 | 10-15 | 15-* |
| 30-50 | 10-20 | 10-20 | 6-10 | 10-15 | 15-* |
| 30-50 | 10-20 | 15-30 | 6-10 | 10-15 | 15-35 |
| 30-50 | 10-20 | 20-40 | 6-10 | 10-15 | 15-30 |
| 30-50 | 10-20 | 30-50 | 6-10 | 10-15 | 15-20 |
| 30-50 | 10-20 | 40-60 | 6-10 | 10-15 | 15-10 |
| 30-50 | 15-30 | 0-5 | 6-10 | 10-15 | 15-44 |
| 30-50 | 15-30 | 5-10 | 6-10 | 10-15 | 15-* |
| 30-50 | 15-30 | 7-15 | 6-10 | 10-15 | 15-* |
| 30-50 | 15-30 | 10-20 | 6-10 | 10-15 | 15-* |
| 30-50 | 15-30 | 15-30 | 6-10 | 10-15 | 15-* |
| 30-50 | 15-30 | 20-40 | 6-10 | 10-15 | 15-* |
| 30-50 | 15-30 | 30-50 | 6-10 | 10-15 | 15-25 |
| 30-50 | 15-30 | 40-60 | 6-10 | 10-15 | 15-15 |
| 30-50 | 20-40 | 0-5 | 6-10 | 10-15 | 15-* |
| 30-50 | 20-40 | 5-10 | 6-10 | 10-15 | 15-* |
| 30-50 | 20-40 | 7-15 | 6-10 | 10-15 | 15-* |
| 30-50 | 20-40 | 10-20 | 6-10 | 10-15 | 15-* |
| 30-50 | 20-40 | 15-30 | 6-10 | 10-15 | 15-35 |
| 30-50 | 20-40 | 20-40 | 6-10 | 10-15 | 15-30 |
| 30-50 | 20-40 | 30-50 | 6-10 | 10-15 | 15-20 |
| 30-50 | 20-40 | 40-60 | 6-10 | 10-15 | 15-10 |
| 30-50 | 30-50 | 0-5 | 6-10 | 10-15 | 15-* |
| 30-50 | 30-50 | 5-10 | 6-10 | 10-15 | 15-* |
| 30-50 | 30-50 | 7-15 | 6-10 | 10-15 | 15-33 |
| 30-50 | 30-50 | 10-20 | 6-10 | 10-15 | 15-20 |
| 30-50 | 30-50 | 15-30 | 6-10 | 10-15 | 15-25 |
| 30-50 | 30-50 | 20-40 | 6-10 | 10-15 | 15-20 |
| 30-50 | 30-50 | 30-50 | 6-10 | 10-15 | 15-10 |
| 30-50 | 40-60 | 0-5 | 6-10 | 10-15 | 15-30 |
| 30-50 | 40-60 | 5-10 | 6-10 | 10-15 | 15-20 |
| 30-50 | 40-60 | 7-15 | 6-10 | 10-15 | 15-23 |
| 30-50 | 40-60 | 10-20 | 6-10 | 10-15 | 15-10 |
| 30-50 | 40-60 | 15-30 | 6-10 | 10-15 | 15-15 |
| 30-50 | 40-60 | 20-40 | 6-10 | 10-15 | 15-10 |
| 40-60 | 0-5 | 0-5 | 6-10 | 10-15 | 15-34 |
| 40-60 | 0-5 | 5-10 | 6-10 | 10-15 | 15-39 |
| 40-60 | 0-5 | 7-15 | 6-10 | 10-15 | 15-44 |
| 40-60 | 0-5 | 10-20 | 6-10 | 10-15 | 15-* |
| 40-60 | 0-5 | 15-30 | 6-10 | 10-15 | 15-* |
| 40-60 | 0-5 | 20-40 | 6-10 | 10-15 | 15-* |
| 40-60 | 0-5 | 30-50 | 6-10 | 10-15 | 15-* |
| 40-60 | 0-5 | 40-60 | 6-10 | 10-15 | 15-20 |
| 40-60 | 5-10 | 0-5 | 6-10 | 10-15 | 15-37 |
| 40-60 | 5-10 | 5-10 | 6-10 | 10-15 | 15-* |
| 40-60 | 5-10 | 7-15 | 6-10 | 10-15 | 15-* |
| 40-60 | 5-10 | 10-20 | 6-10 | 10-15 | 15-* |
| 40-60 | 5-10 | 15-30 | 6-10 | 10-15 | 15-35 |
| 40-60 | 5-10 | 20-40 | 6-10 | 10-15 | 15-30 |
| 40-60 | 5-10 | 30-50 | 6-10 | 10-15 | 15-20 |
| 40-60 | 5-10 | 40-60 | 6-10 | 10-15 | 15-10 |
| 40-60 | 7-15 | 0-5 | 6-10 | 10-15 | 15-40 |
| 40-60 | 7-15 | 5-10 | 6-10 | 10-15 | 15-* |
| 40-60 | 7-15 | 7-15 | 6-10 | 10-15 | 15-* |
| 40-60 | 7-15 | 10-20 | 6-10 | 10-15 | 15-* |
| 40-60 | 7-15 | 15-30 | 6-10 | 10-15 | 15-38 |
| 40-60 | 7-15 | 20-40 | 6-10 | 10-15 | 15-33 |
| 40-60 | 7-15 | 30-50 | 6-10 | 10-15 | 15-23 |
| 40-60 | 7-15 | 40-60 | 6-10 | 10-15 | 15-13 |
| 40-60 | 10-20 | 0-5 | 6-10 | 10-15 | 15-* |
| 40-60 | 10-20 | 5-10 | 6-10 | 10-15 | 15-* |
| 40-60 | 10-20 | 7-15 | 6-10 | 10-15 | 15-33 |
| 40-60 | 10-20 | 10-20 | 6-10 | 10-15 | 15-20 |
| 40-60 | 10-20 | 15-30 | 6-10 | 10-15 | 15-25 |
| 40-60 | 10-20 | 20-40 | 6-10 | 10-15 | 15-20 |
| 40-60 | 10-20 | 30-50 | 6-10 | 10-15 | 15-10 |
| 40-60 | 10-20 | 40-60 | 6-10 | 10-15 | 15-0 |
| 40-60 | 15-30 | 0-5 | 6-10 | 10-15 | 15-* |
| 40-60 | 15-30 | 5-10 | 6-10 | 10-15 | 15-* |
| 40-60 | 15-30 | 7-15 | 6-10 | 10-15 | 15-38 |
| 40-60 | 15-30 | 10-20 | 6-10 | 10-15 | 15-25 |
| 40-60 | 15-30 | 15-30 | 6-10 | 10-15 | 15-30 |
| 40-60 | 15-30 | 20-40 | 6-10 | 10-15 | 15-25 |
| 40-60 | 15-30 | 30-50 | 6-10 | 10-15 | 15-15 |
| 40-60 | 15-30 | 40-60 | 6-10 | 10-15 | 15-5 |
| 40-60 | 20-40 | 0-5 | 6-10 | 10-15 | 15-* |
| 40-60 | 20-40 | 5-10 | 6-10 | 10-15 | 15-* |
| 40-60 | 20-40 | 7-15 | 6-10 | 10-15 | 15-33 |
| 40-60 | 20-40 | 10-20 | 6-10 | 10-15 | 15-20 |
| 40-60 | 20-40 | 15-30 | 6-10 | 10-15 | 15-25 |
| 40-60 | 20-40 | 20-40 | 6-10 | 10-15 | 15-20 |
| 40-60 | 20-40 | 30-50 | 6-10 | 10-15 | 15-10 |
| 40-60 | 30-50 | 0-5 | 6-10 | 10-15 | 15-30 |
| 40-60 | 30-50 | 5-10 | 6-10 | 10-15 | 15-20 |
| 40-60 | 30-50 | 7-15 | 6-10 | 10-15 | 15-23 |
| 40-60 | 30-50 | 10-20 | 6-10 | 10-15 | 15-10 |
| 40-60 | 30-50 | 15-30 | 6-10 | 10-15 | 15-15 |
| 40-60 | 30-50 | 20-40 | 6-10 | 10-15 | 15-10 |
| 40-60 | 40-60 | 0-5 | 6-10 | 10-15 | 15-20 |
| 40-60 | 40-60 | 5-10 | 6-10 | 10-15 | 15-10 |
| 40-60 | 40-60 | 7-15 | 6-10 | 10-15 | 15-13 |
| 40-60 | 40-60 | 10-20 | 6-10 | 10-15 | 15-0 |

Superior Hydro Compositions

An excipient in present compositions optionally comprise water from about 5% to about 60% or more. It has been discovered that present compositions can optionally contain amounts of water and still retain superior properties (e.g., therapeutic, pharmacokinetic and pharmacodynamic properties). This is surprising in view of US Patent No. 20060067958 teaches that “water in the composition can retard the absorption rate and that the drug may not be soluble in the presence of water.

Moreover, local Drug doses can be attained that are similar to that which can be achieved by high alkanol formulations (for example, that of Example 8) wherein similar means, for example, 60%-150% or more). Without being bound by theory, additional amounts of water is believed to increase the hydrophilicity (and decrease hydrophobicity) of the composition thereby increasing the polar gradient between the topical composition and the more hydrophobic epidermis. Present compositions with optional water content described herein result in an increased polar gradient, faster diffusion of the Drug, resulting in greater Drug penetration.

It has surprisingly been discovered that present compositions comprising solvent systems taught herein have a superior capacity for containing water while maintaining single phasic and the Drug in solution. The amount of water that can be comprised by present compositions is greater than the amount that would be predicted by the capacity of the individual solvent alcohols containing a saturating amount of Drug (the “super solvent water effect”). Accordingly, compositions can now be made that are especially stable, for example, to “taking on water”, without causing the Drug to precipitate as might otherwise be expected. Moreover, high Drug compositions can now be made with greater amounts of water.

Illustrative examples of the surprising capacity of present compositions to comprise great amounts of water are set forth in Table 31 through Table 36 (and the accompanying Examples) Solvent systems that contain propylene glycol and polyethylene glycol generally show such an increase. Increased water capacity can be greater than 35%.

Water can now be added to the compositions in amounts of about 5% to about 20% or about 20% to about 40%, or about 40% to about 60% or more. Such compositions surprisingly have a superior property.

Compositions can now also be made with high concentrations of water and high concentrations of Drug. Such high Drug/high water compositions allow local delivery of greater amounts of Drug due to water enhanced diffusion, desirable thermodynamic properties, and a greater Drug concentration gradient between the applied composition at the surface of the sking and the target tissue. Additionally, Drug remains stable and does not readily precipitate in present compositions even when exposed to water (e.g. high relative humidity) due to the increased capacity of the formulation for water.

Examples of useful ranges of solvent alcohols, Drug, water, and excipients are shown in Table 2.

TABLE 2
Useful ranges of Hydro compositions

| PG | PEG 400 | EtOH | IPA | Water | Drug | Excipient |
|---|---|---|---|---|---|---|
| — | — | 15-25 | 15-25 | 10-20 | 30-40 | 0-30 |
| 20-25 | 15-20 | 0-10 | — | 15-25 | 14-30 | 0-36 |
| 20-25 | 15-20 | — | 5-10 | 20-30 | 15-30 | 0-25 |
| — | 20-30 | 5-10 | 5-10 | 20-25 | 20-35 | 0-30 |
| 25-35 | — | 5-10 | 5-10 | 20-30 | 20-35 | 0-25 |
| 15-25 | 10-15 | 0-5 | 0-10 | 20-30 | 15-35 | 0-40 |
| 28-30 | 21-22 | — | — | 26-33 | 15-25 | 0-10 |
| 38-40 | — | 9-10 | — | 25-30 | 20-28 | 0-8 |
| 37-40 | — | — | 10-11 | 24-30 | 20-28 | 0-9 |
| — | 35 | 8-15 | — | 24-29 | 24-29 | 0-5 |
| — | 33-35 | — | 12-13 | 27-28 | 24-28 | 0-4 |
| — | — | 20-22 | 22-24 | 9-17 | 40-47 | 0-9 |
| 24-25 | 18-19 | 6-10 | — | 25-31 | 19-27 | 0-8 |
| 23-25 | 17-19 | — | 6-7 | 24-31 | 19-29 | 0-11 |
| — | 26-28 | 7-10 | 8-15 | 21-26 | 28-35 | 0-6 |
| 29-32 | — | 7-8 | 8-9 | 22-28 | 24-33 | 0-10 |
| 20-22 | 15-16 | 5-10 | 6-10 | 22-29 | 22-33 | 0-10 |
| 20-30 | 20-30 | — | — | 15-40 | 5-30 | 0-40 |
| 20-30 | — | 15-25 | — | 15-40 | 10-30 | 0-40 |
| 10-20 | 40-60 | — | — | 10-25 | 10-25 | 0-30 |
| 40-60 | 10-20 | — | — | 10-25 | 10-25 | 0-30 |
| 15-25 | 25-30 | — | — | 25-35 | 10-25 | 0-25 |
| — | 30-40 | 5-10 | — | 25-30 | 20-30 | 0-20 |
| — | 30-40 | — | 5-15 | 20-30 | 15-30 | 0-30 |
| 25-35 | — | 5-15 | — | 20-30 | 20-30 | 0-30 |
| 25-35 | — | — | 10-15 | 20-30 | 20-30 | 0-25 |
| — | — | 15-25 | 15-25 | 10-20 | 30-40 | 0-30 |
| 15-20 | 20-25 | 0-10 | — | 15-25 | 14-30 | 0-36 |
| 15-20 | 20-25 | — | 5-10 | 20-30 | 15-30 | 0-25 |
| 20-30 | — | 5-10 | 5-10 | 20-25 | 20-35 | 0-30 |
| — | 25-35 | 5-10 | 5-10 | 20-30 | 20-35 | 0-25 |
| 10-15 | 15-25 | 0-5 | 0-10 | 20-30 | 15-35 | 0-40 |
| 21-22 | 28-30 | — | — | 26-33 | 15-25 | 0-10 |
| — | 38-40 | 9-10 | — | 25-30 | 20-28 | 0-8 |
| — | 37-40 | — | 10-11 | 24-30 | 20-28 | 0-9 |
| 30-37 | — | 10-15 | — | 24-29 | 24-29 | 0-5 |
| 33-35 | — | — | 12-13 | 27-28 | 24-28 | 0-4 |
| — | — | 20-22 | 22-24 | 9-17 | 40-47 | 0-9 |
| 18-19 | 24-25 | 6-10 | — | 25-31 | 19-27 | 0-8 |
| 17-19 | 23-25 | — | 6-7 | 24-31 | 19-29 | 0-11 |
| 26-28 | — | 7-10 | 8-15 | 21-26 | 28-35 | 0-6 |
| — | 29-32 | 7-8 | 8-9 | 22-28 | 24-33 | 0-10 |
| 15-16 | 20-22 | 5-10 | 6-10 | 22-29 | 22-33 | 0-10 |
| 20-30 | 20-30 | — | — | 15-40 | 5-30 | 0-40 |
| — | 20-30 | 15-25 | — | 15-40 | 10-30 | 0-40 |
| 0-10 | 0-20 | 0-30 | 0-20 | 30-50 | 5-15 | 0-65 |
| 2-10 | 30-55 | 10-30 | — | 5-25 | 5-15 | 0-48 |
| 2-10 | 30-55 | — | 10-30 | 5-25 | 5-15 | 0-53 |
| 0-10 | 20-30 | 15-30 | — | 20-50 | 5-15 | 0-40 |
| 0-10 | 20-30 | — | 15-30 | 20-50 | 5-15 | 0-40 |
| 25-30 | 15-25 | — | — | 25-35 | 10-25 | 0-25 |
| 30-40 | — | 5-10 | — | 25-30 | 20-30 | 0-20 |
| 30-40 | — | — | 5-15 | 20-30 | 15-30 | 0-30 |
| — | 25-35 | 5-15 | — | 20-30 | 20-30 | 0-30 |
| — | 25-35 | — | 10-15 | 20-30 | 20-30 | 0-25 |
| 20-25 | 15-20 | 0-10 | — | 15-25 | 5-15 | 0-45 |
| 20-25 | 15-20 | — | 5-10 | 20-30 | 5-15 | 0-35 |
| — | 20-30 | 5-10 | 5-10 | 20-25 | 5-15 | 0-45 |
| 25-35 | — | 5-10 | 5-10 | 20-30 | 5-15 | 0-40 |
| 15-25 | 10-15 | 0-5 | 0-10 | 20-30 | 5-15 | 0-50 |
| 28-30 | 21-22 | — | — | 26-33 | 5-15 | 0-20 |
| 38-40 | — | 9-10 | — | 25-30 | 5-15 | 0-23 |
| 37-40 | — | — | 10-11 | 24-30 | 5-15 | 0-24 |
| — | 35 | 8-15 | — | 24-29 | 5-15 | 0-24 |
| — | 33-35 | — | 12-13 | 27-28 | 5-15 | 0-23 |
| — | — | 20-22 | 22-24 | 9-17 | 5-15 | 0-44 |
| 24-25 | 18-19 | 6-10 | — | 25-31 | 5-15 | 0-8 |
| 23-25 | 17-19 | — | 6-7 | 24-31 | 5-15 | 0-22 |
| — | 26-28 | 7-10 | 8-15 | 21-26 | 5-15 | 0-29 |
| 29-32 | — | 7-8 | 8-9 | 22-28 | 5-15 | 0-29 |

Useful examplary hydro compositions are set forth in Table 3.

TABLE 3
Examplary Hydro Compositions

| PG | PEG | EtOH | IPA | Water | Drug |
|---|---|---|---|---|---|
| 28 | 21 | 0 | 0 | 26 | 25 |
| 38 | 0 | 9 | 0 | 25 | 28 |
| 37 | 0 | 0 | 10 | 24 | 28 |
| 0 | 35 | 12 | 0 | 24 | 29 |
| 0 | 33 | 0 | 12 | 27 | 28 |
| 0 | 0 | 22 | 24 | 9 | 44 |
| 24 | 18 | 6 | 0 | 25 | 27 |
| 23 | 17 | 0 | 6 | 24 | 29 |
| 0 | 26 | 9 | 10 | 21 | 35 |
| 29 | 0 | 7 | 8 | 22 | 33 |
| 20 | 15 | 5 | 6 | 22 | 33 |
| 30 | 22 | 0 | 0 | 33 | 15 |
| 40 | 0 | 10 | 0 | 30 | 20 |
| 40 | 0 | 0 | 11 | 30 | 20 |
| 0 | 35 | 12 | 0 | 29 | 24 |
| 0 | 35 | 0 | 13 | 28 | 24 |
| 0 | 0 | 20 | 22 | 17 | 40 |
| 25 | 19 | 6 | 0 | 31 | 19 |
| 25 | 19 | 0 | 7 | 31 | 19 |
| 0 | 28 | 9 | 10 | 26 | 28 |
| 32 | 0 | 8 | 9 | 28 | 24 |
| 22 | 16 | 5 | 6 | 29 | 22 |
| 10 | 20 | | | 47 | 15 |
| 2 | 20 | 15 | | 47 | 15 |
| 2 | 20 | 30 | | 32 | 15 |
| 2 | 30 | 30 | | 22 | 15 |
| 10 | 20 | | | 52 | 15 |
| 10 | | 20 | | 50 | 15 |
| 10 | | | 20 | 50 | 15 |
| 3 | 40 | 20 | | 15 | 15 |
| 3 | 30 | | 25 | 20 | 15 |
| 3 | 30 | | 20 | 25 | 15 |
| 2 | 27 | 27 | | 24 | 14 |
| 2 | 55 | 20 | | 2 | 15 |
| 2 | 30 | 20 | | 27 | 15 |
| 2 | 65 | 10 | | 2 | 15 |
| 2 | 50 | 10 | | 17 | 15 |
| 2 | 40 | 10 | | 27 | 15 |
| 24 | 18 | 0 | 0 | 27 | 12 |
| 32 | 0 | 8 | 0 | 24 | 16 |
| 32 | 0 | 0 | 9 | 24 | 16 |
| 0 | 28 | 9 | 0 | 24 | 19 |
| 0 | 28 | 0 | 10 | 23 | 19 |
| 0 | 0 | 16 | 18 | 14 | 32 |
| 20 | 15 | 5 | 0 | 25 | 15 |
| 20 | 15 | 0 | 5 | 25 | 15 |
| 0 | 22 | 7 | 8 | 21 | 22 |
| 25 | 0 | 6 | 7 | 22 | 19 |
| 17 | 13 | 4 | 5 | 23 | 17 |
| 23 | 17 | 0 | 0 | 20 | 20 |
| 30 | 0 | 8 | 0 | 20 | 23 |
| 30 | 0 | 0 | 8 | 19 | 22 |
| 0 | 28 | 9 | 0 | 19 | 24 |
| 0 | 27 | 0 | 10 | 21 | 22 |
| 0 | 0 | 18 | 20 | 7 | 36 |
| 19 | 14 | 5 | 0 | 20 | 22 |
| 18 | 14 | 0 | 5 | 19 | 23 |
| 0 | 21 | 7 | 8 | 17 | 28 |
| 24 | 0 | 6 | 6 | 18 | 26 |
| 16 | 12 | 4 | 4 | 18 | 26 |

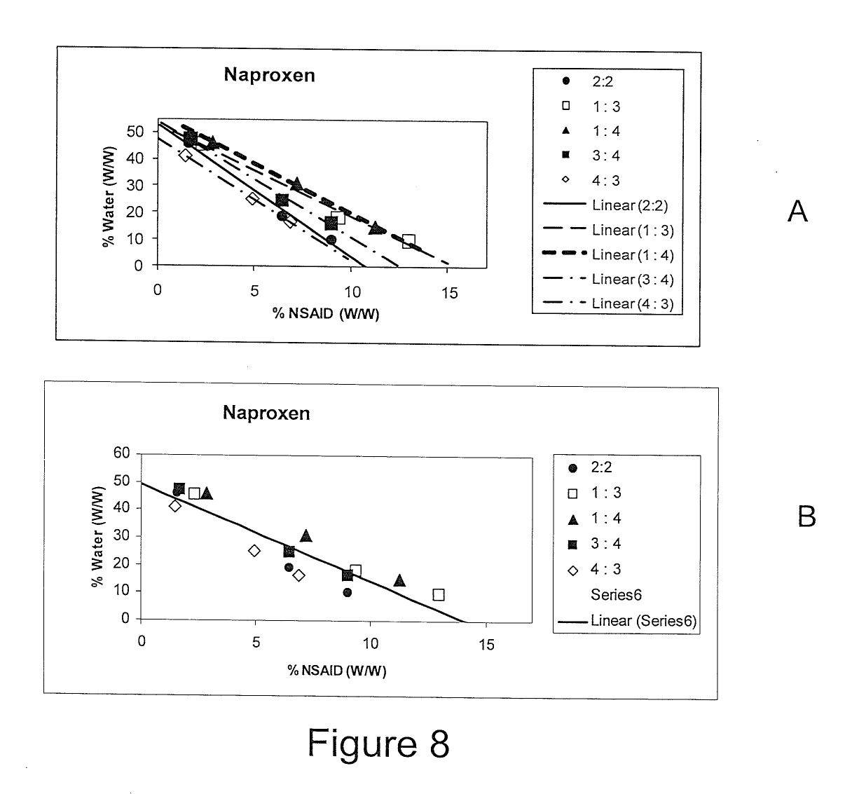

With the teaching herein, useful Drug and water concentrations can be determined for a given solvent system. Maximal Drug and water concentrations are determined, for example as set forth in FIG. 8 through FIG. 11. Such concentrations can be described by the equation W=mD+b, where W is a water concentration, D is the Drug concentration, b is the y intercept, and m is the slope.

Useful concentrations can be described by any of the following formulae:

W≦mD+b [Formula 10], or

W=mD+(0 to b) [Formula 11], or

W=m(0 to D)+b [Formula 12].

Low Alkanol Compositions.

In an optional embodiment, the solvent system is a low alkanol system comprising an alkanol and at least one of a polyethylene glycol (optionally having a molecular weight less than about 1100) or a propylene glycol. Surprisingly it has been discovered that such compositions of the present invention can comprise an alkanol but in an amount equal or less than about: 45% or 20% or 10% and still deliver local doses of the NSAID that are similar to high alkanol formulations (e.g. as set forth in Example 8).

As shall be readily apparent from the examples herein, in compositions where the Drug has a carboxylic acid group and where a solvent alcohol is a C-1 through C-3 straight chain alkanol (i.e. methanol, ethanol, or propanol), the alkanol and the Drug carboxylic acid group can react at a substantial rate to form an ester prodrug upon storage of the composition.

In compositions where the Drug has a carboxylic acid group and where a solvent alcohol is a branched alkanol or an alkanol with four or more carbons, the rate of ester formation between the alkanol and the carboxylic acids group upon storage is inhibited compared to a composition with a C-1 through C-3 straight chain alkanol.

Also, where the Drug has a carboxylic acid group and where a solvent alcohol is an alkanol, increasing the pH of the composition decreases the rate of formation of an ester between the alkanol and the carboxylic acid group upon storage. Lowering the pH increases the esterification rate. An esterification rate-stimulating pH is about 3.5 to about 5.0. An esterification rate inhibiting pH is above about 5 or about 6 or about 7.

Also, a lower concentration of alkanol in the composition decreases the rate of formation of an ester between the alkanol and the carboxylic acid group of the Drug upon storage,

Also as shall also be readily apparent from the examples herein, decreasing water concentration results in an increase in ester prodrug formation upon storage of a present composition. An esterification rate stimulating water concentration is below about 24% or below about 20% or below about 17%. An esterification rate inhibiting concentration of water is at or above about 24%, or above about 30% or above about 40%.

Optionally, present compositions comprising a Drug with a carboxylic acid group and where the solvent system contains a low amount of alkanol demonstrate superior stability upon storage—that is, a low rate of ester formation between the alkanol and the Drug. Examplary superior storage stability is where there is less than 1% of the Drug is esterified upon storage for one year at room temperature.

For example, present compositions with less than 20% alkanol demonstrate superior stability.

Present compositions can optionally be especially stable wherein the composition has less than about 20% alkanol, more than about 20% water, and a pH of about 5 to about 7. Especially stable compositions are ones that, for example, do not form about 1% NSAID alkanol ester in a year at room temperature or less than about 0.5%.

Low alkanol composition can comprise as little as about 5% or as much as about 45% alkanol (e.g., ethanol or isopropanol), about 10% to about 50% polyethylene glycol (having a molecular weight less than about 1100) or propylene glycol or mixtures thereof, and about 10% to about 50% water. The NSAID is present in an amount of about 50% to about 150% of the amount represented by the Formula 1 or according to any of Formula 2 through Formula 9

Examples of useful ranges can be found in low alkanol examples set forth in Table 1. Examples of useful compositions are set forth in Table 4, where NSAID I is 30% of the concentration based upon the maximum from Formula 2 through Formula 9 NSAID II is 100% of the maximum. The values in the excipient columns represent the amount that corresponds to the composition with NSAID II.

TABLE 4
Low Alkanol Compositions

| PG | PEG | EtOH | IPA | NSAID I | | NSAID II | Excipients |
|---|---|---|---|---|---|---|---|
| 10 | 30 | 10 | | 7.5 | to | 22.5 | 27.5 |
| 10 | 30 | 20 | | 10.8 | to | 32.5 | 7.5 |
| 30 | 10 | 10 | | 6.9 | to | 20.8 | 29.2 |
| 20 | 20 | 10 | | 7.2 | to | 21.7 | 28.3 |
| 20 | 20 | 20 | | 10.6 | to | 31.7 | 8.3 |
| 20 | 30 | 10 | | 8.3 | to | 25.0 | 15.0 |
| 10 | 30 | | 10 | 7.2 | to | 21.6 | 28.4 |
| 10 | 30 | | 20 | 10.2 | to | 30.7 | 9.3 |
| 30 | 10 | | 10 | 6.6 | to | 19.9 | 30.1 |
| 20 | 20 | | 10 | 6.9 | to | 20.8 | 29.2 |
| 20 | 20 | | 20 | 9.9 | to | 29.8 | 10.2 |
| 20 | 30 | | 10 | 8.0 | to | 24.1 | 15.9 |

In another embodiment, the present compositions are alkanol-free and have at least one of polyethylene glycol (having a molecular weight less than about 1100) or a propylene glycol.

Due, in part, to the superior solubility properties of present compositions with solvent systems taught here, high Drug, low alkanol compositions can optionally comprise water in greater amounts than might otherwise be predicted. Such alkanol-free, high Drug concentration, water containing compositions surprisingly result in delivery of local doses of the NSAID that are similar to high alkanol formulations (e.g. 50% or more). Examplary formulations with such properties are described elsewhere herein.

### Superior Properties

Compositions according to the present invention have one or more superior features desired in a topical formulation for a local disorder, namely (1) minimal systemic delivery; (2) rapid delivery of therapeutic levels of a Drug to the local target; (3) delivery of high levels of a Drug to the local target; (4) delivery of sustained therapeutic levels of the Drug for an extended period of time; (5) rheologic properties that increase skin exposure to the Drug; (6) increased Drug stability in the composition (e.g. decreased prodrug formation); and (7) other pharmacodynamic and pharmacokinetic properties.

With the present invention, it is now possible to prepare compositions with different pharmacodynamic and pharmacokinetic properties by selection of the NSAID and solvent system. Compositions of the present invention optionally provide one or more of the following superior features when compared to the same dose of NSAID administered orally:

(1) higher levels of drug in the local target tissue (e.g. skin, joints, or muscle);

(2) more sustained level of an NSAID in the local target tissue;

(3) more rapid delivery of an NSAID to the local target tissue;

(4) less systemic delivery

Without being bound by theory, the inventors believe that the present compositions provide an especially effective treatment for local inflammatory disorders because of, in part, the co-actions of a topically active drug, solvent alcohols in the solvent system, and optionally one or more excipients.

The Drug is solubilized in the solvent system and is able to partially diffuse through the hydrophobic epidermis. Evidence for diffusion is not only demonstrated by assays disclosed herein, but by a visual reduction in the amount of drug on the surface of the skin after the gel has penetrated the skin and/or dried (i.e. absence of “ashing”). Moreover, in some embodiments of the present invention, a prodrug is used with increased hydrophobicity (over its active metabolite). The inventors have discovered that such increased hydrophobicity enables increased, direct delivery of drug through the follicle opening to a specific therapeutic target (i.e. the epidermal lining of the follicular pore). In some inflammatory skin disorders such as PFB, this is a common site of injury.

The gel properties of the composition allows administration of an increased volume of composition (i.e. more thickly applied), especially when compared to liquid formulations. This provides higher doses of the topically active drug.

Optionally, components with an increased latent heat of vaporization when compared to alkanols, retard evaporation of the solvent system, allowing extended time for the Drug to be absorbed into the skin after application. For example, a latent heat of vaporization of the solvent/cosolvent system above 855 kJ.kg-1 provides for a useful drying time.

This is an improvement over formulations that evaporate quickly leaving greater amounts of the NSAID dried on the surface of the skin.

A high NSAID composition, when the NSAID is practically insoluble or poorly soluble in water, contains a high concentration of the solvent system, for example, about 10 to about 90% or for example more than about 20% or more than about 40% or more than about 60%.

The optional keratolytic agent removes the dead cells from the epidermis including regions around the hair follicles, sebaceous glands, and sweat glands. This facilitates entry of Drug into the epidermis directly by way of the follicles and also enhances diffusion of the drug through the epidermis.

The optional humectant draws water into the epidermis, follicles, and glands and causes them to open up. This co-action facilitating diffusion of the active drug to the therapeutic targets in skin.

The action of a keratolytic agent and/or a humectant in compositions of the present invention is especially beneficial in PFB, where the hair follicle is the site of the skin injury and, therefore, a therapeutic target.

Drug Concentrations

It has been surprisingly discovered that in present compositions, that high levels of drug can be achieved in target tissue due, in part, to the high concentration of the NSAID in the compositions. High drug concentration is, for example, greater that about: 5% or 10% or 15% or 20% or 25% or even 30%.

A technical feature of the present invention that contributes to the high level of drug that can be achieved in target tissue is that greater amounts of drug are solubilized in the solvent system. Moreover, higher concentrations of Drug in present compositions results in higher levels of Drug at the target tissue. This is surprising in view of Treffel et al. (British J of Derm 129:286-291, 1993) who show that rapid and high penetration of ibuprofen through the skin from hydroalcoholic gels do not follow Fick's law. Instead, a 10% ibuprofen had lower drug absorption than a 5% gel. Moreover, they show that when the solubility limit of the alcohol is exceeded, the drug precipitates and goes into a suspension, and remains as a solid film on the skin surface. Hence, Treffel et al teach away from high Drug, low alkanol compositions of the present invention and towards achieving superior pharmacokinetic properties by using high alcohol compositions and reducing the amount of ibuprofen to less than 10%.

### Solvent Alcohols and Solvent Systems

Solvent systems of the present invention comprise two or more solvent alcohols. Such solvent alcohols of the present invention are selected from topically acceptable, monohydric or polyhydric alcohols. Such solvent alcohols are well known in the art. They may be unsubstituted or substituted alkyl alcohols. They include, for example, ethanol, isopropyl alcohol, myristoyl alcohol, benzyl alcohol, a propylene glycol (e.g. propylene glycol and dipropylene glycol), polyether polyol (e.g. polyethylene glycol and its derivitives), glycerin and alkyl glycerol derivatives, polysorbate, sorbitol, and panthenol.

Acceptable solvent alcohols also include alkene glycols and polyalkylene glycols. Non-limiting examples include butylene glycol, propylene glycol, dipropylene glycol, polypropylene glycol, and polyethylene glycol and derivatives thereof.

Polyethylene glycols, optionally with average weights less than 1100 and any dermatological acceptable derivatives thereof are useful in the present compositions. Non-limiting examples include PEG 40 stearate, PEG 200 cocoate, PEG 200 monooleate, PEG 300 monooleate, PEG 300 monostearate, PEG 400 cocoate, PEG 400 dilaurate, PEG 400 dioleate, PEG 400 monolaurate, PEG 400 monooleate, PEG 400 monostearate, PEG 400 ricinoleate, PEG 600 dioleate, PEG 600 monolaurate. One skilled in the art will recognize that other selections of esterified substituents are suitable to form PEG derivatives that are useful in the present compositions.

The solvent systems described herein have a surprising effect on drug delivery of compositions of the present invention. Without being bound by theory, the inventors believe that NSAIDs are absorbed into the skin by two different mechanisms: diffusion from the solvent and transport concurrently with the solvent. Both mechanisms are competed with by evaporation of the solvent—especially in the case of volatile solvent alcohols. However, in high NSAID compositions, Drug is absorption through both mechanisms and can be substantially accelerated. Present compositions are believed to result in faster drug delivery, high drug levels at target sites, and deeper penetration. Nevertheless, the more hydrophilic nature of the dermis can result in the surprisingly minimal systemic delivery of NSAIDs in present compositions.

Solvent alcohols comprising the solvent system, are present in a total solvent amount of about 30% to about 80%, optionally from about 40% to about 70%, or optionally from about 50% to about 65%.

One such examplary formulation is 15% ibuprofen and comprising polyethylene glycol, propylene glycol, and water in ratio amount when compared to ibuprofen of about 1 to about 3, about .2 to about1.5 and about 2 to about 4, respectively.

### Drug

“Non-limiting examples of botanical agents useful as a Drug in present compositions are extracts of willow bark, turmeric root, licorice root, ginger root, boswsellia serrata, centella asiatica, duboisia leaf, galangal, green tea, oleanolic (olive leaf extract), oleuropein (olive leaf extract), rosemary, sandalwood seed (ximenynic acid), scutellaria root, and white birch bark. Other botanical agents include arjunolic acid, (glabridin), lupeol, rosmarinic acid, and ursolic acid. Other botanical agents include fenugreek (Trigonella foenum-graecum), feverfew (Tanacetum parthenium), san qi (Panax pseudoginseng notoginseng), german camomile (Matricaria recutita), liquorice (Glycyrrhiza glabra), yellow gentian (Gentiana lutea), siberian ginseng (Eleutherococcus senticosus), male fern (Dryopteris filixmas), thorn apple (Datura stramonium), yarrow (Achillea millefolium), wild yam (Dioscorea villosa), black cohosh (Cimicifuga racemosa), camomile (Chamaemelum nobile), horse chestnut (Aesculus hippocastanum), dang gui (Angelica sinensis), gotu kola (Centella asiatica), and resveratrol

In one embodiment, the Drug is an NSAID of the phenylacetic acid type such as 4-biphenylacetic acid, ibufenac, ibuprofen, ketoprofen, fenoprofen, fluribiprofen, Phenylacetic acid type NSAIDs are distinguished herein from phenylacetic acids that are di-substituted to form fused phenyl rings such as the naphhylene of naproxen.

In one embodiment, the Drug is an NSAID prodrug of the phenylacetic acid type is formed by an ester linkage to a pro-moiety at the hydroxyl group of the carboxylic acid.

In one embodiment, the Drug is an NSAID of the N-Arylanthranilic acid types such as the non-limiting examples mefenamic.

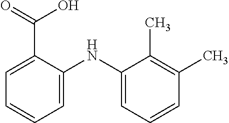

### Mefenamic Acid

In one embodiment, the Drug is an NSAID prodrug of the N-Arylanthranilic acid type is formed by an ester linkage to a pro-moiety at the hydroxyl group of the carboxylic acid.

In one embodiment, the Drug is an NSAID of the oxicam type such as the non-limiting examples piroxicam and meloxicam.

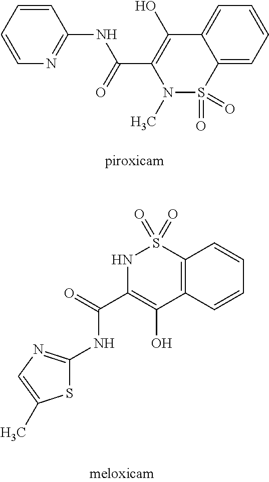

In one embodiment, the Drug is an NSAID prodrug of the oxicam type is formed by an ether linkage to a pro-moeity at the hydroxyl group of the fused ring heterocycle.

In one embodiment, the NSAID is diclofenac, indomethacin, and/or sulindac.

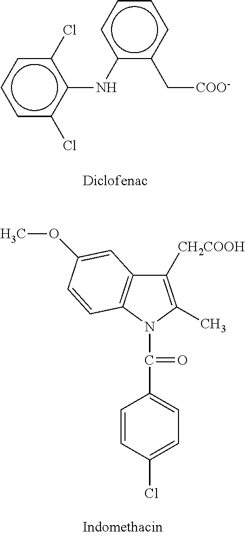

In one embodiment, the NSAID prodrug is formed by an ester linkage to a pro-moiety at the hydroxyl group of the carboxylic acid.

In one embodiment, the Drug is an NSAID prodrug of the naphthalene-acetic acid type exemplified by Naproxen. Optionally the naphthalene-acetic acid type NSAID prodrug is a C1-03 alkyl ester.

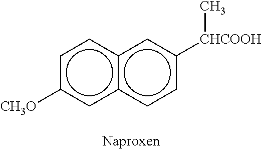

In one embodiment, the Drug is an NSAID prodrug of the naphthalene-acetic acid type formed by an ester linkage to a pro-moiety at the hydroxyl group of the carboxylic acid.

In one embodiment, the Drug is ketoprofen, ibuprofen, flurbiprofen, naproxen, acetaminophen, or diclofenac or salts, free acids, or esters thereof.

In one embodiment, the NSAID is a selective or preferential Cox-2 inhibitors. Illustrative examples of the COX-2 enzyme inhibitors that are advantageously administered by the present compositions include specific inhibitors such as celecoxib, valdecoxib, rofecoxib, varecoxib, parecoxib, and the like or preferential inhibitors such as meloxicam, nimesulide, etodolac, and the like.

In one embodiment, the NSAID is a macrolid such as tacrolimus and pimecrolimus.

In one embodiment, the NSAID is a bufexamac, dicoflenac, etofenamate, felbinac, entiazac, fepradinol, flufenamic, lunoxaprofen flubiprofen, ibuprofen, indomethacin, sonixin, ketoprofen, ketorolac, niflumic, oxyphenbutazone, piketoprofen, piroxicam, pranoprofen, or suxibuzone.

In one embodiment, the prodrug has an ester that can be formed by derivatizing a carboxylic acid.

In one embodiment, the Drug has a pKₐ from about 3.0 to about 6.5, optionally from about 4.3 to about 7, optionally from about 4 to about 5, optionally from about 4.2 to about 4.7, optionally about 4 to about 4.5, optionally from about 3.5 to about 4.5, and optionally form about 4.3 to about 4.5.

In one embodiment, the Drug has a has a log₁₀ P value of about 1.8 to about 5.5, optionally of about 3 to about 5, optionally of about 3 to about 4, optionally about 3.1 to about 3.6, optionally about 3.3 to about 3.7, optionally about 3.4 to about 3.6, optionally about 2.2 to about 2.6, optionally about 2.2 to about 2.4, and optionally of about 2 to about 3.

Table 5 sets forth examples of Drugs that satisify the optional limitations taught above with respect to pKa and to LogP.

TABLE 5
pKa and logP values of Drugs of present composition.

| Drug | pKa | Log P |
|---|---|---|
| piroxicam | 3.6 | |
| flufenamic acid | 3.65 | |
| mefenamic acid | 3.69 | |
| tiaprofenic acid | 4.05 | |
| flurbiprofen | 4.14 | |
| diclofenac | 4.18 | |
| indomethacin | 4.18 | |
| ketoprofen | 4.23 | |
| alclofenac | 4.26 | |
| felbinac | 4.29 | |
| naproxen | 4.4 | |
| ibuprofen | 4.41 | |
| tiaprofenic acid | 4.05 | |
| felbinac | 4.29 | |
| piroxicam | | 1.8 |
| alclofenac | | 2.47 |
| indomethacin | | 3.08 |
| ketoprofen | | 3.12 |
| naproxen | | 3.34 |
| ibuprofen | | 3.51 |
| flurbiprofen | | 4.16 |
| diclofenac | | 4.4 |
| mefenamic acid | | 5.12 |
| flufenamic acid | | 5.62 |

In one embodiment, the Drug is a botanical agent that is a herbal or botanical extract containing an anti-inflammatory component. The weight percent of the selected botanical agent in present compositions is adjusted according to the relative amount of anti-inflammatory component in the compound.

In one embodiment, the Drug is a prodrug of the ester type and is formed by reaction of an active drug of the present invention and an alcoholic solvent of the present composition. Optionally the Drug is a phenylacetic-type NSAID prodrug ester wherein pro-moiety is an amidyl, a thio, or an unsubstituted alkyl.

### Prodrug Compositions

It has been surprisingly discovered that when the Drug in a present composition is an NSAID prodrug, such a composition can have a superior drug delivery profile yet maintain reduced systemic delivery (for example, when compared to systemic levels of Drug after the same amount of Drug is administered orally or after the same amount of parent drug is delivered in the same composition). Without being bound by theory, it is believed that the hydrophobic nature of the NSAID prodrugs allows for superior dermal delivery. Such delivery is followed by release of the pro-moiety by resident enzymes in the skin (e.g. esterases), converting the prodrug to the less hydrophobic, parent drug. When conversion takes place in the epidermis, this less hydrophobic drug has reduced ability to diffuse through this more hydrophobic layer further to the more vascularized regions (e.g. the dermis). Any amount of prodrug that does diffuse into the more hydrophilic dermis is less mobile than its parent drug. Thus, this prodrug remains at this local target longer than would its parent drug. Examples of superior drug delivery profiles are: (1) greater local concentration of Drug; (2) longer half-life in the local tissue; (3) more rapid delivery; and (4) low circulating Drug level relative to local drug levels.

Compositions comprising NSAID prodrugs are especially useful for conditions where it is desirable to rapidly produce levels of an NSAID at a target site.

Compositions comprising NSAID prodrugs are especially useful for conditions where it is desirable to achieve high levels of Drug at the target tissues.

Compositions comprising such prodrugs can have reduced alkanol content at a given concentration of prodrug when compared to the corresponding parent drug. Because NSAID prodrugs generally have increased solubility in organic or hydrophobic solvent alcohols when compared to the corresponding parent NSAIDs, it is now possible to prepare a dermatologically acceptable composition with lower content of an alkanol solvent.

Examplary prodrugs of the present invention include NSAID pro-drugs, for example NSAID pro-drugs of the phenylacetic acid type. Other examplary NSAIDs and NSAID classes useful in the present invention are disclosed elsewhere herein. Those skilled in the art will readily recognize a functionality on a Drug that is useful for derivitization to add the “pro-moiety through a bond to the NSAID that can be processed in local tissues to form the parent drug.

Selection of the pro-moiety allows for modulation of dipole moment, charge, diffusion rate, and rate of hydrolytic cleavage to form the “parent” drug.

Prodrugs can be formed from a parent drug, for example, by adding a pro-moiety through esterification of a carboxylic acid functionality (for example, aryl carboxylic acid derivative NSAIDs). The hydrogen of the hydroxyl group of the carboxylic acid is replaced, for example by alky or aryl or carbonyl. An alkyl can be unsubstituted or substitiuted, for example, such as alkyloxyalkyl, alkoxycarbonylalkyl, alkoxycarbonylaminoalkyl, aminoalkyl, alkylcarbonylaminoalkyl,

Some examples of pro-moieties are methyl, ethyl, isopropyl, n-propyl, tert-butyl, butyl, pentyl, methoxy, tert-butoxy, methoxyethyl, ethoxymethyl, methoxymethyl, phenyl, carboxyethyl, methoxycarbonylmethyl, methoxycarbonylethyl, tert-butoxycarbonylaminomethyl, methoxycarbonyl, aminomethyl, and methylcarbonylaminomethyl; or a pharmaceutically-acceptable salt thereof.

A prodrug can also be produced to form an amide ester or a thioester.

A prodrug can be formed in an NSAID by, for example, adding a pro-moiety to the NSAID through ether formation at a hydroxyl functionality wherein the hydrogen of the hydroxyl functionality is replaced by an alkanoyloxyalkyl.

A pro-moiety can also be linked to an NSAID through formation of carbonates, carbamates, and amides covalently bonded through the carbonyl carbon.

Methods of preparation of prodrugs are described herein. Additional methods are described in, for example U.S. Pat. No. 5,073,641.

Additional methods are described in, for example U.S. Pat. No. 5,998,465.

Additional methods are described in, for example U.S. Pat. No. 5,811,438.

Additional methods are described in, for example U.S. Pat. No. 6,730,696.

Additional methods are described in, for example U.S. Pat. No. 6,620,813.

Additional methods are described in, for example U.S. Pat. No. 6,143,734.

Additional methods are described in, for example U.S. Pat. No. 5,750,564.

Additional methods are described in, for example U.S. Pat. No. 5,484,833.

Additional methods are described in, for example U.S. Pat. No. 5,315,027.

Additional methods are described in, for example U.S. Pat. No. 4,990,658.

Additional methods are described in, for example U.S. Pat. No. 4,851,426.

Additional methods are described in, for example U.S. Pat. No. 4,049,700.

Additional methods are described in, for example U.S. Pat. No. 3,228,831.

The above patent citations are hereby incorporated by reference in their entirety.

### Drug Combinations

It has been discovered a Drug in present compositions can be combined with other Drugs taught herein. For example, compositions of the present invention comprising an NSAID (non-prodrug type) and an NSAID of the prodrug type have surprisingly beneficial effects on local inflammatory disorders. Without being bound by theory, it is believed that the present compositions comprising an NSAID prodrug result in more rapid diffusion and greater localization than the corresponding parent NSAID. The prodrug, after being delivered to the target tissue, is converted to the parent NSAID. It is believed that conversion to the parent NSAID is not instantaneous upon absorption into the skin. It is also believed that the NSAID prodrug is not as active as the parent drug at the site of action. The NSAID in the composition generally provides a slower drug delivery as a result of the NSAIDs lower hydrophobicity but provides for higher activity once at a local site. Regardless of the mechanism, the NSAID prodrug/NSAID combination result in compositions with not only rapid and sustained delivery, but higher local concentration of active drug to target tissues.

### Drug—Solvent Alcohols Ratios

It has been discovered NSAIDs in present compositions can be solubilized at high concentrations. It has surprisingly been discovered that the amount of Drug that can be solubilized in the solvent system is greater than the sum of the individual amounts by at least 10% or optionally 20% or optionally 50% or more.

Composition Viscosity

Patient compliance with a recommended medical regimen (e.g. a patient regularly taking a medication as directed by his physician) is an important factor in therapeutic control of medical disorders. It has surprisingly been discovered that non-compliance is a bigger problem for subjects with a disorder that is non-life threatening or that is perceived by the subject as merely being a cosmetic problem (e.g. mild psoriasis, dermatitis, or PFB). Also surprising is the discovery that compliance can be improved when a composition has an especially pleasing skin feel. Accordingly, present composition can be made with different excipients (e.g. emollients and humectants) and at different viscosities to suit personal preferences among subjects—thereby increasing compliance and results in improved therapeutic control.

Viscosity values that are useful and desirable according to the present invention also vary as a function of the indication being treated. For example, where broad coverage (i.e. large areas of skin) or lower levels of drug application are desired, a less viscous composition is advantageous. Examples of less viscous compositions are about 1,000 cps to about 50,000 cps, or about 2,000 cps to about 25,000 cps, or 2,000 cps to about 10,000, or about 5,000 to about 15,000 cps. Such less viscous compositions facilitate spreading of applied composition.

Where more restricted coverage or higher levels of drug application are desired, a more viscous composition is advantageous. Examples of more viscous compositions are about 20,000 to about 200,000 or about 50,000 to about 100,000 cps.

The desired viscosity can be attained according to the present invention by selection of a dermatologically acceptable thickening agent and empirically determining the concentration necessary to achieve the desired thickening agent.

Certain solvent alcohols of the present invention, such as polyethylene glycol, can also be present at a viscosity-increasing amounts.

Optionally a present composition can further comprise a thickening agent such as a polymeric thickener comprising a homo- or copolymer having dissociable side groups on the polymer, such as acetic acid groups.

Optionally, the polymer is a polymer (or copolymer) of polyacrylic acids, such as those sold under the trade name CARBOPOL® (Noveon); polyoxyethylene-polyoxypropylene copolymers (Poloxamer) such as available as Lutrol, and the like. Carbopol®-type resins, such as Carbopol®, Pemulen® and Noveon®, are polymers of acrylic acid, crosslinked with polyalkenyl ethers or divinyl glycol. Carbopol®-type polymers are flocculated powders of particles averaging about 0.2 micron in diameter. Non-limiting examples of Carbopol® polymers are Carbopol® Ultrezrm 10, Carbopol®Ultrez™ 20, Carbopol® ETD™ 2020 and Carbopol® ETD™ 2001

Additional classes of polymers useful as a thickening agent according to the present invention are carboxyvinyl, polyacrylamides, polysaccharides, natural gums (for example, Xanthan Gum), polyvinlsulfonates, polyalkylsulfones and polyvinylalcohols or mixtures thereof may also be used.

Other classes of polymers useful according to the present invention are alkylcellulose materials, such as KLUCEL®, commercially available from Hercules (Wilmington, Del.).

Non-limiting examples of alkylcelluloses useful in the present invention include such as sodium carboxymethylcellulose, hydroxyethylcellulose, hydroxypropylcellulose, hydroxypropylmethylcellulose, and include methylcellulose.

Non-limiting examples of gums useful as thickening agents in the present invention include xanthan gum, sodium carageenan, sodium alginate, hydroxypropyl guar, gum Arabic (Acacia), and gum Tragacanth

In one embodiment, the polymeric thickener is present in compositions of the present invention at a total thickener amount of about 0.1% to about 5% of the total composition, optionally 0.5% to about 5%, or from about 1.5% to about 3% of the thickener component.

Present compositions have a viscosity in a range selected from the group of ranges consisting of about 2000 cps to about 200,000 cps, about 50,000 cps to about 200,000 cps, about 50,000 cps to about 100,000 cps, about 2,000 to about 50,000, about 2,000 cps to about 25,000 cps, about 2,000 cps to about 10,000 cps, and about 2,000 cps to about 5,000 cps.

### Composition pH

The compositions of the present invention generally have a pH of from about 3 to about 7, optionally from about 4.0 to about 5.5, or optionally from about 4.3 to about 5.0, or about 5 to about 7. One skilled in the art can readily determine useful dermatologically acceptable acids or bases to adjust composition pH.

### Low Alkanol Compositions

It has been discovered that optional compositions according to the present invention, have low alkanol concentration yet can have a high Drug concentration solubilized in the super solvent system. For example, super solvent systems can optionally comprise about 45% or less alkanol or less than about 30% or less than about 20% or less than about 10% or less than 5% or no alkanol.

Such low alkanol compositions are useful for local inflammatory disorders where alkanol is undesirable (e.g. conditions where a drying agent is contraindicated). Such undesirable conditions include conditions where it is undesirable to dry or further dry the skin. Examples of such disorders especially useful for treatment with a low alkanol compositions are psoriasis and dermatitis.

It has been discovered that low alkanol, high Drug compositions surprisingly demonstrate commercially acceptable Drug stability in such compositions. Despite alkanol concentration of 5% or more, formation of an alkanol ester is substantially reduced (compared to alcoholic gels, for example, the 60% ethanol gel of U.S. Pat. No. 5,093,133).

### Emolients

Emollients may be included in the compositions of the present invention for the purpose of enhancing both the formulation properties of the compositions (for example, the ability to apply the composition to the skin smoothly), as well as to provide desirable skin feel. Examples of such emollients include silicone materials, such as dimethicones (both cyclic and linear), pantethine derivatives (such as panthenol, pantothenic acid, pantetheine, and pantethine), and allantoin.

Emollients useful in instant compositions can be thin liquids, oils of various viscosities, fatty solids, or waxes. Hydrocarbons can function as an emollients by virtue of their ability to lubricate and/or hold water at the skin surface due to their relative occlusivity (e.g. mineral oil).

Emollients that are fatty chemicals, oily or waxy in nature, impart barrier properties (e.g. moisturizers) and encourage skin water retention. Suitable moisturizers and/or emollients in the skin disinfecting formulations include isopropyl palmitate, lanolin, derivatives of lanolin such as the ethoxylated acetylated alcohol and surface active alcohol derivatives of lanolin, propylene glycol, polypropylene glycol, polyethylene glycol, mineral oils, squalane, fatty alcohols, glycerin, and silicons such as dimethicone, cyclomethicone, simethicone.

Emollients and include one or more alcohol solvents making up the solvent system such as polyethylene glycol, glycerin, butylene glycol, diproylene glycol, and propylene glycol.

### Keratolytic Agents

The compositions of the present invention optionally include one or more keratolytic agents. Keratolytic agents used according to the invention may be chosen from α- and β-hydroxycarboxylic or β-ketocarboxylic acids, salts, amides or esters thereof. More particularly, non-limiting examples of a-hydroxy acids are glycolic acid, lactic acid, tartaric acid, malic acid, citric acid, mandelic acid and, in general, fruit acids. Non-limiting examples of β-hydroxy acids are salicylic acid and derivatives thereof, in particular alkyl derivatives, such as 5-n-octanoylsalicylic acid.

Keratolytic agent used according to the invention may also be chosen from retinoids (retinoic acid or retinol) and derivatives thereof, benzoyl peroxide, urea, boric acid, allantoin (e.g. glyoxyldiureide or 5-ureidohydantoin) sulfur, resorcinol, and hexachlorophene.

### Humectants

Optionally, compositions of the present invention comprise at least one humectant. Humectants useful according to the present invention are hygroscopic compounds that promote retention of water. Non-limiting examples of such are polyhydric alcohols (e.g. glycerin, propylene glycol, polypropylene glycol, mannitol and sorbitol, and the like) and polyols such as the polyethylene glycols, fructose, glucose, lactic acid, 1,3 butylene glycol, wheat gluten; macrocytis yyrifera; ceratonia silaqual; hespridin methyl chalocone; dipeptide-2; palmitoyl tetrpeptide-3; palmitoyl pentapeptides, and panthenols.

One or more humectants can optionally be included in the composition in total humectant amount of about 0.1% to about 20%, or about 0.5% to about 10%, or about 1% to about 5%.

### Miscellaneous Optional Components

The compositions of the present invention may also contain optional components which are typically used in topical pharmaceutical and/or cosmetic formulations. These materials, such as solvents, oils, emollients, surfactants, preservatives, colorants, UV blockers, and perfumes are well known in the art and they are used in the present compositions at their conventional art-established levels for their art-established effects.

Optionally, in other embodiments, it is advantageous to add antioxidants to the compositions of the invention. The antioxidants are advantageously selected from the group consisting of amino acids (e.g. glycine, histidine, tyrosine, tryptophan) and their derivatives, imidazoles, (e.g. urocanic acid) and their derivatives, peptides, such as D,L-carnosine, D-carnosine, L-carnosine and their derivatives (e.g. anserine), carotenoids, carotenes (e.g. .alpha.-carotene, .beta.-carotene, lycopene) and their derivatives, chlorogenic acid and derivatives thereof, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, .gamma.-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated closes (e.g. pmol to .mu.mol/kg), and also (metal) chelating agents (e.g. .alpha.-hydroxy fatty acids, palmitic acid, phytic acid, lactoferrin), .alpha.-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. .gamma.-linolenic acid, linoleic acid, oleic acid), folic acid and its derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and its derivatives, .alpha.-glucosylnitin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (e.g. ZnO, ZnSO.sub.4), selenium and its derivatives (e.g. selenomethionine), stilbenes and their derivatives (e.g. stilbene oxide, trans-stilbene oxide), vitamin A, vitamin B2, vitamin B6, vitamin B9 (folic acid), vitamin B12, vitamin C, vitamin E, selenium, carotenes (beta-carotene, lutein, zeaxanthin, and lycopene), zinc, copper, proanthocyanidins (e.g. anthocyanidins, flavonols [e.g. catechins, epicatechins, procyanidins], flavanones, flavonols), nac n-acetylcysteine, alpha-lipoic acid, coenzyme q10, ginkgo biloba, green tea extract, isothiocyanates (e.g. sulforaphane), phenols (e.g. caffeic acid, and ferulic acid), sulfides/thiols (e.g. diallyl sulfide, allyl methyl trisulfide, and dithiolthiones), lycopenes, and the derivatives (salts, esters, ethers, sugars, nucleotides, nucleosides, peptides and lipids) of said active ingredients which are suitable according to the invention.

The amount of antioxidants (one or more compounds) in the compositions is in an amount of from about 0.001% to about 30%, or from about 0.05% to about 20%, or about 1% to about 10%.

If vitamin E and/or its derivatives are used as the antioxidant or antioxidants, their respective concentrations are advantageously chosen from the range of about 0.001 to about 10%.

If vitamin A or vitamin A derivatives or carotenes or their derivatives are used as the antioxidant or antioxidants, their respective concentrations are advantageously chosen from the range of about 0.001 to about 10%.

The compositions may also contain oils, generally at levels of from about 0% to about 5% of the composition. The oils may be present for their emollient effects or can be used as part of an oil/water emulsion composition. The oils which may be used in the present invention are generally partially or poorly soluble in C₈ or greater alcohols. Examples of such oils include mineral oils, safflower oil, castor oil, sunflower oil, silicone oil, olive oil, dimethicone, cyclomethicone, triglycerides, particularly preferred is dimethicone.

The compositions of the present invention may also contain surfactants which generally act to improve the formulation properties of the compositions. Typically, surfactants are included at a concentration of about 0% to about 5% of the composition. Nonionic surfactants are generally the ones used in the present invention, with sorbitol fatty acid esters and alkyl polyethoxylates (for example, C₈-C₁₈ (EO)₄₋₅₀) being preferred. Examples of surfactants which may be utilized in the present invention include polysorbate 20 and polysorbate 80, both of which have commercial availability.

Optionally, embodiments of the present invention further comprise a UV-absorbing agent (UV blocker).

The composition of the invention may further comprise penetration enhancers for improved transepidermal or percutaneous delivery of drug. Examplary penetration enhancers suitable for the present invention include terpenes, terpene alcohols, essential oils, surfactants, and the like. Some such examples include d-limonene, terpinen-4-ol, menthone, 1,8-cineole, 1-pinene, .alpha terpineol, carveol, carvone, pulegone, eucalyptol, peppermint oil, sorbitan esters, polysorbates, sodium lauryl sulphate, and the like. Present compositions that comprise a penetration can achieve the desired therapeutic levels yet formulated with a reduced concentration of Drug, Moreover, using solvent systems taught herein with one or more penetration enhancers and near saturating concentrations of a Drug, higher levels of Drug are deliverable to target tissues.

Present compositions can optionally further comprise one, two, three, or four of the four of the following:
Glycerin (about 0.1 to 15%)
Panthenol (about 0.1 to 15%)
Polysorbate (about 0.1 to 15%)
Humectant (about 0.1% to about 20%)

Useful compositions are set forth in Table 6.

TABLE 6
Present Composition Ranges

| | | |
|---|---|---|
| Drug | 5-40 | 15-30 |
| Solvent system | 5-95 | 30-60 |
| Glycerin | 0-5 | 0-15 |
| Thickener | 0-5 | 0-5 |
| Water | 0-80 | 0-40 |
| Glycerin | 0-5 | 0-15 |
| Keratolytic (e.g. | 0-5 | 0-15 |
| Salicylic Acid) | | |
| Polysorbate | 0-5 | 0-5 |
| Propylene | 0-5 | 0-15 |
| Glycol | | |
| Panthenol | 0-2 | 0-2 |
| Emollients, | 0-40 | |
| humectants, | | |
| counterirritants | | |
| preservatives | 0-2 | |

### Optional Active Agent

Optionally, present compositions further comprise a second drug (e.g. a Drug or an active agent other than a Drug).

UV Blockers.

UV sunscreens can be used in combination with a Drug in present compositions. By “UV-A and/or UV-B sunscreen” means any compound or any combination of compounds which, by mechanisms that are known per se of absorption and/or reflection and/or scattering of UV-A and/or UV-B radiation, prevents, or at least limits, the contact between such radiation and a surface (skin, hair) on which this or these compounds have been applied. Stated differently, these compounds may be UV-absorbing organic screening agents or inorganic (nano) pigments which scatter and/or reflect UV radiation, as well as mixtures thereof.

According to the present invention, the at least one UV-A and/or UV-B sunscreen may comprise one or more hydrophilic organic screening agents and/or one or more lipophilic organic screening agents and/or one or more mineral or inorganic (nano)pigments.

UV blockers can be selected from, for example, singular (monomeric) aromatic compounds and/or reflecting pigments such as octyl methoxycinnamate (Parsol MCX), benzophenone-3(Oxybenzone) and octyl dimethyl PABA.

UV-photoprotecting agent according to the present invention can be dibenzoylmethane sunscreen avobenzone, or 4-(tert-butyl)-4′-methoxydibenzoylmethane, which is very well known to this art, is commercially available and is marketed, for example, under the trademark “PARSOL 1789” by Givaudan.

Sunscreens according to the present invention which are physical blockers reflect or scatter ultraviolet radiation. Typical examples of physical blockers include red petrolatum, titanium dioxide, and zinc oxide. These physical blockers have been employed in a variety of suspensions and particle sizes and are frequently included in cosmetic formulations. A review of physical blockers may be found at “Sun Protection Effect of Nonorganic Materials,” by S. Nakada & H. Konishi, Fragrance Journal, Volume 15, pages 64-70 (1987), which is incorporated by reference herein.

Sunscreens according to this invention which are chemical absorbers, like avobenzone, actually absorb harmful ultraviolet radiation. It is well known that chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties which are discussed at length in “Sunscreens—Development, Evaluation and Regulatory Aspects,” by N. Shaath et al., 2nd. Edition, pages 269-273, Marcel Dekker, Inc. (1997). This discussion, in its entirety, is incorporated by reference herein.

The sunscreens which may be formulated according to the present invention typically comprise chemical absorbers, but may also comprise physical blockers. Examplary sunscreens which may be formulated into the compositions of the present invention are chemical absorbers such as p-aminobenzoic acid derivatives, anthranilates, benzophenones, camphor derivatives, cinnamic derivatives, dibenzoyl methanes, .beta.-diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also expressly incorporated by reference. Also examplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. No. 5,087,445, issued to Haffey et al. on Feb. 11, 1992; U.S. Pat. No. 5,073,372, issued to Turner et al. on Dec. 17, 1991; and Chapter VIII of Cosmetics and Science and Technology by Segarin et al., pages 189 et seq. (1957), all of which are incorporated herein by reference in their entirety.

Sunscreens which may be formulated into the compositions of the present invention are those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N, N, N-trimethyl-4-(2-oxoborn-3-ylidene methyl)anillinium methyl sulfate, and mixtures thereof.

Similarly preferred sunscreens active in the UV-A and/or UV-B range include: p-aminobenzoic acid, oxyethylene (25 mol) p-aminobenzoate, 2-ethylhexyl p-dimethylaminobenzoate, ethyl N-oxypropylene p-aminobenzoate, gycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammunium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, .beta.(2-oxoborn-3-ylidene)-tolyl-4-sulfonic acid and soluble salts thereof, 3-(4′-sulfo)benzyliden-bornan-2-one and soluble salts thereof, 3-(4′methylbenzylidene)-d,1-camphor, 3-benzylidene-d,1-camphor, benzene 1,4-di(3-methylidene-10-camphosulfonic) acid and salts thereof (the product Mexoryl SX described in U.S. Pat. No. 4,585,597 issued to Lange et al. on Apr. 29, 1986), urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, 2,4-bis{[4-(2-ethyl-hexyloxy)]-2-hydroxy]-phenyl}-6-(4-methoxyphenyl)-1,3,5 -triazine (“TINOSORB S” marketed by Ciba), the polymer of N-(2 et 4)-[(2-oxoborn-3-yliden)methyl]benzyl]-acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (Drometrizole Trisiloxane), dispersed 2,2′-methylene-bis-[6-(2H-benzotriazol-2-yl)-4-(1,1,3,3-tetramethylbutyl)phenol]such as that marketed under the trademark MIXXIM BB/100 by Fairmount Chemical, or micronized in dispersed form thereof such as that marketed under the trademark TINOSORB M by Ciba-Geigy, and solubilized 2,2′-methylene-bis-[6-(2H-benzotriazol-2-yl)-4-(methyl)phenol] such as that marketed under the trademark MIXXIM BB/200 by Fairmount Chemical.

Examplary sunscreens are one or more of the following: octyl salicylate, octocrylene, and oxybenzone. Combinations of one of more of these sunscreens are also useful.

The dibenzoyl methane derivatives other than avobenzone are also preferred sunscreens according to the present invention. These are described, for example, in FR-2,326,405, FR-2,440,933 and EP-0,114,607, hereby expressly incorporated by reference.

Non-limiting examples of dibenzoyl methane sunscreens other than avobenzone include (whether singly or in any combination):, 2-methyldibenzoylmethane, 4-methyldibenzoylmethane, 4-isopropyldibenzoylmethane, 4-tert.-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert. -butyl-4′-methoxydibenzoylmethane, 2,4-dimethyl-4′-methoxydibenzoylmethane, 2,6-dimethyl-4-tert.-butyl-4′-methoxydibenzoylmethane, The subject at least one UV-A and/or UV-B sunscreen is advantageously formulated into the compositions of the invention in amounts ranging from about 0.01% to about 10%, and preferably from about 0.1% to about 6%, by weight thereof. Of course, depending upon the nature of the particular formulation, higher or lower amounts may be suitable.

The judiciously selected polymeric film former tricontanyl PVP is also well known to this art and it too is available commercially. Tricontanyl PVP is a copolymer of vinyl pyrrolidone and 1-triacontane. It is marketed in the United States by International Specialty Products (“ISP”) under the trademark “Ganex WP-660” and in Europe, also by ISP, under the trademark “Antaron WP-660.”

The concentration of the tricontanyl PVP polymer formulated into the compositions of the invention advantageously ranges from about 1% to about 10%, preferably from about 1% to 5%, by weight thereof. Also depending upon the nature of the particular formulation, higher or lower amounts may be suitable.

Antibiotics

Antibiotics can be used in combination with a Drug in present compositions.

Antibiotics of the present application can be protein synthesis inhibiting, or non-protein synthesis inhibiting antibiotics. The terminology “protein synthesis inhibiting antibiotic” means an agent that disrupts the bacterial ribosome cycle through which polypeptide chain initiation and elongation is normally effected. There are multiple points in the ribosome cycle at which this can occur.

The terminology “non-protein synthesis inhibiting antibiotic” means antibiotics other than protein synthesis inhibiting antibiotics.

As non-limiting representative examples of “protein synthesis inhibiting antibiotics” there may be mentioned: the aminoglycosides such as streptomycin, amikacin, and tobramycin; the macrolides such as erythromycin, clarithromycin, and lincomycin; the tetracyclines such as tetracycline, doxycycline, chlortetracycline, and minocycline; the oxaxolidinones such as linezolid; fusidic acid; and chloramphenicol.

As non-limiting representative examples of “non-protein synthesis inhibiting antibiotics” there may be mentioned: the beta-lactam penicillins such as penicillin, amoxicillin, dicloxacillin, and ampicillin; the beta lactam cephalsporins such as cefotaxime, cefuroxime, cefaclor, and ceftriaxone; the beta lactam carbapenems such as imipenem and meropenem; the quinolones such as ciprofloxacin, moxifloxacin, and levofloxacin; the sulfonamides such as sulfanilimide and sulfamethoxazole; metronidazole; rifampin; vancomycin; and nitrofurantoin. The following are examples of some antibiotics with half-lives of about 1 to 12 hours: Cefadroxil, cefazolin, cephalexin, cephalothin, cephapirin, cephacelor, cephprozil, cephadrine, cefamandole, cefonicid, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, loracarbef, imipenem, erythromycin (and erythromycin salts such as estolate, ethylsuccinate, gluceptate, lactobionate, stearate), azithromycin, clarithromycoin, dirithromycin, troleanomycin, penicillin V, peniciluin salts, and complexes, methicillin, nafcillin, oxacillin, cloxacillin, dicloxacillin, amoxicillin, amoxicillin and clavulanate potassium, ampicillin, bacampicillin, carbenicillin indanyl sodium (and other salts of carbenicillin) mezlocillin, piperacillin, piperacillin and taxobactam, ticarcillin, ticarcillin and clavulanate potassium, clindamycin, vancomycin, novobiocin, aminosalicylic acid, capreomycin, cycloserine, ethambutol HC1 and other salts, ethionamide, and isoniazid, ciprofloxacin, levofloxacin, lomefloxacin, nalidixic acid, norfloxacin, ofloxacin, sparfloxacin, sulfacytine, suflamerazine, sulfamethazine, sulfamethixole, sulfasalazine, sulfisoxazole, sulfapyrizine, sulfadiazine, sulfamethoxazole, sulfapyridine, metronidazole, methenamine, fosfomycin, nitrofurantoin, trimethoprim, clofazimine, co-triamoxazole, pentamidine, and trimetrexate.

Antifungal Agents

Antifungal agents can be used in combination with a Drug in present compositions. Non-limiting examples of antifungal agents of the present invention include amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc pyrithione and sodium pyrithione, butenafine, butoconazole, clioquinol, itraconazole, lanoconazole, neticonazole, tioconazole, terconazole, ciclopirox olamine, lactic acid, sorbic acid, cinnamic aldehyde or pharmaceutically acceptable salts or derivatives of any one of the aforementioned. The antifungal agent may be a Non-imidazole bearing antifungal agent (i.e. lacking an imidazole functional group within the molecule). Non-limiting examples include benzylamine-containing antifungal agents, for example butenafine, or allylamine-containing antifungal agents such as terbinafine, naftifine, and the like.

Antipsoriatic Agents

Antpsoriaticl agents can be used in combination with a Drug in present compositions The antipsoriatic agents are preferably alclometasone, amcinonide, betamethasone, clobetasol, clocortolone, desonide, desoximetasone, diflorasone, fluocinolone, fluocinonide, flurandrenolide, halcinonide, hydrocortisone, mometasone, prednicarbate, triamcinolone, salts thereof, derivetives thereof, and mixtures thereof. Preferably the concentration of the antipsoriatic agents in the varnish solution is in the range from about 0.02% to about 2% (w/w) and most preferably 0.2-1.5% (w/w). Preferably the concentration of the antipsoriatic agents based on the weight of the non-volatile components is in the range from about 0.1% to about 10% (w/w) and most preferably 1-7.5% (w/w).

### Combination Therapy

Present compositions optionally contain a UVA and/or UVB sunscreen (or “blocker”). It has surprisingly been discovered that such combination (i.e. NSAID plus UVA and/or UVB blocker) is especially beneficial to inflammatory skin disorders. Without being bound by theory, damage from UVA and UVB can cause a prostaglandin mediated inflammatory response and that such a response preconditions or “primes” subjects prone to inflammatory skin conditions. Concurrent or subsequent stimuli of a local inflammatory response (e.g. an irritant for contact dermatitis or hair penetration of the skin for PFB) can then cause an amplified response. Moreover, in mild forms of skin inflammatory disorders, UV damage can cause a response that otherwise might be subclinical.

Optionally, present compositions are formulated with antibacterial or antifungal drugs. In certain skin disorders, the initial stimulus is an invasion of the skin by an infectious agent. The skin, in response, can mount an inflammatory response. Such an inflammatory response is commonly understood as a defensive response. Without being bound by theory, it is believed by the present inventors that in certain cases, such an inflammatory response contributes to progression of the skin infection. Accordingly, it has surprisingly been discovered that skin infections can sometimes be better managed (quicker improvement, more limited severity, less spread, etc) by combining a present Drug and an antifungal or antibacterial drug in a present composition.

For example, a combination of a Drug and an antifungal agent (e.g. ketoconazol or tumeric oil) in a present composition can have unexpectedly advantageous therapeutic control in dermatophytoses. Such compositions can also have unexpectedly advantageous therapeutic control in seborrheic dermatitis and pityriasis capitis.

For example, a combination of a Drug (e.g. one or more NSAIDs such as ketoprofen, acetaminophen, ibuprofen, flurbiprofen, naproxen, or diclofenac) and triazole fluconazole can have unexpectedly advantageous therapeutic control in dermatophytoses. Useful optional additions to such combinations are additional antifungal agents such as allylamine terbinafine and/or an antibacterial soothing oil (e.g., tumeric oil extract). Folliic acid (or derivatives or salts) also is a useful addition. Such compositions offer superior control for chronic fungal infections (e.g. tinea pedis [athlete's foot] and tinea cruris [jock itch]). Because these conditions are readily transmitted by the use of communal showering and bathing facilities, close quartering of personnel and warm moist climates, chronic treatment is often required. Thus, such present compositions are especially useful because of superior safety profiles, in addition to the other advantages taught herein.

Especially useful combinations of antifungal agents are those which provide two or more different antifungal agents with different mechanisms of action. For example, present compositions containing ibuprofen and ketoconazole provide antifungal protection by damaging fungal cytoplasmic membranes and inhibiting ergosterol biosynthesis. It has been discovered herein that antifungal efficacy can thus be enhanced by the co-action of such two different mechanisms. Without being bound by theory, it is believed herein that fungi that escape cytoplasmic damage are more readily killed by an inhibition of ergosterol biosynthesis. Similarly, fungi that escape inhibition of ergosterol biosynthesis are more readily killed by cytoplasmic damage. Thus, this escape and capture phenomenon can be effectively achieved through the co-action of a combination of other antifungal agents taught herein. For example, selection of two such antifungal agents can be aided by Table 7 below.

TABLE 7
Antifungal Mechanisms of Action

| Antifungal Drug Class | Examples | Mechanism of Action |
|---|---|---|
| Polyenes | Amphotericin | Interacts w/ sterols in cell membranes |
| | B Nystatin | forming channels that leak cellular |
| | | contents |
| Antibiotic | Griseofulvin | Inhibits mitosis (sliding of microtubules) in |
| | | fungi |
| Azoles | Fluconazole, | Inhibit ergosterol biosynthesis at the level |
| | Itraconazole, | of C14-demethylase |
| | Ketoconazole, | |
| | etc. | |
| Allylamines | Terbinafine | Inhibit ergosterol biosynthesis at the level |
| | | of squalene epoxidase |
| Thiocarbamate | Tolnaftate | Inhibit ergosterol biosynthesis at the level |
| | | of squalene epoxidase |
| Antimetabolite | Flucytosine | Inhibit DNA and RNA synthesis via |
| | | conversion of 5-fluorocytosine to 5- |
| | | fluoricil |
| Profens | Flurbiprofen | Direct damage to the fungal cytoplasmic |
| | Ibuprofen | membrane |

By way of another example, a combination of a Drug and an antibacterial agent in a present composition can have unexpectedly advantageous therapeutic control in furuncles. Such a treatment may be especially useful in the earlier stages of boil development (pre-boil eruption). Additional, adequate treatment of pain by the NSAID in the composition can lessen the likelihood that the affected subject will worsen the condition by physical manipulation (e.g. scratching, pinching, etc.)

Pseudomonas folliculitis is another skin condition that is treated by present compositions comprising a Drug and an antibacterial agent. In addition to advantageous therapeutic control, such compositions can be used to prevent hyperpigmentation often characteristic of disease progression.

Impetigo can surprisingly be treated with Drug/antibacterial compositions taught herein. Lesions most often develop in areas where there is a break in the skin, often from physical trauma (e.g. abrasions, scratches, etc). In cases where an inflammatory response to physical trauma weakens the skins defense to bacteria, present combinations of a Drug and antibacterial can have surprising results. Optionally, an antihistamine can be added to the formulation.

Present compositions containing a Drug in combination with an antihistamine can be a surprisingly beneficial treatment for dermatitis (e.g. atopic dermatitis). Optionally, such compositions further comprise a moisturizer or humectant or both.

Present compositions containing a Drug in combination with an antifungal and optionally a keratolytic can be surprisingly beneficial treatment for dermatitis for example seborrheic dermatitis and pityriasis captis. For example, a composition comprising a high dose of Drug (optionally an NSAID, where the NSAID is optionally a macrolid such as-tacrolimus or pimecrolimus can be a surprisingly beneficial treatment for dermatitis.

Combinations of an NSAID and a macrolid in present compositions can have a co-action that allows a superior therapeutic profile despite a macrolid concentration in the composition below its EC50 in a single-Drug composition.

Present compositions containing a Drug in combination with an antihistamine (e.g. H1 or H2 or both) can be a surprisingly beneficial treatment for urticaria (e.g. hives).

Present compositions containing a Drug in combination with one or more of a retinoid, salicylic acid, azelaic acid, benzoyl peroxide, or a topical antibiotic can be a surprisingly beneficial treatment for acne, wounds (e.g. to aid healing), and rosacea. For rosacea, addition of UVA and UVB blockers can be useful.

Present compositions containing a Drug in combination with either or both of (1) a UVA and/or UVB blocker; and (2) an antioxidant can be a surprisingly beneficial treatment for sunburn and actinic keratoses. Moreover, the co-action of such combinations unexpectedly demonstrates enhanced reduction of risk of skin cancer. Without being bound by theory, the applicants believe that acute sun exposure, resulting in erythema and burn, induces an acute inflammatory reaction that plays a role in increased epidermal keratinocyte proliferation and decreased apoptosis contributing to the UV-induced malignant transformation of these cells. Moreover, free radical formation is also involved in the pathogenesis of skin cancer and is blocked by antioxidants. Accordingly, the action of UV blockers and or antioxidants can prevent such proliferation or free radical formation to a level that can be adequately controlled by the anti-inflammatory action of a Drug in present compositions.

Present compositions containing corticosteroids and a Drug at a corticosteroid-sparing concentration are useful for treating severe forms of local inflammatory disease. While corticosteroids can be effective for some skin disorders (e.g. psoriasis), corticosteroids have many well characterized side effects (e.g. Cushing's syndrome, skin thinning, and increased susceptibility to infection). It has been surprisingly discovered that by combining a Drug and corticosteroid, a similarly effective composition can be made while substantially reducing steroid concentration to a safer level (e.g. less than about 50% or less than about 20% of the level that might otherwise be required as a single-Drug composition).

Present compositions containing cyclosporine and a Drug at a cyclosporine-sparing concentration are useful for treating severe forms of local inflammatory disease. While cyclosporine can be effective for some skin disorders (e.g. psoriasis), cyclosporine has many well characterized side effects (e.g., it is carcinogenic). It has been surprisingly discovered that by combining a Drug and cyclosporine, a similarly effective composition can be made while substantially reducing cyclosporine concentration to a safer level (e.g. about 50% of the level that would otherwise be required).

Present compositions containing a Drug in combination with an anti-leishmaniasis agent can be a surprisingly beneficial treatment for leishmaniasis, sunburn, and actinic keratoses. Examples of useful anti leishmaniasis agent are amphotericin, miltefosine, sodium stibogluconate, or a combination of antimony and meglumine antimoniate. Without being bound by theory, the inventors believe a host exhibits a prostaglandin-mediated response to a bite from an insect such as a sandfly and that such response positively affects the infectious cycle. A co-action between a anti-leishmaniasis agent and a Drug in present compositions results in a favorable therapeutica profile.

Table 8 sets forth examples of present compositions useful for treating various local inflammatory conditions.

TABLE 8
Compositions Useful for treating Local inflammatory conditions.

Present Composition,
additional features

| | | |
|---|---|---|
| azelaic acid, benzoyl | Rosacea | |
| peroxide, salicylic acid | Acne | |
| anti-bacterial | Furuncles | Pseudomonas |
| | Impetigo | Folliculitis |
| | Wounds/Infections | Rosacea |
| | | Eczema |
| anti-fungal agent | Dermatophytoses | Pityriasis Capitis |
| | Seborrheic Dermatitis | |
| anti-fungal agent and/or | Seborrheic Dermatitis | Seborrheic |
| keratolytic | Rosacea | Dermatitis |
| | Pityriasis Captis | Pityriasis Capitis |
| anti-histamine | Urticaria | Wounds |
| | Dermatitis | Eczema |
| Antipsoriatic agents | Psoriasis | |
| | Burns | |
| Botanical agent - as the | PFB | Wounds/Infections |
| Drug or as a second Drug | Dermatitis | |
| Clindamycin | Acne | |
| Corticosteroids | Acne | Osteoarthritis, |
| | Eczema | Rhematoid Arthritis, |
| | PFB | And Related Paid. |
| | Seborrheic Dermatitis; | Psoriasis |
| | Pityriasis Capitis | Vitiligo |
| | Allergic And Pruritic | Urticaria |
| | Skin Disorders | Dermatitis |
| Cyclosporine (low | Psoriasis | Severe Dermatitis |
| concentration) | PFB | |
| Fluriprofen or Tumeric oil | Dermatophytoses | |
| (as the Drug or combined | PFB | |
| with the Drug) | | |
| macrolid | Contact Dermatitis; | Pityriasis Capitis |
| | Seborrheic Dermatitis | |
| UVA and/or UVB blocker | PFB | Dermatitis |
| | Sunburn | Rosacea |
| | Actinic Keratoses | Vitiligo (e.g. UVB |
| | Wounds/Infections | blocker) |
| | | PFB |
| UVA and/or UVB blocker | Rosacea | |
| antihistamine | | |

### Local Inflammatory Disorders

The present invention is useful for treating a subject with a local inflammatory disorder, e.g. a disorder in one or more of skin, joints, muscle, and ligaments. Examples of inflammatory skin disorders that can be effectively treated according to the present invention are disorders of the epidermis and dermis.

Non-limiting examples of such a disorders include the eczemas (i.e. eczema and related conditions), psoriasis and related conditions, insect bites, erythrodermas, mycosis fungoides and related conditions, pyoderma gangrenosum, erythema multiforme, rosacea, onychomycosis, acne, boils, and related conditions, UV damage, psoriasis, folliculitis and related conditions such as in-grown toe and finger nails, acne keloidalis, and boils.

Non-limiting examples of eczemas useful for treatment according to the present invention are atopic eczema, Acrodermatitis continua, contact allergic dermatitis, contact irritant dermatitis, atopic dermatitis, dyshydrotic eczema or pompholyx, Lichen simplex chronicus, Nummular eczema, seborrheic dermatitis, and stasis eczema.

Non-limiting examples of folliculitis useful for treatment according to the present invention are Pseudomonas folliculitis (hot tub folliculitis), barber's itch, Tinea barbae, pseudofolliculitis barbae, pityrosporum folliculitis, and herpetic folliculitis.

As used herein, pseudofolliculitis barbae includes pseudofolliculitis of areas other that the beard (barbae). Accordingly, PFB signifies a condition of the skin (or area of the skin) wherein inflammation results from physical trauma caused, at least in part, from hair growth. Accordingly, PFB can affect men with curly hair who shave their faces, women with hirsutism who shave or wax their face, subjects with curly or sharp-tipped hair that shave their legs, arm pits, and the so-called bikini areas (i.e., pubic region, upper thighs, etc) as well as individual who develop hair induced skin inflammation even in the absence of shaving (e.g. ingrown hairs).

PFB subjects that can also be treated with compositions of the present invention in combination with other treatments or activities such as shaving, laser treatment, waxing (for hair removal), or depilatory treatment.

### Local Pain

The present invention is useful for treating a subject with local pain, for example pain resulting from stimulation of nociceptors in the skin, bones, joints, and muscles. One skilled in the art will readily recognize that many or most of the aforementioned local inflammatory disorders further comprise a pain component resulting from stimulation of nociceptors in the skin. Non-limiting examples of such pain that result from stimulation of nociceptors in bones, joints, and muscles usefully treated by compositions of the present invention are arthritis, muscle damage, surgery of bones, joints, and muscles, fibromyalgia, neuropathy, and muscle-cramps. Optionally, embodiments of the present invention also reduce the inflammatory response associated with arthritis.

### Treatment Methods

The present invention provides a method of treating a local inflammatory disorder comprising applying to the skin of a subject in need thereof a composition containing a high concentration of Drug solubilized in a solvent system wherein such application results in local delivery of therapeutic levels of a Drug yet with only low levels of systemic delivery.

Previously described alcoholic gel formulations have some desirable properties, for example U.S. Pat. No. 5,093,133, Example 1 (also set forth herein below in Example 2). It has surprisingly been discovered herein that application of a present composition to the skin in an area affected by (or adjacent to) a local inflammatory disorder can result in therapeutic control comparable or superior to the above cited alcoholic gel of U.S. Pat. No. 5,093,133. By comparable it is meant about 50% to about 100%. By superior it is meant more than about 100% to about 150% or to about 200% or more. Such therapeutic control is especially useful to individuals with an alkanol sensitivity or with a condition where alkanol causes drying deleterious to the skin.

It has surprisingly been discovered herein that application of present compositions to the skin in areas affected by (or adjacent to) a local inflammatory disorder can result in therapeutic control yet with low levels of systemic delivery comparable or superior to the above cited alcoholic gel of U.S. Pat. No. 5,093,133. By comparable it is meant about 100% to about 150% or to about 200%. By superior it is mean about 50% to about 100%. A “low level” of systemic delivery is delivery that results in a circulating Drug level that is less than about 25% of the circulating level that results after the same amount of Drug is administered orally. Optionally, low levels are less than about: 20% or 10% or 5% or 1% of the circulating level that results from the same amount of Drug administered orally. Levels can be expressed as Cₘₐₓ or AUC (0-∞) levels. Systemic delivery can be demonstrated in humans or an animal model such as the minipig.

In one embodiment, therapeutic levels of Drug can be achieved through application of present compositions to the skin at regular intervals such as one or more times per week or one or two or more times per day.

### EXAMPLES

The dermatologically acceptable compositions of the present invention are made in a conventional manner as exemplified herein. Moreover, one skilled in the art can readily understand that the scope of the invention includes other compositions that follow the teaching herein.

The compositions of the present invention are used for the topical delivery of topically active drug to the skin of a human or animal patient in need of such treatment. Specifically, a safe and effective amount of a Drug is applied to the skin in a composition at the site where treatment is required. In specific embodiments, the compositions of the present invention can be used to provide an analgesic or anti-inflammatory effect to the patient by applying a safe and effective amount (e.g., from about 0.002 to about 0.01 g/cm² or to about 0.1 or 1 gm/cm²) of a composition of the present.

The following examples are intended to exemplify the compositions of the present invention, as well as their manufacture and their use. The examples are not intended to be limiting of the scope of the present invention.

### Example 1

### Mouse Ear Edema Assay

Parameters of therapeutic efficacy of present compositions are examined in the mouse ear edema assy. Edema is induced in a mouse ear by topical application of, for example, arachidonic acid (AA), croton oil (CO), or Phorbol-12-Myristate-13-Acetate (TPA). Typically these irritants are administered topically to the inner and/or outer ear in a solvent such as acetone. The amount of irritant varies depending on the selection thereof. For example a typical application of about: 2 mg/ear of AA, 200 μg/ear of CO, or 5 μg/ear of TPA. A composition of the present invention is typically applied before application of an irritant (e.g. one or more days before or one or more hours before) depending upon the parameter being examined. For example, if efficacy of steady state Drug levels is being examined, one or more pre-irritant applications are performed. If penetration pharmaco-kinetics are being examined, pre-irritant applications may be performed at a short interval before irritant challenge (e.g. 30 minutes prior to challenge)

For example, when testing active agents against induced edema, the composition containing an active agent can be administered topically: 30 minutes prior to, or concurrently with the application of AA; concurrently with the application of croton oil; or 30 minutes after the application of TPA. The ear that is not treated with active agent is left as a control, only having the irritant applied to it.

The effect that the composition containing an active agent has on the induced edema is be measured up to 6 hours after the application of irritant in various ways. For example, the change in the ears thickness is measured with a precision micrometer prior to, and after the applications of irritant. This change is divided by the change in thickness of the control ear and then multiplied by 100. This calculation gives the anti-inflammatory effect of the composition as a percent of reduced thickness.

Alternatively the mice are killed (e.g. by cervical dislocation), for example one hour after the irritant is administrated, and the ear is perforated with a metal punch (a 6-mm diameter disc). Edema is assessed by subtracting the weight of the disc from the weight of a control ear. This difference is divided by the weight of the control ear and then multiplied by 100. This calculation gives the anti-inflammatory effect of the composition.

The composition containing the active agent can be administered in different amounts to determine the ED50, the dose of a drug that is pharmacologically effective to achieve 50% of the maximal inhibition.

Efficacy in the mouse ear assay is determined in comparison with an antiinflamatory agent of known activity, for example indomethacin. Indomethacin is especially active in mice in this model and hence can be considered a positive control (or 100%).

### Example 2

### Mini Pig Studies for Toxicokinetics

Compositions of the present invention are characterized for toxicokinetics using in Hanford miniature swine (“minipigs”) in, for example, using a 3-Month study.

Typically, about one gram of a composition is applied daily to a 10 cm×20 cm area on the back of minipigs in a thin layer for a period of days or weeks, for example for 13 weeks.

Systemic bioavailability is examined at various time points each day and throughout the study. Tₘₐₓ and Cₘₐₓ are determined, typically throughout the study.

Evaluations for Drug-related effects are based on clinical observations, weekly dermal irritation scoring, body weights, food consumption, ophthalmology, hematology, coagulation, blood chemistry, urinalysis, organ weight measurements and gross and histopathology. Dermal treatment sites are taken for microscopic examination.

### Example 3

### Pharmacokinetic Analysis by Comparison to Oral Drug Administration

Generally, “C-Drug is administered orally to rats as a single dose and BID doses of 20 mg/kg, and to dogs as a single dose and BID doses of 8 mg/kg. Radioactivity is determined in the plasma after single doses, and the tissue distribution of radioactivity is determined after multiple days of BID dosing. In rats, after single doses, maximal concentration is generally attained in less than an hour (e.g. at approximately 20 minutes), and is followed by a rapid decline to a very low level by, for example, 6 hours after dosing. Most of the plasma content of ¹⁴C is present as unchanged Drug, with metabolites seen. In dogs, after single doses, maximal concentration is obtained at approximately 90 minutes, and is followed by a much slower decline than seen in rats. All of the plasma content of ¹⁴C is present as unchanged Drug. In the rat, after repeated BID dosing of 20 mg/kg, radioactivity accumulates in the adrenals, ovaries, fat, thyroid and skin. Radioactivity is greatest after 1 month of dosing than after 1 week of dosing, demonstrating accumulation in lipophilic tissues. However, in dogs that receive 8 mg/kg BID for up to 14 days, radioactivity does not appear to accumulate, but rather is rapidly excreted in the bile.

### Example 4

### Efficacy in PFB

PFB Efficacy is examined by up to 20 week double blind, placebo controlled, cross-over clinical trial. The Investigator performs a quantitative assessment of PFB lesions at the Baseline at weekly thereafter. Papules, pustules, and ingrown hairs as defined below are counted and recorded.

The primary objectives of this study are:
To determine the efficacy of various NSAID compositions applied at various intervals ranging from every other day to twice per day for five weeks in reducing the signs and symptoms of PFB; and
To determine the safety and tolerability of the various NSAID compositions.

Papules, pustules, and ingrown hairs as defined below are counted and recorded.

Papule: A small solid elevation less than 1.0 cm in diameter

Pustule: A small, circumscribed elevation of the skin which contains yellowwhite exudates

Ingrown Hair: A hair that has exited the skin, curved around and reentered the skin, or a hair that has pierced the follicle and is growing under or in the skin

Lesions are to be counted on the neck, lower left and right cheeks, and jaw line (beard area). The same qualified physician completes the assessment at each visit. Each assessment is performed independent of previous assessments. Subjects have a total of at least 10 (for moderate) of 2 (for mild) follicular papules, pustules, or ingrown hairs at the Baseline Visit to be admitted to the study.

Inflammatory and/or nodulocystic lesions, erythema, and hyperpigmentation are assessed according to the following 6-point Liked (categorical) scale:
0 None: No evidence of active disease
1 Minimal: Rare non-inflammatory lesions present (lesions must be resolving and may be hyperpigmented, though not pink/red). Barely perceptible elevation (discernable by touch only).
2 Mild: Non-inflammatory lesions predominate, with few inflammatory papules/pustules. Light red color. Visible but mild elevation. No nodulocystic lesions.
3 Moderate: Some non-inflammatory lesions are present with multiple inflammatory lesions evident. Definite lesion redness and elevation. There may or may not be one small nodulocystic lesion.
4 Severe: Highly inflammatory lesions predominate. Deep intense red color. Marked dermal swelling and induration in widespread areas. There may or may not be a few nodulocystic lesions.
5 Very Severe: Many nodulocystic lesions. Results are recorded in the source document and on the appropriate CRF. The same qualified physician will complete the assessment at each visit. Each assessment should be performed independent of previous assessments. Subjects must have a rating of at least moderate (3) at the Baseline Visit to be admitted to the study.

All subjects are asked to evaluate specific PFB symptoms of itch, pain, and shaving discomfort, as well as the overall condition of their PFB at the Baseline, and weekly thereafter (“Subject's Assessment of Symptoms”)

Subjects complete the following 5-point Likert (categorical) scale for each symptom and for overall condition:
0 None: symptom/overall PFB condition absent
1 Mild: symptom/overall PFB condition present but not particularly bothersome
2 Moderate: symptom/overall PFB condition present and bothersome, but does not interfere with daily activities
3 Severe: symptom/overall PFB condition present and bothersome and interferes with some daily activities
4 Very Severe: symptom/overall PFB condition present and bothersome and prevents many normal daily activities. Each assessment is performed independent of previous assessments.

Global Assessment of Improvement. Subjects are asked to compare the overall condition of their PFB at the Week 2, 4, and 6. Visits with the overall condition before treatment using the following 5-point Likert (categorical) scale:
2 Overall condition and shaving comfort much better than before Treatment
1 Overall condition and shaving comfort slightly better than before Treatment
0 Overall condition and shaving comfort unchanged, same as before Treatment
−1 Overall condition and shaving comfort slightly worse than before Treatment
2 Overall condition and shaving comfort much worse than before Treatment

Each assessment is performed independent of previous assessments.

### Example 5

### Adverse Events

Compositions are analyzed for common adverse events typical of orally administered NSAIDs, for example, for events related to the cardiovascular system (e.g. edema, fluid retention), the digestive system (e.g. nausea, epigastric pain, heartburn, diarrhea, abdominal distress, nausea and vomiting, indigestion, constipation, abdominal cramps or pain, fullness of GI tract), the nervous system (dizziness, headache, nervousness), skin and appendages (e.g. rash, including maculopapular type), and pruritus) and the specal senses (e.g. tinnitus).

### Example 6

### In Vitro Percutaneous Penetration

Absorption and penetration of present compositions are studied using excised human skin from elective surgery procedures described in the FDA and AAPS Report of the Workshop on Principles and Practices of In Vitro Percutaneous Penetration Studies: Relevance to Bioavailability and Bioequivalence (Pharm. Res. 4:265, 87).

All compositions are spiked with tracer levels (˜1.0 μCi/3.2 mg composition dosed per diffusion cell) of [³H]-ibuprofen. A single clinically relevant, finite, dose (˜5 mg composition/cmP ²P) is applied to dermatomed human abdominal skin from elective surgery. Percutaneous absorption is evaluated using this skin mounted on Bronaugh flow-through diffusion cells maintained at a constant temperature of 32° C. by use of recirculating water baths. These cells have an opening with a nominal area of 0.64 cmP 2P. Fresh receptor fluid, PBS containing 0.1% sodium azide and 1.5% Oleth 20, is continuously pumped under the dermis at a flow rate of 1 ml/hr and collected in 6-hour intervals. Following a 24-hour duration of composition exposure to the skin, composition residing on the skin surface is removed by wiping with two, dry, cotton swabs. To remove any residual composition remaining on the skin surface, the upper layers of the stratum corneum are removed from the epidermis with a single cellophane tape-strip. The remaining epidermis is then physically separated from the dermis and processed for analysis separately. Quantity of radioactivity in the wipes, tape-strip, epidermis, dermis, and receptor fluid samples is determined using liquid scintillation counting techniques.

### Example 7

### Microdialysis to Determine Pharmacokinetics of Topically Applied Instant Compositions

A microdialysis probe is implanted into the tissue of interest and is constantly perfused with a saline at a low flow rate of 1 to 10 μL/min. Substances in the interstitial space fluid pass the membrane by passive diffusion along their concentration gradient resulting in a certain concentration in the perfusion medium. This dialysate is collected at timed intervals and is subjected ex vivo to different types of chemical analyses, which can be performed either in an off-line or on-line fashion. Depending on the molecular cut-off of the membrane, large molecules such as proteins are usually excluded from the dialysate, which enables analysis without time-consuming sample preparation or sample storage without the immediate fear of enzymatic degradation. Often microdialysis is performed under nonequilibrium conditions, and dialysate concentrations represent only a fraction of actual concentrations in the medium surrounding the microdialysis probe. To obtain and quantify interstitial space fluid concentrations from dialysate concentrations, microdialysis probes are calibrated. Microdialysis provides selective access to the unbound and thus pharmacologically active drug fraction in the interstitial space fluid of tissues.

Microdialysis can be used, for example, to determine the following:
√(Kd/Dd) by regressing concentration vs depth of probe
steady-state epidermis-dermis interface concentration (here a thickness of 30 μm and dermis of tape-stripped rats is assumed) and use this to calculate De/√(Dd*Kd)
De from non-steady state data of tape-stripped rats were by Dd and Kd are found
Dc from non-steady state data on intact skin using all previous estimates

### Example 8

### Alkanolic Comparator Composition

An alcoholic (alkanolic) composition (e.g. 54%, alkanol) of U.S. Pat. No. 5,093,133 (Example 1), set forth in Table 9 is made and used as a comparator to compositions of the present invention.

TABLE 9
Hydroalcoholic Gel as a Comparator

| | |
|---|---|
| S-ibuprofen (SEPRACOR, INC.) | 10 |
| (Substantially pure, about 97% S-ibuprofen) | |
| Alcohol USP | 54 |
| Propylene Glycol USP (PG) | 5 |
| Purified Water USP | 28.25 |
| Methylparaben NF | 0.1 |
| Propylparaben NF | 0.1 |
| Triethanolamine | 0.25 |
| Hydroxypropyl Cellulose NF (HPC) | 2.5 |
| (KLUCEL HF) | |
| (Apparent viscosity 1500-2500 cps) | |

### Example 9

### Prodrug Analysis

It has been discovered that, in one embodiment, compositions of the present invention, upon storage, result in the generation of a prodrug form of the Drug.

HPLC analysis was performed on composition of 15% ibuprofen, about 60% ethanol, 3% glycerin, and 2% propylene glycol stored for 3 months at 25° C. A new peak (i.e., the prodrug) distinct from the ibuprofen peak was detected within the chromatographic profile. The peak showed an elution position considerably later than Ibuprofen and a UV response at 220 nm.

Next, the peak was characterized in terms of retention position, UV spectrum and mass spectroscopy response. In addition, isolates of the peak were collected from the chromatograph system employed for liquid chromatography-mass spectroscopy.

Next, two grams of composition 1a were diluted in twenty-five milliliters of (50:50) water:acetonitrile. The solution was centrifuged and the supernatant collected for analysis.

Chromatography was conducted as follows:
Pumps: Hewlett Packard Model 1100 Binary Systems
Solvent A: Water
Solvent B: Acetonitrile
Gradient: Start 40% B
   Raise to 60% B at 20 minutes
   Raise to 90% B at 40 minutes
Flow Rate: 1.0 ml/min
Stationary Phase Zorbax CS (4.6×150 mm)
Column Temperature: 25 C
Injection volume 25 L

Sequential detection was performed by UV absorbance using an HP diode array detector followed by ESI-MS followed by ESI-MS using a Sciex Q-Star/Pulsar quadrupole-TOF mass spectrometer operating in either the positive and negative ion modes.

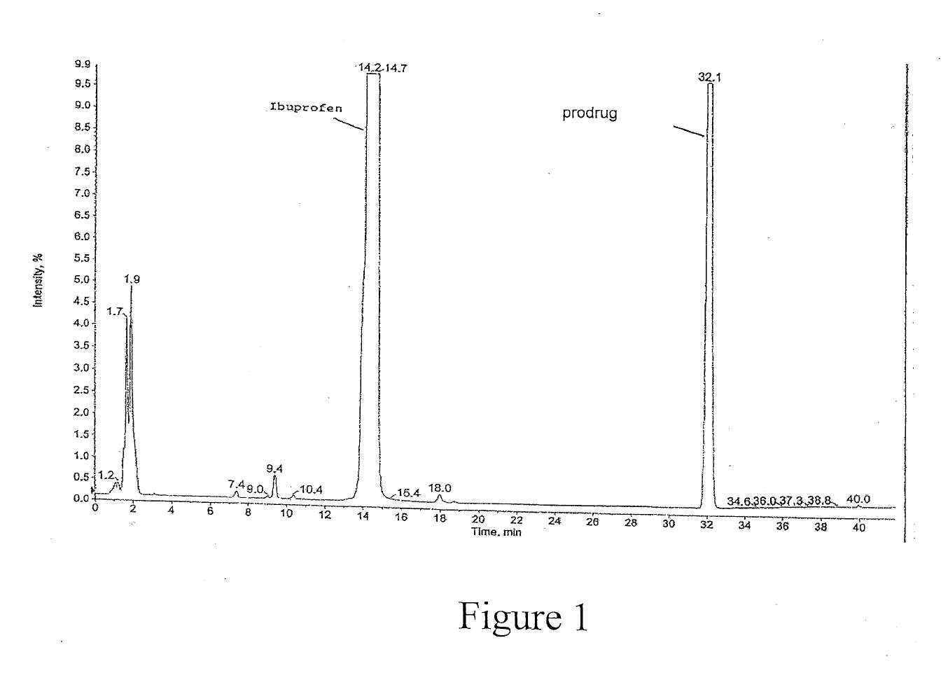

FIG. 1 illustrates the UV chromatogram (220 nm) following injection of Composition 1a stored 3 months at 25° C. using the chromatographic conditions described above. Ibuprofen showed a peak at about 14 minutes and the prodrug showed a peak at about 32 minutes.

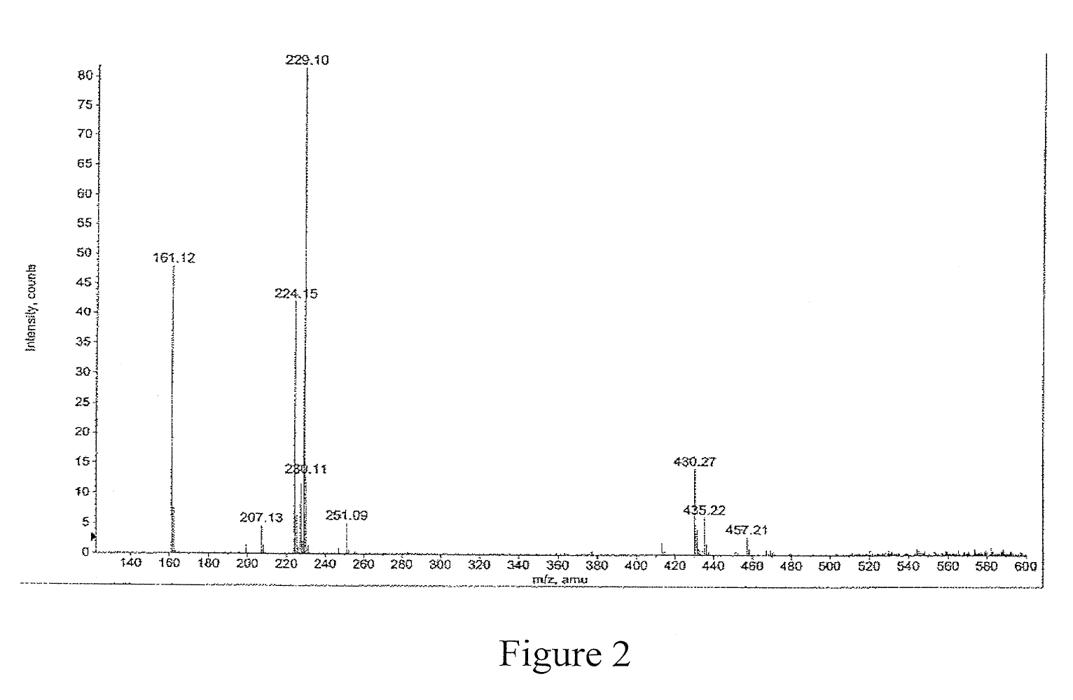

FIG. 2 shows the positive ESI mass spectrum for the Ibuprofen peak. The expected (M+H)+ pseudomolecular ion is observed at m/z 207.13 with corresponding (M+NH₄)⁺ and (M+Na)⁺ pseudomolecular ions at m/z 224.15 and 229.10 respectively. Dimeric cluster ions may be assigned to signals at m/z 430.27 and m/z 435.22. A notable, possible fragment ion also appears at m/z 161.12 consistent with decarboxylation as illustrated below:

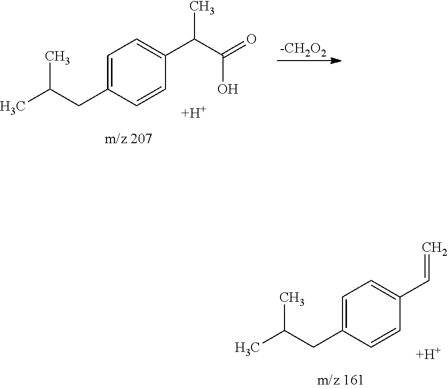

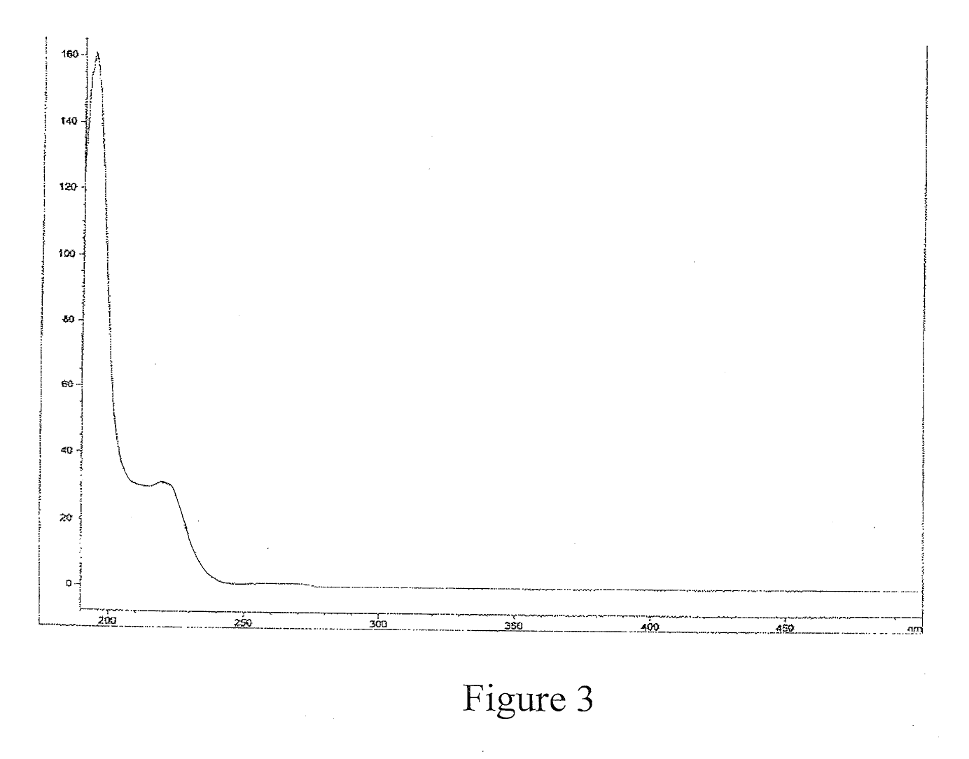

FIG. 3 shows the UV spectrum for the Ibuprofen which demonstrates maxima at approximately 220 nm and 265nm.

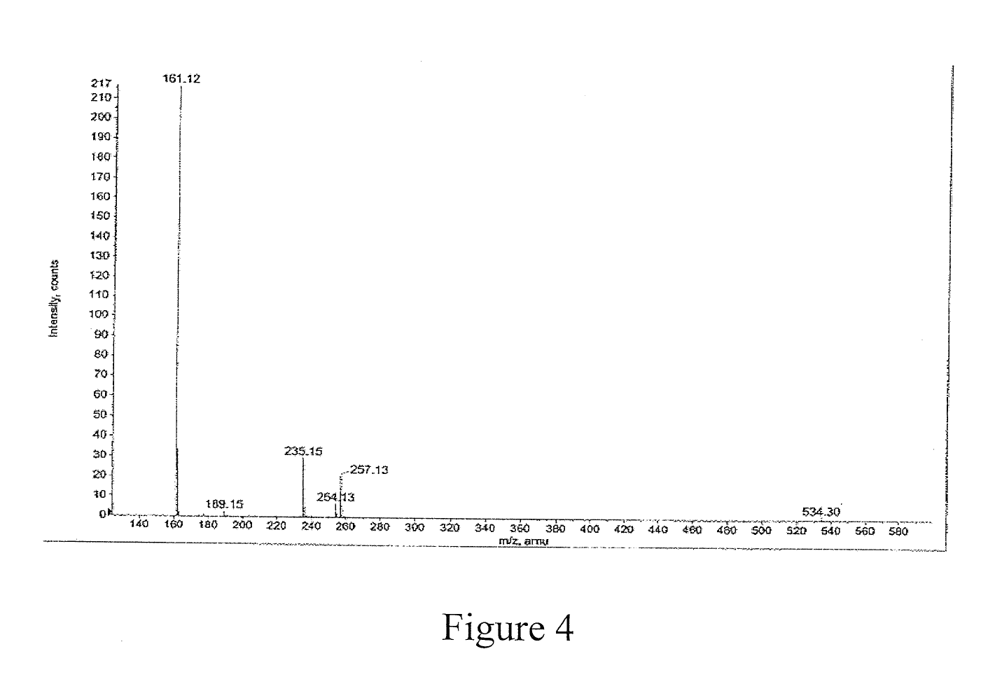

FIG. 4 shows the positive ESI mass spectrum obtained from the prodrug. A possible (M+H)⁺ is observed at m/z 235.15 and, as in the Ibuprofen data, corresponding (M+NH₄)⁺ and (M+Na)⁺pseudomolecular ions may be assigned at m/z 254.13 and m/z 257.13 respectively. Of note is the signal at m/z 161.12 consistent with the same fragment ion described for Ibuprofen.

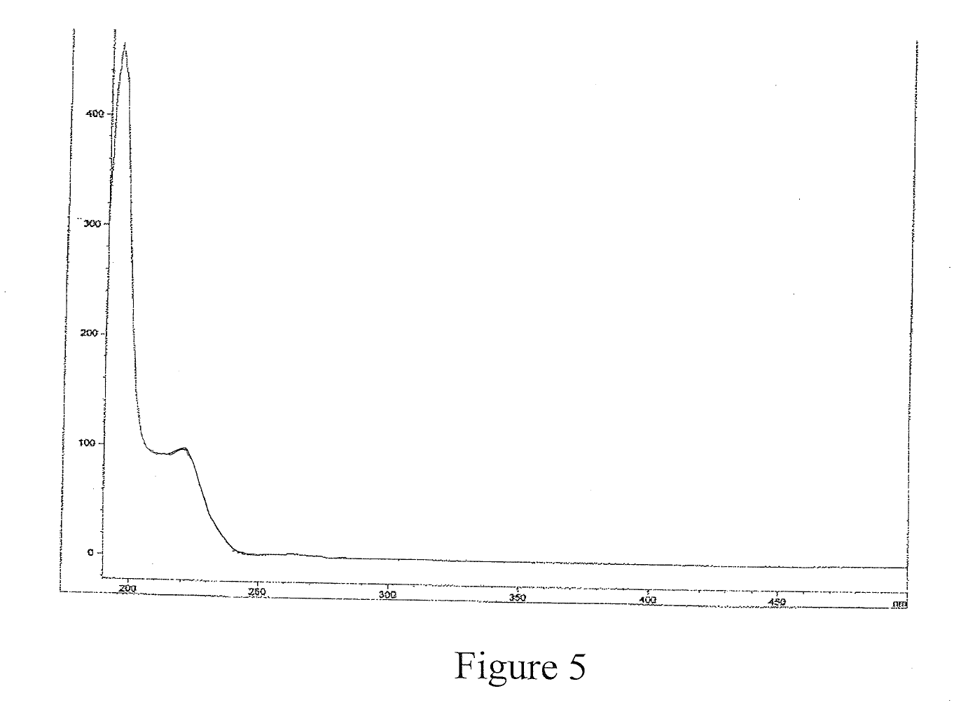

FIG. 5 shows the UV spectrum obtained from the prodrug and is very similar to that obtained for Ibuprofen with maxima at approximately 220 nm and 265 nm.

The data obtained during this study indicate that the prodrug has (1) a neutral mass of 234.15 Da; (2) a UV spectrum very similar to that of Ibuprofen; (3) retention behavior that suggests it to be considerably more hydrophobic than ibuprofen; (4) no significant negative ion MS response; and (5) a positive ion MS spectrum indicating a shared fragment with ibuprofen.

These data support the identity of the prodrug being ethylisobutylphenylpropionate.

### Example 10

### Effect of pH on Prodrug Formation Rate

The effect of two different pHs on the generation of prodrug was examined in a composition comprising 15% ibuprofen, about 60% ethanol, 3% glycerin, and 2% propylene glycol.

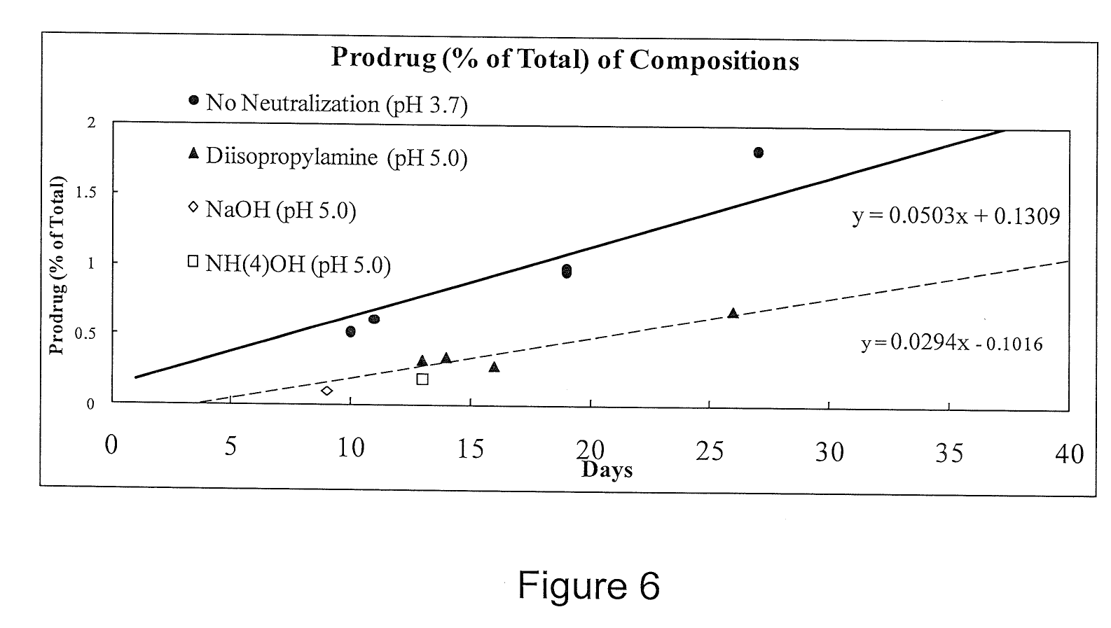

As shown in FIG. 6, prodrug generation is linear for at least the first 26 days. At pH 3.7, that rate was approximately 0.05% per day as compared to the lower rate of about 0.025% per day at pH 5.

### Example 11

### Drug Concentration, pH and Prodrug Generation

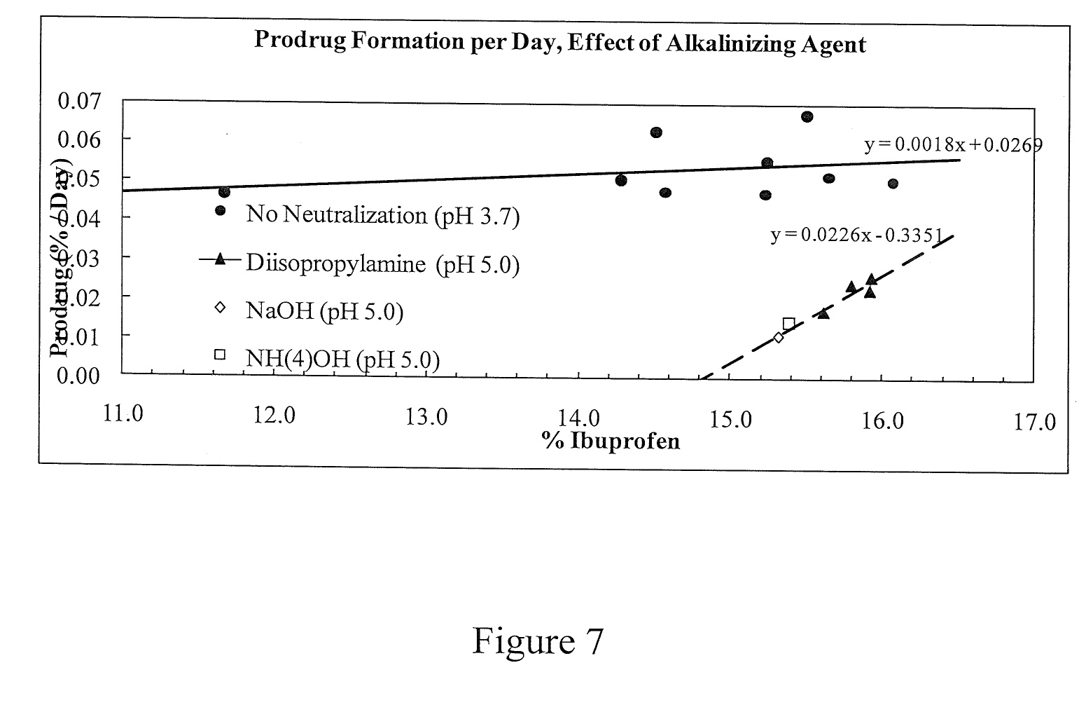

The effect of Drug concentration and pH on prodrug generation was examined, Compositions with the indicated amounts of ibuprofen were made using ethanol as a solvent. As can be seen in FIG. 7, higher pH (e.g. 5.0) substantially decreases the rate of prodrug formation. At acidic pH (e.g. 3.7), prodrug formation was relatively independent of Drug concentration. However, at pH 5, prodrug formation seemed to be first order (i.e. dependant upon the Drug concentration).

In present compositions, decreasing the concentration of active drug in pH 5 compositions substantially decreases the rate of prodrug formation. At high Drug concentration, (e.g. above about: 12% or 15%), present compositions with low alkanol content have low rate of prodrug formation.

### Example 12

### Compositions

The composition set forth in Table 10 was made as follows:

TABLE 10

| Component | w/w % |
|---|---|
| Drug | 15 |
| PEG 400 | 20 |
| Propylene Glycol | 10 |
| Benzyl Alcohol | 3 |
| Potassium | 3.5 |
| Carbonate | |
| Salicylic Acid | 0.15 |
| Nanopure Water | 47.35 |
| Hydroxypropyl | 1 |
| cellulose (Klucel | |
| HXF) | |
| Total | 100 |

a) Potassium carbonate was dissolved in water;
b) PEG 400, propylene glycol and salicylic acid were added and mixed with the potassium carbonate/water mixture;
c) Drug was added and stirred until dissolved;
d) Mixture was heated to 35° C. for 20 minutes;
e) Benzyl alcohol was added;
f) Klucel was dispersed in water at 55° C. and then added to the mixture;
g) Additional water was added; mixture stirred until homogeneous.

The pH was 6.815 and the composition was a single phase.

### Example 13

### Compositions

The composition set forth in Table 11 was made as follows:

TABLE 11

| Component | w/w % |
|---|---|
| Drug | 15 |
| Ethanol | 30 |
| PEG 300 | 20 |
| D-panthenol | 0.15 |
| Propylene glycol | 2 |
| Salicylic acid | 0.15 |
| Distilled water | 32 |
| Carbopol Ultrez 10 | 0.25 |
| Total | 100 |

The composition was made in the following manner:
Dissolve all alcohol soluble ingredients in ethanol and PEG 300;
Add optional liquid components;
Add water; heat mixture to 45° C. for 60 minutes;
Add the thickening agent slowly with agitation an allow thickening agent to hydrate for 18 hours; mixture stirred until homogeneous

The pH was 2.91 and the composition was single phase.

### Example 14

### Compositions

TABLE 12

| Component | w/w % |
|---|---|
| Ibuprofen (Na salt) | 15 |
| PEG 300 | 20 |
| D-panthenol | 0.15 |
| Propylene glycol | 10 |
| Salicylic acid | 0.15 |
| Polysorbate 20 | 2 |
| Distilled water | 52 |
| Hydroxypropyl | 0.6 |
| cellulose (Klucel HF) | |
| Total | 100 |

The composition of Table 12 was made in the following manner:
Dissolve all dry ingredients in PEG 300, propylene glycol and water;
Add optional liquid components;
Adjust pH with HCl to 6.95
Klucel was dispersed in water at 55° C. and then added to the mixture;
mixture stirred until homogeneous

The pH was 6.7, the viscosity was 779 cps, and the composition was single phase.

### Example 15

### Compositions

TABLE 13

| Component | w/w % |
|---|---|
| Ibuprofen (Na salt) | 15 |
| Ethanol | 20 |
| D-panthenol | 0.15 |
| Propylene glycol | 10 |
| Salicylic acid | 0.15 |
| Polysorbate 20 | 2 |
| Glycerin | 3 |
| Distilled water | 49 |
| Hydroxypropyl | 0.5 |
| cellulose (Klucel HF) | |
| Total | 100 |

The composition Table 13 was made in the following manner:
Dissolve all dry ingredients in ethanol, propylene glycol and water;
Add optional liquid components;
Adjust pH with HCl to 6.59
Klucel was dispersed in water at 55° C. and then added to the mixture;
   mixture stirred until homogeneous

The pH was 6.58, the viscosity was 194 cps, and the composition was single phase.

### Example 16

### Compositions

TABLE 14

| Component | w/w % |
|---|---|
| Drug (salt) | 15 |
| Isopropanol | 20 |
| D-panthenol | 0.15 |
| Propylene glycol | 10 |
| Salicylic acid | 0.15 |
| Polysorbate 20 | 2 |
| Glycerin | 3 |
| Distilled water | 49 |
| Hydroxypropyl | 0.5 |
| cellulose (Klucel HF) | |
| Total | 100 |

The composition of Table 14 was made in the following manner:
Dissolve all dry ingredients in isopropanol, propylene glycol and water;
Add optional liquid components;
Adjust pH with HCl to 6.99
Klucel was dispersed in water at 55° C. and then added to the mixture;
   mixture stirred until homogeneous

The pH was 6.6, the viscosity was 299 cps, and the composition was single phase.

### Example 17

### Compositions

TABLE 15

| Component | w/w % |
|---|---|
| Drug | 15 |
| Ethanol | 20 |
| PEG 400 | 40 |
| D-panthenol | 0.15 |
| Propylene glycol | 3 |
| Salicylic acid | 0.15 |
| Polysorbate 20 | 2 |
| Glycerin | 3 |
| Distilled water | 16 |
| Carbopol Ultrez 10 | 1 |
| Total | 100 |

The composition of Table 15 was made in the following manner:
Dissolve all alcohol soluble ingredients in ethanol and PEG 400;
Add optional liquid components;
Add water;
Add the thickening agent slowly with agitation an allow thickening agent to hydrate for 18 hours; mixture stirred until homogeneous

The pH was 4.43, the viscosity was 239 cps, and the composition was single phase.

### Example 18

### Compositions

TABLE 16

| Component | w/w % |
|---|---|
| Drug | 15 |
| Isopropanol | 25 |
| PEG 400 | 30 |
| D-panthenol | 0.15 |
| Propylene glycol | 3 |
| Salicylic acid | 0.15 |
| Polysorbate 20 | 2 |
| Glycerin | 3 |
| Distilled water | 21 |
| Carbopol Ultrez 10 | 1 |
| Total | 100 |

The composition of Table 16 was made in the following manner:
Dissolve all alcohol soluble ingredients in isopropanol and PEG 400;
Add optional liquid components;
Add water;
Add the thickening agent slowly with agitation an allow thickening agent to hydrate for 18 hours; mixture stirred until homogeneous.

The pH was 4.62, the viscosity was 79 cps, and the composition was single phase.

### Example 19

### Compositions

TABLE 17

| Component | w/w % |
|---|---|
| Drug | 15 |
| Isopropanol | 20 |
| PEG 400 | 30 |
| D-panthenol | 0.15 |
| Propylene glycol | 2.5 |
| Salicylic acid | 0.15 |
| Polysorbate 20 | 2 |
| Glycerin | 3 |
| Distilled water | 26 |
| Carbopol Ultrez 10 | 1 |
| Total | 100 |

The composition of Table 17 was made in the following manner:
Dissolve all alcohol soluble ingredients in isopropanol and PEG 400;
Add optional liquid components;
Add water;
Add the thickening agent slowly with agitation an allow thickening agent to hydrate for 18 hours; mixture stirred until homogeneous.

The pH was 4.50, the viscosity was 187 cps, and the composition was single phase.

### Example 20

### Compositions

TABLE 18

| Component | w/w % |
|---|---|
| Drug | 14 |
| Ethanol | 27 |
| PEG 400 | 27 |
| D-panthenol | 0.15 |
| Propylene glycol | 2 |
| Salicylic acid | 0.15 |
| Polysorbate 20 | 2 |
| Glycerin | 3 |
| Distilled water | 24 |
| Hydroxypropyl | 0.5 |
| cellulose (Klucel HF) | |
| Total | 100 |

The composition of Table 18 was made in the following manner:
Dissolve all alcohol soluble ingredients in ethanol and PEG 400;
Add optional liquid components;
Add water;
Adjust pH with Tris Amino and NaOH to 6.45
Klucel was dispersed in water at 55° C. and then added to the mixture;
mixture stirred until homogeneous

The pH was 6.51, the viscosity was 499 cps, and the composition was single phase.

### Example 21

### Compositions

TABLE 19

| Component | w/w % |
|---|---|
| Drug | 15 |
| Ethanol | 20 |
| PEG 400 | 55 |
| D-panthenol | 0.15 |
| Propylene glycol | 2 |
| Salicylic acid | 0.15 |
| Polysorbate 20 | 2 |
| Glycerin | 3 |
| Distilled water | 2 |
| Carbopol Ultrez 10 | 1 |
| Total | 100 |

The composition of Table 19 was made in the following manner:
Dissolve all alcohol soluble ingredients in ethanol and PEG 400;
Add optional liquid components;
Add water;
Add the thickening agent slowly with agitation an allow thickening agent to hydrate for 18 hours; mixture stirred until homogeneous.

The pH was 4.94, the viscosity was 106 cps, and the composition was single phase.

### Example 22

### Compositions

TABLE 20

| Component | w/w % |
|---|---|
| Drug | 15 |
| Ethanol | 20 |
| PEG 400 | 30 |
| D-panthenol | 0.15 |
| Propylene glycol | 2 |
| Salicylic acid | 0.15 |
| Polysorbate 20 | 2 |
| Glycerin | 3 |
| Distilled water | 27 |
| Carbopol Ultrez 10 | 1 |
| Total | 100 |

The composition of Table 20 was made in the following manner:
Dissolve all alcohol soluble ingredients in ethanol and PEG 400;
Add optional liquid components;
Add water;
Add the thickening agent slowly with agitation an allow thickening agent to hydrate for 18 hours; mixture stirred until homogeneous.

The pH was 4.23, the viscosity was 630 cps, and the composition was biphasic.

### Example 23

### Compositions

TABLE 21

| Component | w/w % |
|---|---|
| Drug | 15 |
| Ethanol | 10 |
| PEG 400 | 65 |
| D-panthenol | 0.15 |
| Propylene glycol | 2 |
| Salicylic acid | 0.15 |
| Polysorbate 20 | 2 |
| Glycerin | 3 |
| Distilled water | 2 |
| Carbopol Ultrez 10 | 1 |
| Total | 100 |

The composition of Table 21 was made in the following manner:
Dissolve all alcohol soluble ingredients in ethanol and PEG 400;
Add optional liquid components;
Add water;
Add the thickening agent slowly with agitation an allow thickening agent to hydrate for 18 hours; mixture stirred until homogeneous.

The pH was 4.95, the viscosity was 359 cps, and the composition was single phase.

### Example 24

### Compositions

TABLE 22

| Component | w/w % |
|---|---|
| Drug | 15 |
| Ethanol | 10 |
| PEG 400 | 50 |
| D-panthenol | 0.15 |
| Propylene glycol | 2 |
| Salicylic acid | 0.15 |
| Polysorbate 20 | 2 |
| Glycerin | 3 |
| Distilled water | 17 |
| Carbopol Ultrez 10 | 1 |
| Total | 100 |

The composition of Table 22 was made in the following manner:
Dissolve all alcohol soluble ingredients in ethanol and PEG 400;
Add optional liquid components;
Add water;
Add the thickening agent slowly with agitation an allow thickening agent to hydrate for 18 hours; mixture stirred until homogeneous.

The pH was 4.49, the viscosity was 239, and the composition was single phase.

### Example 25

### Compositions

TABLE 23

| Component | w/w % |
|---|---|
| Drug | 15 |
| Ethanol | 10 |
| PEG 400 | 40 |
| D-panthenol | 0.15 |
| Propylene glycol | 2 |
| Salicylic acid | 0.15 |
| Polysorbate 20 | 2 |
| Glycerin | 3 |
| Distilled water | 27 |
| Carbopol Ultrez 10 | 1 |
| Total | 100 |

The composition of Table 23 was made in the following manner:
Dissolve all alcohol soluble ingredients in ethanol and PEG 400;
Add optional liquid components;
Add water;
Add the thickening agent slowly with agitation an allow thickening agent to hydrate for 18 hours; mixture stirred until homogeneous.

The pH was 4.42, the viscosity was 139 cps, and the composition was biphasic.

### Example 26

### Compositions

TABLE 24

| Component | w/w % |
|---|---|
| Drug | 15 |
| PEG 400 | 40 |
| D-panthenol | 0.15 |
| Propylene glycol | 10 |
| Salicylic acid | 0.15 |
| Polysorbate 20 | 2 |
| Glycerin | 3 |
| Distilled water | 27 |
| Carbopol Ultrez 10 | 1 |
| Total | 100 |

The composition of Table 24 is made as follow:
Dissolve Drug in PG and PEG 400; warming if necessary;
Dissolve other alcohol soluble or miscible components;
Add other liquid components;
Add water;
Add the thickening agent slowly with agitation an allow thickening agent to hydrate for 18 hours; mixture stirred until homogeneous.

### Example 27

### Solubility of Drugs in Solvent Alcohols

The solubility of examplary Drugs in solvent alcohols of the present invention were determined and are set forth below in Table 25. Briefly, the indicated NSAID was weighed and added to a beaker. To each beaker, incremental amounts of the indicated solvent was weighed and added. The beakers were sealed with aluminum foil and placed at room temperature or in a 35 C water bath for 20-30 minutes. They beakers were shaken frequently to mix the contents. After 20-30 minutes the solvent alcohols were examined. If a single phasic solution was not yet achieved, an additional aliquot of the solvent was added and the cycle was repeated until complete solvation had occurred.

TABLE 25
Maximum Drug Concentrations in single Alcohols

| | | | |
|---|---|---|---|
| EtOH | IPA | PEG | PG |

Solubility at 35 C.

| | | | | |
|---|---|---|---|---|
| Ibuprofen | 50.00 | 47.62 | 25.00 | 20.00 |
| Ketoprofen | 50.00 | 33.33 | 25.00 | 20.00 |
| Diclofenac | 14.29 | | 8.33 | 14.29 |
| Acetaminophen | 16.67 | 8.33 | 10.00 | 10.00 |
| Flurbiprofen | 33.33 | 25.00 | 33.33 | 14.29 |
| Naproxen | 5.88 | 4.17 | 9.09 | 2.78 |

Solubility at Room Termperature

| | | | | |
|---|---|---|---|---|
| Ibuprofen | 37.04 | 35.71 | 22.22 | 15.38 |
| Ketoprofen | 25.00 | 16.67 | 14.29 | 8.33 |
| Diclofenac | 11.11 | | 7.69 | 14.29 |
| Acetaminophen | 9.52 | 4.35 | 5.41 | 4.76 |
| Flurbiprofen | 16.67 | 14.29 | 12.50 | 6.25 |
| Naproxen | 3.70 | 2.63 | 2.78 | 14.29 |
| Niflumic | 3.23 | | | |
| Bufexamac | 1.23 | 0.62 | | |
| Indomethacin | 0.25 | 0.10 | 0.05 | 0.05 |

The results in Table 25, when combined with the teachings herein, allow the skilled artisan to select an NSAID and solvent alcohols useful according to the present invention.

| | | | | Observed | | Predicted | | Super- |
|---|---|---|---|---|---|---|---|---|
| PG | PEG | | | Ketoprofen | | Ketoprofen | | solvent |
| Grams | 400 | EtOH | IPA | | % | | % | Effect |
| (gr) | gr | gr | gr | gr | W/W | gr | W/W | % |
| 4 | | | | 1 | 20.0 | | | |
| | 3 | | | 1 | 25.0 | | | |
| | | 1 | | 1 | 50.0 | | | |
| | | | 2 | 1 | 33.3 | | | |
| 4 | 3 | | | 3.5 | 22.2 | 2 | 22.2 | 50.0 |
| 4 | | 1 | | 2.5 | 28.6 | 2 | 28.6 | 16.7 |
| 4 | | | 2 | 3 | 25.0 | 2 | 25.0 | 33.3 |
| | 3 | 1 | | 2.5 | 33.3 | 2 | 33.3 | 15.4 |
| | 3 | | 2 | 3 | 28.6 | 2 | 28.6 | 31.3 |
| | | 1 | 2 | 2.5 | 40.0 | 2 | 40.0 | 13.6 |
| 4 | 3 | 1 | | 4.5 | 27.3 | 3 | 27.3 | 32.0 |
| 4 | 3 | | 2 | 5 | 25.0 | 3 | 25.0 | 42.9 |
| | 3 | 1 | 2 | 4.5 | 30.0 | 3 | 30.0 | 30.4 |
| 4 | | 1 | 2 | 4.5 | 33.3 | 3 | 33.3 | 28.6 |
| 4 | 3 | 1 | 2 | 5.5 | 28.6 | 4 | 28.6 | 24.2 |

### Example 29

### Super Solvent Effect for Ibuprofen

As shown in Table 28, the solvent systems of the present invention demonstrate remarkable super-solvent effects with ibuprofen.

Table 28 This example illustrates a technical feature of compositions of the present invention; namely that the solvent systems (comprised of at least two solvent alcohols) are able to solubilize a class of NSAIDs to an unexpectedly high level (the “super-solvent” effect). In this example, naproxen is the examplary NSAID. The compositions contain a solvent system comprised of combinations of 2 or more solvent alcohols PEG, PG, ethanol, and isopropanol.

Methods. First, the amount of each solvent alcohol required to solubilize one gram of NSAID was determined (saturation amount). Next, each combination of 2,3 and 4 solvent alcohols were made using the saturation amounts determined by the first step. Finally, the amount of drug that could be solubilized by the solvent systems (i.e. the solvent alcohol combinations) was determined. The super-solvent effect was determined using this protocol, where per cent=actual grams solubilized divided by the predicted grams (based upon the first step) expressed as per cent increase (e.g. if twice the predicted amount could be solubilized, the super-solvent effect would be % 100.

As shown in Table 26, 1 gram (gr) of naproxen can be solubilized by either 35 grams of propylene glycol, 10 grams of PEG 400, 16 grams of ethanol, or 23 grams of isopropanol.

However, when propylene glycol is combined with a polyethylene glycol, the solvent system can solubilize 124% of the amount of NSAID predicted by the combined solvent capacities of the individual solvent alcohols. When PEG is combined with an ethanol or isopropanol, the resultant solvent system can solubilize 166% or 168% of that predicted (respectively) As defined herein, this is shown in Table 26 as a 66% or 68% super-solvent effect. Similarly, when propylene glycol is combined with an alkanol, the super solvent effect is about 24%. When the solvent system comprises 3 or 4 solvent alcohols, there is also dramatic super-solvent effects.

TABLE 26
Super-Solvent Effects with Naproxen

| | | | | | | | | Super- |
|---|---|---|---|---|---|---|---|---|
| PG | PEG | | | Observed | | Predicted | | solvent |
| Grams | 400 | EtOH | IPA | Naproxen | | Naproxen | | Effect |
| (gr) | gr | gr | gr | gr | % W/W | gr | % W/W | % |
| 35.00 | | | | 1.00 | 2.78 | | | |
| | 10.00 | | | 1.00 | 9.09 | | | |
| | | 16.00 | | 1.00 | 5.88 | | | |
| | | | 23.00 | 1.00 | 4.17 | | | |
| 35.00 | 10.00 | | | 2.50 | 5.26 | 2.00 | 4.26 | 23.68 |
| 35.00 | | 16.00 | | 2.50 | 4.67 | 2.00 | 3.77 | 23.83 |
| 35.00 | | | 23.00 | 2.50 | 4.13 | 2.00 | 3.33 | 23.97 |
| | 10.00 | 16.00 | | 3.50 | 11.86 | 2.00 | 7.14 | 66.10 |
| | 10.00 | | 23.00 | 3.50 | 9.59 | 2.00 | 5.71 | 67.81 |
| | | 16.00 | 23.00 | 2.50 | 6.02 | 2.00 | 4.88 | 23.49 |
| 35.00 | 10.00 | 16.00 | | 4.50 | 6.87 | 3.00 | 4.69 | 46.56 |
| 35.00 | 10.00 | | 23.00 | 3.50 | 4.90 | 3.00 | 4.23 | 15.85 |
| 35.00 | | 16.00 | 23.00 | 4.50 | 5.73 | 3.00 | 3.90 | 47.13 |
| | 10.00 | 16.00 | 23.00 | 5.00 | 9.26 | 3.00 | 5.77 | 60.49 |
| 35.00 | 10.00 | 16.00 | 23.00 | 6.50 | 7.18 | 4.00 | 4.55 | 58.01 |

### Example 28

### Super Solvent Effect for Ketoprofen

As shown in Table 27, the solvent systems of the present invention demonstrate remarkable super-solvent effects with Ketoprofen.

TABLE 27
Super-Solvent Effects withy Ketoprofen.

| | | | | | | | | Super- |
|---|---|---|---|---|---|---|---|---|
| PG | PEG | | | Observed | | Predicted | | solvent |
| Grams | 400 | EtOH | IPA | Ketoprofen | | Ketoprofen | | Effect |
| (gr) | gr | gr | gr | gr | % W/W | gr | % W/W | % |
| 4 | | | | 1 | 20.0 | | | |
| | 3 | | | 1 | 25.0 | | | |
| | | 1 | | 1 | 50.0 | | | |
| | | | 2 | 1 | 33.3 | | | |
| 4 | 3 | | | 3.5 | 22.2 | 2 | 22.2 | 50.0 |
| 4 | | 1 | | 2.5 | 28.6 | 2 | 28.6 | 16.7 |
| 4 | | | 2 | 3 | 25.0 | 2 | 25.0 | 33.3 |
| | 3 | 1 | | 2.5 | 33.3 | 2 | 33.3 | 15.4 |
| | 3 | | 2 | 3 | 28.6 | 2 | 28.6 | 31.3 |
| | | 1 | 2 | 2.5 | 40.0 | 2 | 40.0 | 13.6 |
| 4 | 3 | 1 | | 4.5 | 27.3 | 3 | 27.3 | 32.0 |
| 4 | 3 | | 2 | 5 | 25.0 | 3 | 25.0 | 42.9 |
| | 3 | 1 | 2 | 4.5 | 30.0 | 3 | 30.0 | 30.4 |
| 4 | | 1 | 2 | 4.5 | 33.3 | 3 | 33.3 | 28.6 |
| 4 | 3 | 1 | 2 | 5.5 | 28.6 | 4 | 28.6 | 24.2 |

### Example 29

### Super Solvent Effect for Ibuprofen

As shown in Table 28, the solvent systems of the present invention demonstrate remarkable super-solvent effects with ibuprofen.

TABLE 28
Super-solvent effects for Ibuprofen.

| | | | | | | | | Super- |
|---|---|---|---|---|---|---|---|---|
| PG | PEG | | | Observed | | Predicted | | solvent |
| Grams | 400 | EtOH | IPA | Ibuprofen | | Ibuprofen | | Effect |
| (gr) | gr | gr | gr | gr | % W/W | gr | % W/W | % |
| 4 | | | | 1 | 20.0 | | | |
| | 3 | | | 1 | 25.0 | | | |
| | | 1 | | 1 | 50.0 | | | |
| | | | 1.1 | 1 | 47.6 | | | |
| 4 | 3 | | | 3.5 | 33.3 | 2 | 22.2 | 50.0 |
| 4 | | 1 | | 3 | 37.5 | 2 | 28.6 | 31.3 |
| 4 | | | 1.1 | 3 | 37.0 | 2 | 28.2 | 31.5 |
| | 3 | 1 | | 2.5 | 38.5 | 2 | 33.3 | 15.4 |
| | 3 | | 1.1 | 2.5 | 37.9 | 2 | 32.8 | 15.5 |
| | | 1 | 1.1 | 2 | 48.8 | 2 | 32.8 | 48.8 |
| 4 | 3 | 1 | | 4.5 | 36.0 | 3 | 27.3 | 32.0 |
| 4 | 3 | | 1.1 | 5 | 38.2 | 3 | 27.0 | 41.2 |
| | 3 | 1 | 1.1 | 4 | 44.0 | 3 | 42.3 | 4.0 |
| 4 | | 1 | 1.1 | 4.5 | 42.5 | 3 | 33.0 | 28.8 |
| 4 | 3 | 1 | 1.1 | 6.5 | 41.7 | 4 | 30.5 | 36.5 |

### Example 30

### Super Solvent Effect for Acetaminophen

As shown in Table 29, the solvent systems of the present invention demonstrate remarkable super-solvent effects with acetaminophen.

TABLE 29
Super-Solvent Effects with Acetaminophen

| | | | | Observed | | | | Super- |
|---|---|---|---|---|---|---|---|---|
| PG | PEG | | | NSAID | | Predicted | | solvent |
| Grams | 400 | EtOH | IPA | Acetaminophen | | Acetaminophen | | Effect |
| (gr) | gr | gr | gr | gr | % W/W | gr | % W/W | % |
| 9 | | | | 1 | 10.0 | | | |
| | 9 | | | 1 | 10.0 | | | |
| | | 5 | | 1 | 16.7 | | | |
| | | | 11 | 1 | 8.3 | | | |
| 9 | 9 | | | 2.5 | 12.2 | 2 | 10.0 | 22.0 |
| 9 | | 5 | | 2.5 | 15.2 | 2 | 12.5 | 21.2 |
| 9 | | | 11 | 2.5 | 11.1 | 2 | 9.1 | 22.2 |
| | 9 | 5 | | 3.5 | 20.0 | 2 | 12.5 | 60.0 |
| | 9 | | 11 | 3.5 | 14.9 | 2 | 9.1 | 63.8 |
| | | 5 | 11 | 2.5 | 13.5 | 2 | 11.1 | 21.6 |
| 9 | 9 | 5 | | 4.5 | 16.4 | 3 | 11.5 | 41.8 |
| 9 | | 5 | 11 | 3.5 | 12.3 | 3 | 10.7 | 14.6 |
| 9 | 9 | | 11 | 4.5 | 13.4 | 3 | 9.4 | 43.3 |
| | 9 | 5 | 11 | 5 | 16.7 | 3 | 10.7 | 55.6 |
| 9 | 9 | 5 | 11 | 6.5 | 16.0 | 4 | 10.5 | 52.5 |

### Example 31

### Super-Solvent Effect with Diclofenac

As shown in Table 30, the solvent systems of the present invention demonstrate remarkable super-solvent effects with diclofenac.

Table 30: Super-Solvent Effects with Diclofenac.

### Example 32

### Super Solvent Water Effect—Flurbiprofen

This example illustrates a technical feature of compositions of the present invention; namely that the solvent systems (comprised of at least two solvent alcohols) are able to solubilize a class of NSAIDs (i.e. Drugs of the present invention) in compositions containing water at unexpectedly high levels. This super-solvent water effect is illustrated below in compositions containing a solvent system comprised of combinations of 2 or more solvent alcohols (PEG, PG, ethanol, and isopropanol) with Flurbiprofen.

Methods. First, the indicated amounts of the NSAID and solvent alcohols are

| | | | | | | | Super- |
|---|---|---|---|---|---|---|---|
| | PEG | | Observed | | Predicted | | solvent |
| PG | 400 | EtOH | Diclofenac | | Diclofenac | | Effect |
| gr | gr | gr | gr | % W/W | gr | % W/W | % |
| 6 | | | 1 | 14.3 | | | |
| | 11 | | 1 | 8.3 | | | |
| | | 6 | 1 | 14.3 | | | |
| 6 | 11 | | 3.5 | 17.1 | 2 | 10.5 | 62.2 |
| 6 | | 6 | 4.5 | 27.3 | 2 | 14.3 | 90.9 |
| | 11 | 6 | 4 | 19.0 | 2 | 10.5 | 81.0 |
| 6 | 11 | 6 | 5 | 17.9 | 3 | 11.5 | 54.8 |

combined and agitated and warmed to 35 C until dissolved to achieve a single phase composition. These amounts are chosen based upon the solubility of one gram of NSAID and the amount of the single solvent alcohol for saturation. Next, water was added in step-wise manner to determine the maximum amount that can be added before any visible amount of the NSAID precipitated (i.e., the volume of water that can be added and yet remain single phasic). The super-solvent water effect was determined using this protocol, where per cent=actual grams of water added divided by the predicted grams (based upon the first step) expressed as per cent increase (e.g. if twice the predicted amount could be solubilized, the super-solvent effect would be % 100). The results are shown in Table 31

TABLE 31
The Super-Solvent Water Effect - Flurbiprofen

| | | | | | | Water | | Water | | Super- |
|---|---|---|---|---|---|---|---|---|---|---|
| | | | | | | Observed | | Predicted | | Solvent |
| Flurbiprofen | PG | PEG | EtOH | IPA | Flurbiprofen | | % | | % | Effect |
| gm | gm | gm | gm | gm | % W/W | gm | W/W | gm | W/W | |
| 1 | 6 | | | | 12.5 | 1 | | | | |
| 1 | | 2 | | | 25.0 | 1 | | | | |
| 1 | | | 2 | | 20.0 | 2 | | | | |
| 1 | | | | 3 | 14.3 | 3 | | | | |
| 2 | 6 | 2 | | | 14.3 | 4 | 40.0 | 2.0 | 16.7 | 140.0 |
| 2 | 6 | | 2 | | 12.5 | 6 | 60.0 | 3.0 | 23.1 | 160.0 |
| 2 | 6 | | | 3 | 11.8 | 6 | 54.5 | 4.0 | 26.7 | 104.5 |
| 2 | | 2 | 2 | | 25.0 | 2 | 33.3 | 3.0 | 33.3 | 0.0 |
| 2 | | 2 | | 3 | 15.4 | 6 | 85.7 | 4.0 | 36.4 | 135.7 |
| 2 | | | 2 | 3 | 18.2 | 4 | 57.1 | 5.0 | 41.7 | 37.1 |
| 3 | 6 | 2 | 2 | | 17.6 | 4 | 30.8 | 4.0 | 23.5 | 30.8 |
| 3 | 6 | 2 | | 3 | 13.6 | 8 | 57.1 | 5.0 | 26.3 | 117.1 |
| 3 | 6 | | 2 | 3 | 13.6 | 8 | 57.1 | 6.0 | 30.0 | 90.5 |
| 3 | | 2 | 2 | 3 | 18.8 | 6 | 60.0 | 6.0 | 37.5 | 60.0 |
| 4 | 6 | 2 | 2 | 3 | 15.4 | 9 | 52.9 | 7.0 | 29.2 | 81.5 |

The results above illustrate an optional technical feature of compositions of the present invention, namely that when a solvent system is combined with an NSAID, high concentrations of drug can be achieved and that such composition can tolerate water in unrepentantly high amounts.

### Example 33

### Super Solvent Water Effect—Ibuprofen

Using the protocol accompanying the data of Table 31, the super-solvent water effect for ibuprofen was examined. As shown in Table 32, an optional technical feature of compositions of the present invention are solvent alcohols with a capacity to dissolve high levels of ibuprofen and water and remain single phasic.

TABLE 32
Super-Solvent Water Effect - Ibuprofen

| | | | | | | | | | | Super- |
|---|---|---|---|---|---|---|---|---|---|---|
| | | | | | | Water | | Water | | solvent |
| Ibuprofen | PG | PEG | EtOH | IPA | Ibuprofen | Observed | | Predicted | | Effect |
| gm | gm | gm | gm | gm | % W/W | gm | % W/W | gm | % W/W | on water |
| 1 | 4 | | | | 11.4 | 1.9 | 21.6 | | | |
| 1 | | 3 | | | 13.3 | 1.8 | 23.3 | | | |
| 1 | | | 1 | | 37.7 | 0.3 | 12.3 | | | |
| 1 | | | | 1.1 | 36.4 | 0.3 | 11.8 | | | |
| 2 | 4 | 3 | | | 11.7 | 4.1 | 45.0 | 3.7 | 28.9 | 56.0 |
| 2 | 4 | | 1 | | 15.9 | 2.8 | 40.0 | 2.2 | 24.1 | 65.8 |
| 2 | 4 | | | 1.1 | 15.7 | 2.8 | 39.4 | 2.2 | 23.9 | 65.3 |
| 2 | | 3 | 1 | | 19.0 | 2.3 | 37.5 | 2.1 | 25.7 | 45.9 |
| 2 | | 3 | | 1.1 | 18.3 | 2.4 | 39.3 | 2.1 | 25.4 | 55.0 |
| 2 | | | 1 | 1.1 | 37.4 | 0.6 | 15.2 | 0.7 | 13.7 | 11.4 |
| 3 | 4 | 3 | 1 | | 14.9 | 4.6 | 41.8 | 4.0 | 26.5 | 57.5 |
| 3 | 4 | 3 | | 1.1 | 14.8 | 4.6 | 41.4 | 4.0 | 26.4 | 57.2 |
| 3 | | 3 | 1 | 1.1 | 22.6 | 2.6 | 32.1 | 2.4 | 22.9 | 40.4 |
| 3 | 4 | | 1 | 1.1 | 19.2 | 3.3 | 35.7 | 2.6 | 21.9 | 63.2 |
| 4 | 4 | 3 | 1 | 1.1 | 17.5 | 4.9 | 37.0 | 4.3 | 24.7 | 49.8 |

### Example 34

### Super Solvent Water Effect—Ketoprofen

Using the protocol accompanying the data of Table 31, the super-solvent water effect for ketoprofen was examined. As shown in Table 33, the super-solvent water effect for the indicated solvent systems was less than remarkable as compared to that observed for Ibuprofen and flurbiprofen.

TABLE 33
Super-Solvent water effect - Ketoprofen

| | | | | | | | | | | Super- |
|---|---|---|---|---|---|---|---|---|---|---|
| | | | | | | | | | | solvent |
| | | | | | | Water | | Water | | Effect |
| Ketoprofen | PG | PEG | EtOH | IPA | Ketoprofen | Observed | | Predicted | | on water |
| gm | gm | gm | gm | gm | % W/W | gm | % W/W | gm | % W/W | % |
| 1 | 4 | | | | 20.0 | 3 | 37.5 | | | |
| 1 | | 3 | | | 25.0 | 3 | 42.9 | | | |
| 1 | | | 1 | | 50.0 | 0.5 | 20.0 | | | |
| 1 | | | | 2 | 33.3 | 1.5 | 33.3 | | | |
| 2 | 4 | 3 | | | 22.2 | 6 | 40.0 | 6.0 | 40.0 | 0.0 |
| 2 | 4 | | 1 | | 28.6 | 4 | 36.4 | 3.5 | 33.3 | 9.1 |
| 2 | 4 | | | 2 | 25.0 | 5 | 38.5 | 4.5 | 36.0 | 6.8 |
| 2 | | 3 | 1 | | 33.3 | 3 | 33.3 | 3.5 | 36.8 | −9.5 |
| 2 | | 3 | | 2 | 28.6 | 5 | 41.7 | 4.5 | 39.1 | 6.5 |
| 2 | | | 1 | 2 | 40.0 | 2 | 28.6 | 2.0 | 28.6 | 0.0 |
| 3 | 4 | 3 | 1 | | 27.3 | 6 | 35.3 | 6.5 | 37.1 | −5.0 |
| 3 | 4 | 3 | | 2 | 25.0 | 8 | 40.0 | 7.5 | 38.5 | 4.0 |
| 3 | 4 | | 1 | 2 | 30.0 | 6 | 37.5 | 5.0 | 33.3 | 12.5 |
| 3 | | 3 | 1 | 2 | 33.3 | 6 | 40.0 | 5.0 | 35.7 | 12.0 |
| 4 | 4 | 3 | 1 | 2 | 28.6 | 9 | 39.1 | 8.0 | 36.4 | 7.6 |

### Example 35

### Maximal NSAID and Water Concentrations—PG, PEG, and Naproxen

The indicated amounts of naproxen and solvent alcohols were mixed to form single phasic compositions and water was added in aliquots until the precipitation point (i.e. the point where single phasic character is lost).

TABLE 34
Maximal Naproxen and water Concentrations with PG and PEG.

| | | | Water Added |
|---|---|---|---|
| | | PEG | Until |
| | PG | 400 | Precipitation |
| Naproxen (gm) | (gm) | (gm) | Occurs (gm) |
| 0.1215 | 2 | 2 | 3.5 |
| 0.324 | 2 | 2 | 1 |
| 0.405 | 2 | 2 | 0.5 |
| 0.1755 | 1 | 3 | 3.5 |
| 0.468 | 1 | 3 | 1 |
| 0.585 | 1 | 3 | 0.5 |
| 0.27 | 1 | 4 | 4.5 |
| 0.54 | 1 | 4 | 2.5 |
| 0.675 | 1 | 4 | 1 |
| 0.2295 | 3 | 4 | 6.5 |
| 0.612 | 3 | 4 | 2.5 |
| 0.765 | 3 | 4 | 1.5 |
| 0.1755 | 4 | 3 | 5 |
| 0.468 | 4 | 3 | 2.5 |
| 0.585 | 4 | 3 | 1.5 |

FIG. 8A shows a plot of the water concentration in % (“W”) and naproxen (“D”) at the highest levels that were obtainable while retaining the single phase feature of the present invention. These per cents were inversely correlated and a linear regression analysis of data points reveals a significant correlation coefficient (r²=0.8354).

These data reveal that one skilled in the art can now formulate present compositions to achieve desired physicochemical properties. For example, compositions with high Drug concentrations and substantial amounts of water can be predictably made.

In the example below, the solvent systems comprised PG and PEG in total combined amounts of 50% to 82% and the PG to PEG ratio was from 0.33 to 1.3. Drug concentrations ranged up to about 13% and water concentrations ranged from about 10% to about 47%. The maximum Drug and water concentrations for any given composition can be described by W=−3.5(D)+49 [Formula 13, where

W=−3.5(D)+49 [Formula 13].

Accordingly, in one embodiment of the invention, compositions comprise a propylene glycol, a polyethylene glycol, water and naproxen, where the propylene glycol and the polyethylene glycol are present in a combined amount from about 50% to about 82% of composition, and wherein the ratio of propylene glycol to polyethylene glycol (PG/PEG) is from about 0.33 to about 1.33, and wherein the amount of water and Drug in the composition is expressed as

W≦−3.5(D)+49 where W and D are positive. Formula 14

Drug and water concentrations, when compositions comprise a propylene glycol, a polyethylene glycol, water and naproxen, where the propylene glycol and the polyethylene glycol are present in a combined amount from about 50% to about 82% of composition, and wherein the ratio of propylene glycol to polyethylene glycol (PG/PEG) is from about 0.33 to about 1.33, can be described by Formula 15

W=3.5(D)+B, wherein B is between zero and about 49. Formula 15

Optionally B is about any of the following ranges: 10-49 or 15-49 or 30-49, 10 to 20, 15 to 25, 20 to 30, 25 to 35, or 30 to 40. Within any of compositions described by the preceding B ranges, the Drug concentration can optionally be about the following: 5 -30 or 10-30 or 15 to 30 or 10-20. Each of the compositions described above (i.e. where B is equal to or less than 49) are single phasic.

FIG. 8B shows the data in this example plotted according to PEG to PG ratio. Accordingly, water and drug concentrations can be formulated with Naproxen according to the formulations below.

TABLE 35
Naproxen, Water, and Solvent Systems

| | | B | B |
|---|---|---|---|
| PG/PEG | | minimum | maximum |
| 2/2 | y = −4.3305x + b | 0 | 54.158 |
| 1/3 | y = −3.4276x + b | 0 | 52.794 |
| 1/4 | y = −4.5403x + b | 0 | 47.637 |
| 3/4 | y = −4.921x + b | 0 | 52.964 |
| 4/3 | y = −3.6923x + b | 0 | 56.913 |

### Example 36

### Maximal NSAID and Water Concentrations—PG, PEG, and Ketoprofen

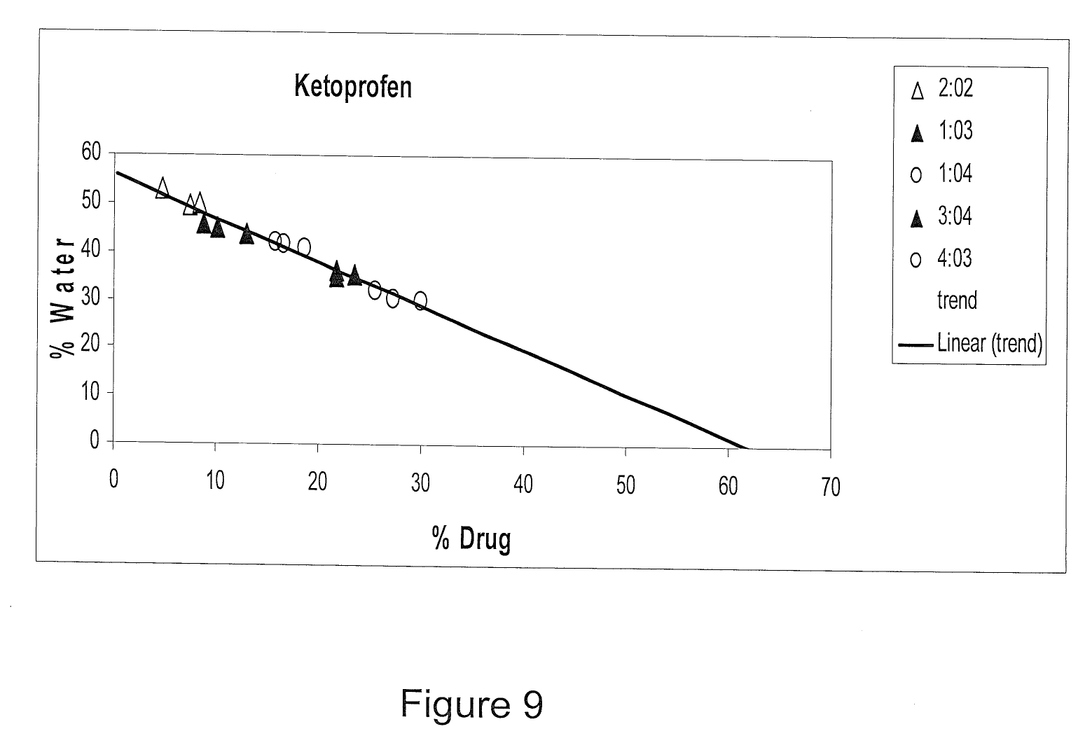

In a manner similar to that which generated Table 34, maximal water and Dug concentrations in solvent systems of the present invention were determined and plotted (FIG. 9) as a line described by:

y=−0.905x+55.9. Formula 16

### Example 37

### Maximal NSAID and Water Concentrations—PG, PEG, and Ibuprofen

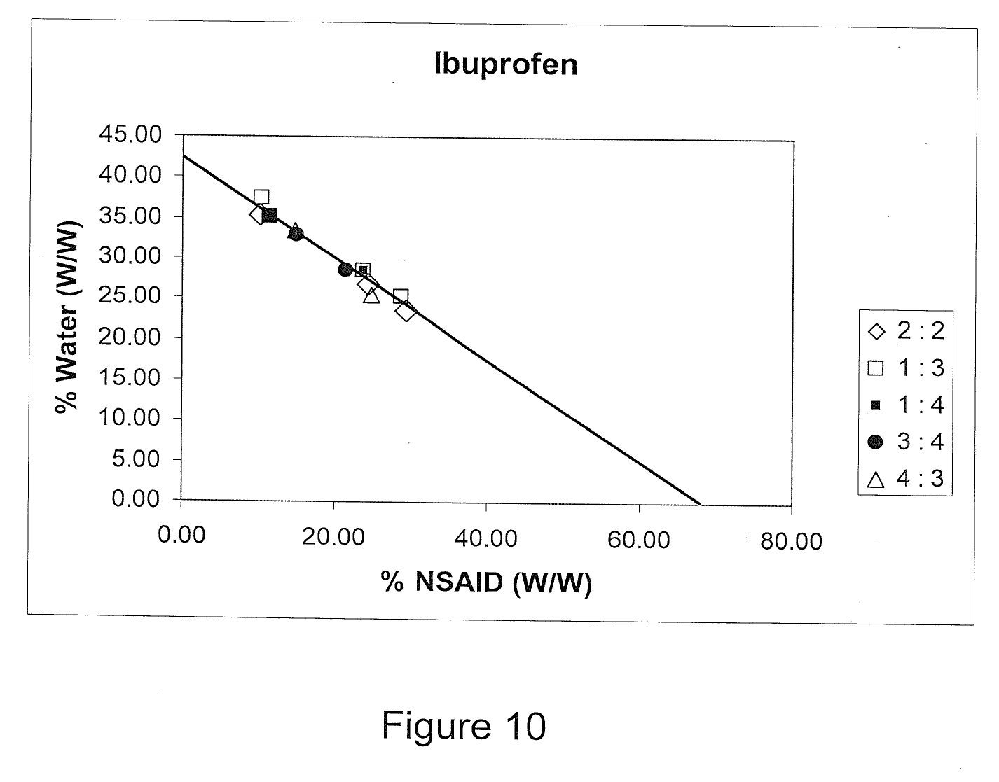

In a manner similar to that which generated Table 34, maximal water and Dug concentrations in solvent systems of the present invention were determined for ibuprofen and plotted in FIG. 10 as a line described by:

W=−0.62D+42.5. [Formula 17]

Useful compositions can be described by, for example:

W=−0.62D+b where b is 0 to 42.5 [Formula 18] or

W<−0.62D+42.5 [Formula 19]

### Example 38

### Maximal NSAID and Water Concentrations—PG, PEG, and Acetaminophen

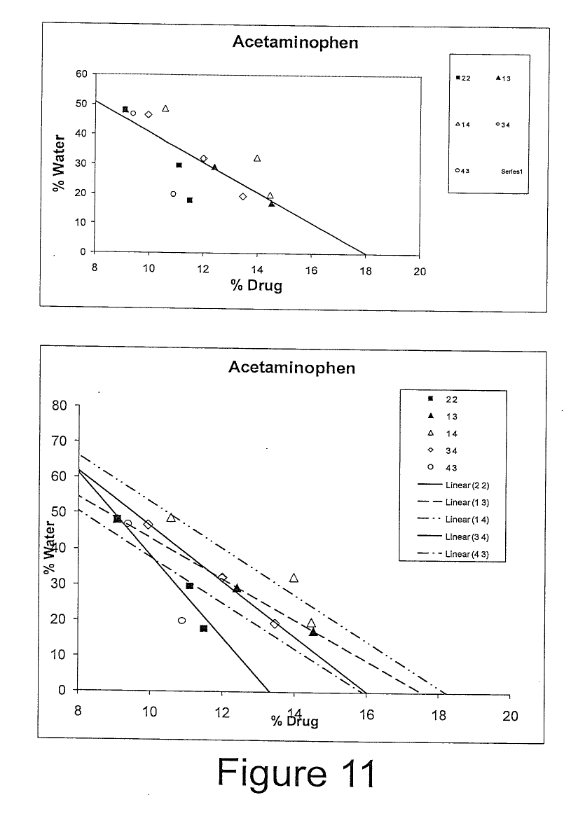

In a manner similar to that which generated Table 34, maximal water and Dug concentrations in solvent systems of the present invention were determined for acetaminophen and plotted in FIG. 11A as a line described by:

y=−5.0683x+91.415 Formula 20

FIG. 11B shows the data in this example plotted according to PEG to PG ratio. Accordingly, water and drug concentrations can be formulated with Naproxen according to the formulations below.

TABLE 36
Maximum Water and Acetaminophen Concentrations

| | y = water | | |
|---|---|---|---|
| | concentration (%) | | |
| | W/W) | | |
| | x = Drug | | |
| | concentration (% | B | B |
| PG/PEG | W/W) | minimum | maximum |
| 2/2 | y = −11.52x + b | 0 | 154 |
| 1/3 | y = −5.7055x + b | 0 | 100 |
| 1/4 | y = −6.4787x + b | 0 | 118 |
| 3/4 | y = −7.7243x + b | 0 | 124 |
| 4/3 | y = −6.3927x + b | 0 | 102 |

### Example 39

### Determining Pharmacokinetics, Pharmacodynamics, Therapeutic Efficacy, and System Absorption

Compositions as set forth in Example 12 through Example 26 are tested in comparison to the composition of Example 8 (high alkanol comparator composition). The Drug (or NSAID) in each compositions is ibuprofen.

Pharmacokinetics are determined by microdialysis (set forth in Example 7), by minipig tests (set forth in Example 2), and by in vitro percutaneous penetration (as set forth in Example 6).

Therapeutic efficacy is determined by mouse edema ear assay (as set forth in Example 1) and in PFB (as set forth in Example 4). Compositions have one or more superior properties taught herein.

Systemic absorption is determined and compared to systemic levels following oral administration as set forth in Example 3. Systemic levels following topical administration are substantially below levels commonly associated with gastrointestinal side effects.

### Example 40

### In Vitro Percutaneous Penetration

The compositions of Example 39 are examined for penetration of Drug into the skin as described in Example 6 utilizing human abdominal skin in a modified Franz (Bronough) flow-through diffusion cell. Dermatomed human abdominal skin is treated with formulations containing ³H-Drug at approximately 1 μCi/dose using the procedures described in the FDA/AAPS workshop report on in vitro percutaneous penetration studies. After a 24-hour exposure period, the amount of radioactivity in skin wipes, tape-strips, and epidermis, dermis, and receptor fluid samples is determined using liquid scintillation counting. Useful ranges are shown in Table 37.

TABLE 37
In Vitro Percutaneous Absorption of Drug as Detected by the Franz Assay
Over a 24-Hour Period

| | Amount | Amount | | | |
|---|---|---|---|---|---|
| | found in | found in | | Total Dose | X |
| | Receptor | Receptor | Amount | Recovered | intercept |
| Formulation | Fluid^{a} (%) | Fluid (μg) | on tape | (%) | (Hours)¹ |
| | 4-10% | 5-40 | 5-10 μg | 85-100% | −10-10 |

¹X-axis intercept calculated at pseudo steady-state flux (12 through 24 hours)

Receptor fluid analysis approximates the amount of compound that would reach the systemic circulation in vivo. The penetration of Drug through the skin after 24 hours ranges from approximately 12 to 43 μg of ibuprofen for the formulations examined in this study. Epidermal levels after tape-stripping ranges from 7 to 100 μg while dermal deposition rangez from 1 to 10 μg of Drug over this same time period.

When the time-intercept for absorption is calculated at pseudo steady-state flux (12 through 24 hours), present compositions can sometimes demonstrate a negative lag time. A negative lag time indicates a rapid and immediate uptake of Drug from the formulation into hair follicles and other skin appendages. For example, the negative lag time may result from the solvent in the formulation delivering Drug rapidly into the sebum rich areas. The absorption characteristics of present compositions should lead to rapid absorption and distribution of Drug into the target tissues yet have limited systemic bioavailability of ibuprofen.

Based up these studies, 0.5 gram of a present composition containing 75 mg of Drug BID (i.e. 150 mg/day of Drug) can have a percutaneous absorption of below the exposure resulting from the recommended dose of over-the-counter Drug in the United States (e.g. below 20 mg/day).

### Example 41

### Minipig Studies

One gram of the compositions of Example 39 are applied daily to a 10 cm×20 cm area on the back of minipigs in a thin layer for 13 weeks

After 13 weeks of treatment there is no treatment-related changes in any of the clinical parameters evaluated. One or more animals in all groups including the control group has occasional erythema at the application site, but there is no relationship to the test article and no statistically significant difference between the control group and any test groups. No skin edema is seen in any animal during the first 6 weeks of the study. Animals sacrificed at the 6-week interim time point have no treatment-related changes in organ weights or in gross or microscopic pathology evaluations.

Drug is identified at low levels in plasma at each time point in all animals. Tₘₐₓ is reached at about 1 to 5 hours. Cₘₐₓ varies between approximately 20 and 200 ng/mL. Steady state appears to be reached on Day 1.

No compositions are significant dermal irritants as are measured by the modified Draize test in rabbits and none sensitize skin (as defined by a murine local lymph node stimulation assay).

### Example 42

### Efficacy in PFB

Compositions of Example 39 are evaluated in the PFB protocol set forth in Example 4. Results indicate that low alkanol compositions containing an NSAID of the phenylacetic acid type are effective to reduce severity of PFB in mild, moderate, and severe PFB.

Moreover, compositions are also effective in treatment of PFB with an “every-other-day” application regimen. Test subjects with acne or dermatitis (e.g. contact dermatitis) also report therapeutic efficacy against there indications.

Certain subjects report sensitivity to alkanols yet do not show adverse reaction to present compositions

Test results are comparable to those of subjects treated with the composition of Example 8 (high alkanol comparator composition). However, subjects report less of a drying effect and less stinging of razor cuts. This later observation is especially important when skin is especially sensitive (e.g. legs, pubic region, etc.).

Certain subjects, in the normal course of their disease, routinely experience more severe inflammation around razor bumps, nodulocystic lesions, erythema, and hyperpigmentation. Such subjects report improvement of such pathologies.

### Example 43

### Dermal Irritation Testing

Compositions of Example 39 are tested for acute dermal irritation in New Zealand White rabbits. One male and 2 female rabbits are treated for each composition. The compositions are applied topically to a 10 cm×10 cm site on the dorsal trunk on each rabbit. The application sites are wrapped for 4 hours after which time the wrappings are removed. Irritation scores are performed at 60 minutes after removal of the wrap and again at 24, 48 and 72 hours. No significant erythema or edema is seen at any observation period. The compositions are considered negative for dermal irritation in this assay.

### Example 44

### Lymph Node Stimulations Test

Compositions of Example 39 are subjected to a local lymph node stimulation assay in CBA/J female mice to determine if present compositions produce a hypersensitivity response as measured by the proliferation of lymphocytes in the draining lymph nodes. Ten groups of 5 mice each are used. Five groups are treated on the dorsal surface of both ears once per day for 3 days the compositions or with a positive control (35% hexylcinnamaldehyde (HCA)). On Day 6, the mice are injected intravenously (IV) with 20 μCi of ³H-thymidine in sterile saline. Five hours later the mice are euthanized and the draining auricular lymph nodes are removed. The lymph node cells are precipitated with 5% trichloroacetic acid and the pellets are counted by scintillation counting to determine the incorporation of ³H-thymidine.

A three-fold or greater increase in proliferative activity relative to the concurrent vehicle-treated control is considered a positive response. The positive control, 35% HCA in ISW-AP-01 placebo, results in a stimulation index of 4.6. The positive control, 35% HCA in ethanol, results in a stimulation index of 4.4. Since both of these stimulation indices are greater than 3, the positive controls indeed produced a positive response.

Animals treated with present compositions of Example 39 have stimulation indices of less than 1 or 2 and are considered to not have skin-sensitizing activity.

### Example 45

### Compositions with NSAID ProDrug and NSAID Drug

Compositions are optionally formulated according to Table 38. Each NSAID prodrug (expressed as “parent compound plus pro-moiety in Table 38) or NSAID is formulated four different ways: as a high total Drug concentration (e.g. 15-30%) (“A”); as a low alkanol concentration (e.g. <about 15%-about 30%) (“B”); as a high water concentration (e.g. about 20 to about 50%) (“C”); and a high drug, low alkanol, and high water concentration combined with a second Drug (e.g. Drug plus antibiotic or antifungal or antihistamine or antipsoriatic or second NSAID) (“D”). The compositions are formulated according to the teaching in the present invention and by consideration of the physicochemical properties of each drug. Each composition is prepared at three pH's: 4.0, 5.0, 6.0.

Drug absorption, distribution, metabolism and elimination are determined in ex vivo and in vivo animal models.

Efficacy is measured in the contact dermatitis model in the hairless guinea pig (for example, J Dermatol. 1992 Mar;19(3):140-5.), psoriasis in the mouse model overexpressing amphiregulin, Atopic Dermatitis in the Epidermal Interleukin-4 transgenic mouse model, (Journal of Investigative Dermatology Volume 117 Issue 4 Page 977—October 2001), and other models.

All data are analyzed using nonparametric analysis of variance. Models are generated to aid in the selection and optimization of NSAID (and/or NSAID prodrug) and formulation for various inflammatory skin disorders.

TABLE 38
NSAIDs and NSAID Pro-drugs of the present Compositions

| NSAID | | |
|---|---|---|
| parent compound | Pro-moiety (e.g. ester/ether) | Formulation |
| dicoflenac | — | A, B, C, D |
| flubiprofen | — | A, B, C, D |
| ibuprofen | — | A, B, C, D |
| naproxen | | |
| acetaminophen | | |
| ibuprofen | | |
| Ketoprofen | — | A, B, C, D |
| bufexamac | methyl (i.e. bufexamac methyl ester) | A, B, C, D |
| dicoflenac | ethyl | A, B, C, D |
| etofenamate | isopropyl | A, B, C, D |
| Felbinac | n-butyl | A, B, C, D |
| entiazac | palmityl | A, B, C, D |
| fepradinol | 4-(nitrooxy)butyl | A, B, C, D |
| flufenamic | Dimethylformamidyl | A, B, C, D |
| lunoxaprofen | alcoholic xyethyl | A, B, C, D |
| flubiprofen | isopropyloxy | A, B, C, D |
| ibuprofen | Ethyl or isopropyl | A, B, C, D |
| indomethacin | isopropyl | A, B, C, D |
| sonixin | isopropyloxy | A, B, C, D |
| Ketoprofen | lauryl | A, B, C, D |
| ketorolac | N-ethyloxy N-propyl N-ethyl amino | A, B, C, D |
| Niflumic | p-alcoholic xyphenylurea | A, B, C, D |
| Oxyphenbutazone | polyethylene glycyl | A, B, C, D |
| piketoprofen | polyethylenyl | A, B, C, D |
| piroxicam | propylene glycoxymercaptoethyl | A, B, C, D |
| pranoprofen | triethylamino | A, B, C, D |
| suxibuzone | N-ethyloxy, N-propyl, N-ethyl, | A, B, C, D |
| | aminoethyl | |
| ufenamate | ethyl | A, B, C, D |

1. A composition comprising an NSAID, a solvent system, and from zero to about 60% alkanol, wherein
(i) the solvent system comprises at least two solvent alcohols;
(ii) one of the at least two solvent alcohols is a polyethylene glycol, a propylene glycol, glycerin, polyether polyol, butylene glycol; or a glycerol derivative;
(iii) the solvent system has at least about a 20% super solvent effect;
(iv) the solvent system is present in an amount sufficient to solubilize the NSAID;
(v) the NSAID is in an amount of about 12% to about 30%; and
(vi) the composition is single phasic.

2. The composition of claim 1 wherein the other one of at least two solvent alcohols is selected from the group consisting of polyethylene glycols, propylene glycols, glycerin, polyether polyols, butylene glycols; glycerol derivatives, ethanol, and isopropanol.

3. The composition of claim 1 wherein the NSAID is in an amount of from about 0.25 to about 2 times the value determined by Formula 1.

4. The composition of claim 1 further comprising at least about 20% water.

5. The composition of claim 1 wherein the at least two solvent alcohols are polyethylene glycol and propylene glycol in a combined amount of about 40% to about 60% and wherein the composition further comprises water wherein water and the NSAID are in amounts according to W=−0.62D+b where b is 0 to 42.5 [Formula 18] or according to W≦−0.62D+42.5 [Formula 19].

6. The composition of claim 5, wherein the NSAID has a pKₐ from about 3.0 to about 6.5 and a log₁₀ P value of about 2 to about 5.5.

7. The composition of claim 1 wherein, when dermally applied daily, is effective in treating PFB.

8. The composition of claim 1 wherein the composition further comprises an NSAID prodrug.

9. The composition of claim 1 further comprising a botanical agent.

10. The composition of claim 1 wherein one of at least two solvent alcohols is an alkanol and upon storage at room temperature, there is less than 1% NSAID alkanol ester formed.

11. The composition of claim 1 wherein the alkanol is about 0 to one of about 40%, about 30%, about 20%, or about 10%.

12. The composition of claim 1 having a viscosity in a range selected from the group of ranges consisting of about 2000 cps to about 200,000 cps, about 50,000 cps to about 200,000 cps, about 50,000 cps to about 100,000 cps, about 2,000 to about 50,000, about 2,000 cps to about 25,000 cps, about 2,000 cps to about 10,000 cps, and about 2,000 cps to about 5,000 cps.

13. The composition of claim 1 where the Drug is an NSAID prodrug and where the composition further comprises a thickening agent, wherein the NSAID is of the phenylacetic acid-type and the pro-moiety is an amidyl, a thio, or an alkyl in ester linkage to the NSAID.

14. A method of treating a subject with dermatophytoses, seborrheic dermatitis, or pityriasis capitis comprising applying the composition of claim 1 wherein the composition further comprises an antifungal agent.

15. A method of treating a subject with a furuncle, Impetigo, a wound, a skin infection, rosacea, or Pseudomonas folliculitis comprising dermally applying the composition of claim 1 wherein the composition further comprises an antibacterial agent

16. A method of treating a subject in need comprising dermally applying the composition of claim 1 wherein the composition further comprises a corticosteroid and wherein the NSAID is in a corticosteroid-sparing amount.

17. A method of treating a subject with PFB, dermatitis, sunburn, actinic keratoses, rosacea, vitiligo, a wound, or a skin infection, comprising dermally applying the composition of claim 1 wherein the composition further comprises one or more of a UVA blocker, a UVB blocker, and an antioxidant.

18, A method of treating a subject in need comprising dermally applying the composition of claim 1 wherein the composition further comprises an antihistamine.

19. A method of treating a subject in need comprising dermally applying the composition of claim 1 wherein the composition further comprises a botanical agent.
